(19) United States
(12) Patent Application Publication
(10) Pub. No.: US 2026/0130980 A1
(43) Pub. Date: May 14, 2026

(54) SYNTHETIC MULTIVALENT TUBERCULOSIS VACCINE

(71) Applicant: The Wistar Institute of Anatomy and Biology, Philadelphia, PA (US)

(72) Inventors: David Weiner, Merion, PA (US); Elizabeth Parzych, Philadelphia, PA (US)

(21) Appl. No.: 19/118,630

(22) PCT Filed: Oct. 6, 2023
(86) PCT No.: PCT/US2023/076240

§ 371 Date: Apr. 4, 2025

Related U.S. Application Data
(60) Provisional application No. 63/378,533, filed on Oct. 6, 2022.

(51) Int. Cl. A61K 39/04; A61K 39/00; A61P 37/04; C07K 14/35

(52) U.S. Cl. CPC A61K 39/04; A61P 37/04; C07K 14/35; A61K 2039/53; A61K 2039/70

(57) ABSTRACT

Compositions comprising a nucleic acid molecule that encodes TB proteins are disclosed. Methods of inducing an immune response against TB in an individual are disclosed. Method of treating an individual who has been diagnosed with TB are disclosed. Method of preventing TB infection in an individual are disclosed.

### CROSS REFERENCE TO RELATED APPLICATIONS

This application claims priority to U.S. Provisional Application No. 63/378,533, filed Oct. 6, 2022 which is hereby incorporated by reference herein in its entirety.

### STATEMENT REGARDING FEDERALLY SPONSORED RESEARCH OR DEVELOPMENT

This invention was made with government support under AI135723 awarded by the National Institutes of Health. The government has certain rights in the invention.

### REFERENCE TO A “SEQUENCE LISTING,” SUBMITTED AS AN XML FILE

The Sequence Listing written in the xml file titled: “206193-0095-00WO_SequenceListing”; created on Oct. 6, 2023, and 102,905 bytes in size, is hereby incorporated by reference.

### FIELD OF THE INVENTION

The present invention relates to multivalent constructs encoding tuberculosis (TB) immunogens encoding immunogenic TB antigens. Each construct encodes multiple immunogenic TB antigens and has coding sequences designed for high levels of expression. Prophylactic and therapeutic vaccines, and methods of making and using the same to induce immune responses, preventing TB infection and treat individuals infected with TB virus are provided.

### BACKGROUND OF THE INVENTION

Tuberculosis (TB) is a major infectious disease with significant morbidity and mortality worldwide. The only currently licensed vaccine against TB is the Bacillus Calmette-Guerin (BCG) vaccine. Unfortunately, this vaccine confers poor protection against adult pulmonary TB and has been associated with adverse events. Therefore, the development of a novel, effective vaccine that induces long-term protection against TB is urgently needed. However, due to a variety of factors only a few antigens which have been determined to induce T cell immunity against TB have been studied so far. These include Ag85A, Ag85B, ESAT6, TB10.4, and Mtb39a. One issue is that there are many TB antigens from which to choose and current technologies for delivering TB antigens are limited and expensive.

There remains a need for economical and effective TB vaccines and methods that can induce immune responses against immunogenic TB antigens, protect against TB infection and provide effective treatment to individual who are infected with TB. There is also a need for a cost-effective delivery system to enable mass prophylactic vaccination against TB.

### BRIEF DESCRIPTION OF THE DRAWINGS

FIG. 1A and FIG. 1B depict the design and in vitro expression of vaccine plasmids. FIG. 1A depicts multivalent construct designs. Synthetic inserts were designed and synthesized containing an N-terminal IgE leader sequence, followed by the codon optimized sequences encoding the indicated M. tb antigens (Rv strain) separated by furin cleavage sites. These inserts were cloned into a pVax vector between the CMV-promotor (proCMV) and poly-adenosine sites (BGH pA). FIG. 1B depicts plasmid expression by IFA. Cell lines were transfected with the indicated plasmids. Expression of vaccine antigens was detected by IFA using mouse antisera raised against the indicated constrict (left) compared to control sera (right). Mtb-specific antibodies were detected using FITC-labeled, anti-mouse (IgG) and nuclei were visualized by DAPI.

FIG. 2A and FIG. 2B depict data demonstrating that multivalent vaccines induce broad and potent antigen-specific T cell responses. FIG. 2A depicts the experimental layout. FIG. 2B depicts bar graphs quantifying IFNg production via ELISpot analysis in harvested splenocytes that were stimulated with antigen-specific peptide pools.

FIG. 3A and FIG. 3B depict data demonstrating that multivalent vaccines induce both CD4+ and CD8+, polyfunctional cellular responses following a BCG prime. FIG. 3A depicts the experimental layout. FIG. 3B depicts bar graphs quantifying the total percentage of CD8+ and CD4+ cells expressing IFNg, TNFa, and/or IL-2 (top) or bifunctional for IFNg+/TNFa+ (bottom).

FIG. 4 depicts data demonstrating that vaccines prolong bacterial control following experimental M. tuberculosis challenge in BCG-primed animals. Mice were immunized as previously depicted and challenged with M. tuberculosis. Half of each group (n=8) was sacrificed 4 wks (top) or 12 wks (bottom) post challenge and bacterial burden was quantified in the lung (left) and spleen (right).

FIG. 5A and FIG. 5B depict the design and immunogenicity of ESX-based vaccines. FIG. 5A depicts a schematic of the generation of two additional vaccine plasmids encoding select antigens derived from the parental pEsx construct. FIG. 5B depicts an experiment where mice were immunized twice with the indicated vaccines and sacrificed two weeks later. Harvested splenocytes were stimulated with peptide pools representing the indicated antigens and assessed for IFNg production by ELISpot.

FIG. 6A and FIG. 6B depict data demonstrating that Mtb antigen EsxR is required and sufficient to confer pESX-mediated protection. FIG. 6A depicts the experimental design. FIG. 6B depicts a bar graph quantifying the bacterial burden in the lung (left) and spleen (right). Mice were immunized as previously depicted and challenged with M. tuberculosis. A subset of each group was sacrificed 4 weeks (top) or 12 wks (bottom) post challenge.

### SUMMARY OF THE INVENTION

In some embodiments, the invention provides a multivalent vaccine approach to induce broad immune responses. In some embodiments, the invention relates to nucleic acid molecules comprising a nucleotide sequence encoding one or more tuberculosis (TB) antigen. In some embodiments, the nucleic acid molecule encodes one or more of: Rv3017c (EsxQ), Rv3020c (EsxS), Rv3019c (EsxR), Rv3891c (EsxD), Rv2346c (EsxO), Rv3445c (EsxU), Rv3619c (EsxV), Rv3875 (EsxA), Rv3874 (EsxB), Rv3136c (PPE51), Rv3615c (EspC), Rv1009c (Rpf B), Rv2034c, Rv2628c, Rv2719c, Rv0010c, Rv1872c, Rv0012, Rv0090c, Rv0095, Rv1886c (Ag85B), Rv1733c, or Rv2626c.

In some embodiments, the nucleic acid molecule comprises nucleotide sequences encoding a combination of EsxQ, EsxS, EsxR, EsxD, EsxO, EsxU, EsxV, EsxA, and EsxB tuberculosis antigens. In some embodiments, the nucleic acid molecule comprises nucleotide sequences encoding a combination of PPE51, EspC, Rpf B, Rv2034c, and Rv2628c tuberculosis antigens. In some embodiments, the nucleic acid molecule comprises nucleotide sequences encoding a combination of Rv2719c, Rv0010c, Rv1872c, Rv0012, Rv0090c, and Rv0095 tuberculosis antigens. In some embodiments, the nucleic acid molecule comprises nucleotide sequences encoding a combination of Ag85B, EsxA, Rv1733c, Rv2626c, and Rpf B tuberculosis antigens. In some embodiments, the nucleic acid molecule comprises nucleotide sequences encoding a combination of EsxQ, EsxD, EsxO, EsxU, EsxV, EsxA, and EsxB tuberculosis antigens. In some embodiments, the nucleic acid molecule comprises a nucleotide sequence encoding EsxR tuberculosis antigen.

In some embodiments, the nucleic acid molecule encodes one or more amino acid sequence of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:44, or SEQ ID NO:46. In some embodiments, the nucleic acid molecule encodes a combination of SEQ ID NO:1, SEQ ID NO:3, SEQ ID NO:5, SEQ ID NO:7, SEQ ID NO:9, SEQ ID NO: 11, SEQ ID NO:13, SEQ ID NO:15, and SEQ ID NO:17. In some embodiments, the nucleic acid molecule encodes a combination of SEQ ID NO:19, SEQ ID NO:21, SEQ ID NO:23, SEQ ID NO:25, and SEQ ID NO:27. In some embodiments, the nucleic acid molecule encodes a combination of SEQ ID NO:29, SEQ ID NO:31, SEQ ID NO:33, SEQ ID NO:35, SEQ ID NO:37, and SEQ ID NO:39. In some embodiments, the nucleic acid molecule encodes a combination of SEQ ID NO:41, SEQ ID NO:15, SEQ ID NO:44, SEQ ID NO:46, and SEQ ID NO:23. In some embodiments, the nucleic acid molecule encodes a combination of SEQ ID NO:1, SEQ ID NO:7, SEQ ID NO:9, SEQ ID NO:11, SEQ ID NO:13, SEQ ID NO:15, and SEQ ID NO: 17. In some embodiments, the nucleic acid molecule encodes SEQ ID NO:5.

In some embodiments, the nucleic acid molecule comprises one or more nucleotide sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:43, SEQ ID NO:45, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55 or SEQ ID NO:56. In some embodiments, the nucleic acid molecule comprises a combination of a) SEQ ID NO:2 or SEQ ID NO:49, b) SEQ ID NO:4, c) SEQ ID NO:6 or SEQ ID NO:56, d) SEQ ID NO: 8 or SEQ ID NO:50, e) SEQ ID NO:10 or SEQ ID NO:51, f) SEQ ID NO:12 or SEQ ID NO:52, g) SEQ ID NO:14 or SEQ ID NO:53, h) SEQ ID NO:16, SEQ ID NO:43 or SEQ ID NO:54, and i) SEQ ID NO:18 or SEQ ID NO:55. In some embodiments, the nucleic acid molecule comprises a combination of a) SEQ ID NO:20, b) SEQ ID NO:22, c) SEQ ID NO:24 or SEQ ID NO:48, d) SEQ ID NO:26, and e) SEQ ID NO:28. In some embodiments, the nucleic acid molecule comprises a combination of SEQ ID NO:30, SEQ ID NO:32, SEQ ID NO:34, SEQ ID NO:36, SEQ ID NO:38, and SEQ ID NO:40. In some embodiments, the nucleic acid molecule comprises a combination of a) SEQ ID NO:42, b) SEQ ID NO:16, SEQ ID NO:43 or SEQ ID NO:54, c) SEQ ID NO:45, d) SEQ ID NO:47, and e) SEQ ID NO:24 or SEQ ID NO:48. In some embodiments, the nucleic acid molecule comprises a combination of SEQ ID NO:49, SEQ ID NO:50, SEQ ID NO:51, SEQ ID NO:52, SEQ ID NO:53, SEQ ID NO:54, or SEQ ID NO:55. In some embodiments, the nucleic acid molecule comprises SEQ ID NO:6 or SEQ ID:56.

In one embodiment, the invention includes nucleic acid molecules that encode an amino acid of: SEQ ID NO:57, SEQ ID NO:59 SEQ ID NO:61, SEQ ID NO:63, SEQ ID NO:65, or SEQ ID NO:67, a fragment thereof encoding at least one TB antigen, or a sequence encoding at least one TB antigen having at least 95% identity to at least one TB antigen.

In one embodiment, the invention includes nucleic acid molecules having a nucleotide sequences selected for the group consisting of: SEQ ID NO:58, SEQ ID NO:60, SEQ ID NO:62, SEQ ID NO:64, SEQ ID NO:66, or SEQ ID NO:68, or a fragment thereof encoding at least one TB antigen, or a sequence comprising at least 95% identity to at least one TB antigen coding sequence.

In some embodiments, the invention includes methods of inducing an immune response against TB in an individual.

In some embodiments, the invention includes methods of treating an individual who has been diagnosed with TB.

In some embodiments, the invention includes methods of preventing TB infection an individual.

### DETAILED DESCRIPTION

Safe, effective and economical TB vaccines are provided including embodiments employing nucleic acid vaccine technology. The TB vaccines may be used in methods that can induce immune responses against immunogenic TB antigens, protect against TB infection and provide effective treatment to individual who are infected with TB. DNA vaccine technology can be used to provide cost-effective delivery of TB vaccine to large populations of individuals, enabling mass prophylactic vaccination against TB.

In some embodiments, the nucleic acid molecules are provided which comprise one or more nucleotide sequences encoding one or more TB antigens. In some embodiments, the nucleic acid molecules encode one or more of EsxQ, EsxS, EsxR, EsxD, EsxO, EsxU, EsxV, EsxA, EsxB, PPE51, EspC, Rpf B, Rv2034c, Rv2628c, Rv2719c, Rv0010c, Rv1872c, Rv0012, Rv0090c, Rv0095, Ag85B, Rv1733c, or Rv2626c.

In some embodiments, multivalent nucleic acid molecules are provided which encode two or more TB antigens. In some embodiments, the nucleic acid molecules encode two or more of EsxQ, EsxS, EsxR, EsxD, EsxO, EsxU, EsxV, EsxA, EsxB, PPE51, EspC, Rpf B, Rv2034c, Rv2628c, Rv2719c, Rv0010c, Rv1872c, Rv0012, Rv0090c, Rv0095, Ag85B, Rv1733c, or Rv2626c.

In some embodiments, the nucleic acid molecule comprises nucleotide sequences that encodes EsxQ, EsxS, EsxR, EsxD, EsxO, EsxU, EsxV, EsxA, and/or EsxB. In some embodiments, the nucleic acid molecule comprises nucleotide sequences that encodes PPE51, EspC, Rpf B, Rv2034c, and/or Rv2628c. In some embodiments, the nucleic acid molecule comprises nucleotide sequences that encodes Rv2719c, Rv0010c, Rv1872c, Rv0012, Rv0090c, and/or Rv0095. In some embodiments, the nucleic acid molecule comprises nucleotide sequences that encodes Ag85B, EsxA, Rv1733c, Rv2626c, and/or Rpf B.

### Definitions

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

a. Adjuvant

“Adjuvant” as used herein may mean any molecule added to the DNA plasmid vaccines described herein to enhance antigenicity of the one or more TB antigens encoded by the DNA plasmids and encoding nucleic acid sequences described hereinafter.

b. Antibody

“Antibody” may mean an antibody of classes IgG, IgM, IgA, IgD or IgE, or fragments, fragments or derivatives thereof, including Fab, F(ab′)2, Fd, and single chain antibodies, diabodies, bispecific antibodies, bifunctional antibodies and derivatives thereof. The antibody may be an antibody isolated from the serum sample of mammal, a polyclonal antibody, affinity purified antibody, or mixtures thereof which exhibits sufficient binding specificity to a desired epitope or a sequence derived therefrom.

c. Coding Sequence

“Coding sequence” or “encoding nucleic acid” as used herein may mean refers to the nucleic acid (RNA or DNA molecule) that comprise a nucleotide sequence which encodes a protein. The coding sequence may further include initiation and termination signals operably linked to regulatory elements including a promoter and polyadenylation signal capable of directing expression in the cells of an individual or mammal to whom the nucleic acid is administered.

d. Complement

“Complement” or “complementary” as used herein may mean a nucleic acid may mean Watson-Crick (e.g., A-T/U and C-G) or Hoogsteen base pairing between nucleotides or nucleotide analogs of nucleic acid molecules.

e. Consensus or Consensus Sequence

“Consensus” or “consensus sequence” as used herein may mean a synthetic nucleic acid sequence, or corresponding polypeptide sequence, constructed based on analysis of an alignment of multiple subtypes of a particular TB antigen, that can be used to induce broad immunity against multiple subtypes or serotypes of a particular TB antigen. Consensus TB antigens may include consensus amino acid sequences of proteins of the esat-6 family as set forth herein. Nucleotide sequences that encode the consensus amino acid sequences are also provided. Also, synthetic antigens such as fusion proteins may be manipulated to include consensus sequences (or consensus antigens).

f. Constant Current

“Constant current” as used herein to define a current that is received or experienced by a tissue, or cells defining said tissue, over the duration of an electrical pulse delivered to same tissue. The electrical pulse is delivered from the electroporation devices described herein. This current remains at a constant amperage in said tissue over the life of an electrical pulse because the electroporation device provided herein has a feedback element, preferably having instantaneous feedback. The feedback element can measure the resistance of the tissue (or cells) throughout the duration of the pulse and cause the electroporation device to alter its electrical energy output (e.g., increase voltage) so current in same tissue remains constant throughout the electrical pulse (on the order of microseconds), and from pulse to pulse. In some embodiments, the feedback element comprises a controller.

g. Current Feedback or Feedback

“Current feedback” or “feedback” as used herein may be used interchangeably and may mean the active response of the provided electroporation devices, which comprises measuring the current in tissue between electrodes and altering the energy output delivered by the EP device accordingly in order to maintain the current at a constant level. This constant level is preset by a user prior to initiation of a pulse sequence or electrical treatment. The feedback may be accomplished by the electroporation component, e.g., controller, of the electroporation device, as the electrical circuit therein is able to continuously monitor the current in tissue between electrodes and compare that monitored current (or current within tissue) to a preset current and continuously make energy-output adjustments to maintain the monitored current at preset levels. The feedback loop may be instantaneous as it is an analog closed-loop feedback.

h. Decentralized Current

“Decentralized current” as used herein may mean the pattern of electrical currents delivered from the various needle electrode arrays of the electroporation devices described herein, wherein the patterns minimize, or preferably eliminate, the occurrence of electroporation related heat stress on any area of tissue being electroporated.

i. Electroporation

“Electroporation,” “electro-permeabilization,” or “electro-kinetic enhancement” (“EP”) as used interchangeably herein may refer to the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

j. Feedback Mechanism

“Feedback mechanism” as used herein may refer to a process performed by either software or hardware (or firmware), which process receives and compares the impedance of the desired tissue (before, during, and/or after the delivery of pulse of energy) with a present value, preferably current, and adjusts the pulse of energy delivered to achieve the preset value. A feedback mechanism may be performed by an analog closed loop circuit.

k. Fragment

“Fragment” may mean a polypeptide fragment of a TB antigen or polyprotein that is capable of eliciting an immune response in a mammal against TB by recognizing the particular TB antigen. A TB antigen may be one of the 23 members of the esat-6 protein family: esxA to esxW as well as TB antigens Ag85A and Ag85B, in each case with or without the IgE signal peptides, proteins 98% or more homologous to the consensus sequences set forth herein, proteins 99% or more homologous to the consensus sequences set forth herein, and proteins 100% identical to the consensus sequences set forth herein, in each case with or without signal peptides and/or a methionine at position 1. Fragments refer to less than full length of these proteins. A fragment may or may not for example comprise fragments of a TB Immunogen linked to a signal peptide such as an immunoglobulin signal peptide for example IgE signal peptide or IgG signal peptide.

“Fragment” may also mean a nucleic acid fragment of that encodes a TB antigen fragment set forth above.

l. Genetic Construct

“Genetic construct” s used herein refers to the DNA or RNA molecules that comprise a nucleotide sequence which encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

m. Homology

“Homology,” as used herein, refers to a degree of complementarity. There can be partial homology or complete homology (i.e., identity). A partially complementary sequence that at least partially inhibits a completely complementary sequence from hybridizing to a target nucleic acid is referred to using the functional term “substantially homologous.” When used in reference to a double-stranded nucleic acid sequence such as a cDNA or genomic clone, the term “substantially homologous,” as used herein, refers to a probe that can hybridize to a strand of the double-stranded nucleic acid sequence under conditions of low stringency. When used in reference to a single-stranded nucleic acid sequence, the term “substantially homologous,” as used herein, refers to a probe that can hybridize to (i.e., is the complement of) the single-stranded nucleic acid template sequence under conditions of low stringency.

n. Identical

“Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

o. Impedance

“Impedance” as used herein may be used when discussing the feedback mechanism and can be converted to a current value according to Ohm's law, thus enabling comparisons with the preset current.

p. Immune Response

“Immune response” as used herein may mean the activation of a host's immune system, e.g., that of a mammal, in response to the introduction of one or more TB antigens via the provided DNA plasmid vaccines. The immune response can be in the form of a cellular or humoral response, or both.

q. Nucleic Acid

“Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

r. Operably Linked

“Operably linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

s. Promoter

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

t. Signal Peptide

“Signal peptide” and “leader sequence” are used interchangeably herein and refer to an amino acid sequence that can be linked at the amino terminus of a protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

u. Stringent Hybridization Conditions

“Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence-dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

v. Substantially Complementary

“Substantially complementary” as used herein may mean that a first sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under stringent hybridization conditions.

w. Substantially Identical

“Substantially identical” as used herein may mean that a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or with respect to nucleic acids, if the first sequence is substantially complementary to the complement of the second sequence.

x. Subtype

“Subtype” or “serotype”: as used herein, interchangeably, and in reference to HBV, means genetic variants of an HBV such that one subtype is recognized by an immune system apart from a different subtype.

y. Variant

“Variant” used herein with respect to a nucleic acid may mean (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of 2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within +2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

z. Vector

“Vector” used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

### TB Antigens and Coding Sequences of TB Antigens

In some embodiments, the invention provides multivalent compositions for expression or administration of two or more TB antigen. Exemplary TB antigens that can be included in the multivalent compositions of the invention include, but are not limited to EsxQ, EsxS, EsxR, EsxD, EsxO, EsxU, EsxV, EsxA, EsxB, PPE51, EspC, Rpf B, Rv2034c, Rv2628c, Rv2719c, Rv0010c, Rv1872c, Rv0012, Rv0090c, Rv0095, Ag85B, Rv1733c, and/or Rv2626c or fragments or variants thereof. In some embodiments, the compositions comprise one or more TB antigen comprising an amino acid sequence as set forth in SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:44, and SEQ ID NO:46, or fragments or variants thereof.

In one embodiment, the compositions comprise a combination of nine TB antigens, EsxQ-EsxS-EsxR-EsxD-EsxO-EsxU-EsxV-EsxA-EsxB, as set forth SEQ ID NO: 1, SEQ ID NO:3, SEQ ID NO:5, SEQ ID NO:7, SEQ ID NO:9, SEQ ID NO: 11, SEQ ID NO:13, SEQ ID NO:15, and SEQ ID NO:17. In some embodiments, the TB antigens are encoded on a single polypeptide with a protease cleavage site between each encoded antigen. In some embodiments, the single polypeptide comprises an amino acid sequence as set forth in SEQ ID NO:57. The construct includes the optional IgE leader sequence at the N terminal (SEQ ID NO:69). It is intended that this construct be considered as two alternatives: one as shown with the IgE leader and one without it. In the latter case, a start codon may be provided in place of the sequence encoding IgE leader.

In one embodiment, the compositions comprise a combination of five TB antigens, PPE51-EspC-RpfB-Rv2034c-Rv2628, wherein the antigens comprise amino acid sequences as set forth SEQ ID NO:19, SEQ ID NO:21, SEQ ID NO:23, SEQ ID NO:25, and SEQ ID NO:27. In some embodiments, the TB antigens are encoded on a single polypeptide with a protease cleavage site between each encoded antigen. In some embodiments, the single polypeptide comprises an amino acid sequence as set forth in SEQ ID NO:59. The construct includes the optional IgE leader sequence at the N terminal (SEQ ID NO:69). It is intended that this construct be considered as two alternatives: one as shown with the IgE leader and one without it. In the latter case, a start codon may be provided in place of the sequence encoding IgE leader.

In one embodiment, the compositions comprise a combination of six TB antigens, Rv2719c-Rv0010c-Rv1872c-Rv0012-Rv0090c-Rv0095, wherein the antigens comprise amino acid sequences as set forth SEQ ID NO:29, SEQ ID NO:31, SEQ ID NO:33, SEQ ID NO:35, SEQ ID NO:37, and SEQ ID NO:39. In some embodiments, the TB antigens are encoded on a single polypeptide with a protease cleavage site between each encoded antigen. In some embodiments, the single polypeptide comprises an amino acid sequence as set forth in SEQ ID NO:61. The construct includes the optional IgE leader sequence at the N terminal (SEQ ID NO:69). It is intended that this construct be considered as two alternatives: one as shown with the IgE leader and one without it. In the latter case, a start codon may be provided in place of the sequence encoding IgE leader.

In one embodiment, the compositions comprise a combination of five TB antigens, Ag85B-EsxA-Rv1733c-Rv2626c-Rv1009c, wherein the antigens comprise amino acid sequences as set forth SEQ ID NO:41, SEQ ID NO:15, SEQ ID NO:44, SEQ ID NO:46, and SEQ ID NO:23. In some embodiments, the TB antigens are encoded on a single polypeptide with a protease cleavage site between each encoded antigen. In some embodiments, the single polypeptide comprises an amino acid sequence as set forth in SEQ ID NO:63. The construct includes the optional IgE leader sequence at the N terminal (SEQ ID NO:69). It is intended that this construct be considered as two alternatives: one as shown with the IgE leader and one without it. In the latter case, a start codon may be provided in place of the sequence encoding IgE leader.

In one embodiment, the compositions comprise a combination of seven TB antigens, EsxQ-EsxD-EsxO-EsxU-EsxV-EsA-EsxB, wherein the antigens comprise amino acid sequences as set forth SEQ ID NO:1, SEQ ID NO:7, SEQ ID NO:9, SEQ ID NO: 11, SEQ ID NO:13, SEQ ID NO:15, and SEQ ID NO:17. In some embodiments, the TB antigens are encoded on a single polypeptide with a protease cleavage site between each encoded antigen. In some embodiments, the single polypeptide comprises an amino acid sequence as set forth in SEQ ID NO:65. The construct includes the optional IgE leader sequence at the N terminal (SEQ ID NO:69). It is intended that this construct be considered as two alternatives: one as shown with the IgE leader and one without it. In the latter case, a start codon may be provided in place of the sequence encoding IgE leader.

A fragment of a TB protein may be a fragment or a variant of a TB antigen. Such fragments comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of a protein having 95% or more, 96% or more, 97% or more, 98% or more of 99% or more sequence identity to any one of SEQ ID NO: 1, SEQ ID NO:3, SEQ ID NO:5, SEQ ID NO:7, SEQ ID NO:9, SEQ ID NO: 11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:44, or SEQ ID NO:46.

In some embodiments, the compositions comprise one or more fragment of a TB antigen described herein. A fragment of a TB protein may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% of the full length of any one of SEQ ID NO: 1, SEQ ID NO:3, SEQ ID NO:5, SEQ ID NO:7, SEQ ID NO:9, SEQ ID NO: 1l, SEQ ID NO:13, SEQ ID NO:15, SEQ ID NO:17, SEQ ID NO: 19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:44, or SEQ ID NO:46.

In some embodiments, the compositions comprise one or more variant of a TB antigen described herein. A variant of a TB antigen may have 80% or more, 90% or more, 95% or more, 96% or more, 97% or more, 98% or more, or 99% or more identity to any one of SEQ ID NO:1, SEQ ID NO:3, SEQ ID NO:5, SEQ ID NO:7, SEQ ID NO:9, SEQ ID NO: 11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:44, or SEQ ID NO: 46.

In some embodiments, the compositions comprises a polypeptide molecule comprising multiple TB antigens separated by protease cleavage sites. In one embodiment the polypeptide molecule comprises SEQ ID NO:57, SEQ ID NO:59 SEQ ID NO:61, SEQ ID NO:63, SEQ ID NO:65, or SEQ ID NO:67 or a fragment or variant thereof.

In some embodiments, the fragment is a fragment of SEQ ID NO:57, SEQ ID NO:59 SEQ ID NO:61, SEQ ID NO:63, SEQ ID NO:65, or SEQ ID NO:67 lacking the IgE leader sequence (SEQ ID NO:69). A fragment of a TB polypeptide may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% of the full length of any one of SEQ ID NO:57, SEQ ID NO:59 SEQ ID NO:61, SEQ ID NO:63, SEQ ID NO:65, or SEQ ID NO:67.

In some embodiments, the variant is a variant of SEQ ID NO:57, SEQ ID NO:59 SEQ ID NO:61, SEQ ID NO:63, SEQ ID NO:65, or SEQ ID NO:67 lacking the IgE leader sequence (SEQ ID NO:69). A variant of a TB polypeptide may have 80% or more, 90% or more, 95% or more, 96% or more, 97% or more, 98% or more, or 99% or more identity to any one of SEQ ID NO:57, SEQ ID NO:59 SEQ ID NO:61, SEQ ID NO:63, SEQ ID NO:65, or SEQ ID NO:67.

### Nucleic Acid Molecules

Provided herein are nucleic acid molecules that are capable of expressing one or more TB antigen. In some embodiments, provided are multivalent TB constructs for expression of two or more TB antigens from a single nucleic acid molecule. The nucleic acid molecules may comprise heterologous nucleic acid encoding the one or more TB antigens. The nucleic acid molecules may comprise DNA molecules encoding the one or more TB antigens. The nucleic acid molecules may comprise RNA molecules (e.g., mRNA) encoding the one or more TB antigens. In one embodiment, the nucleic acid molecule may be a plasmid. The plasmid may be useful for transfecting cells with a nucleic acid encoding one or more TB antigen.

In some embodiments, nucleic acid molecule comprises a nucleotide sequence that encodes one TB antigen. In some embodiments, the nucleic acid molecule comprises a nucleotide sequence that encodes two TB antigens. In some embodiments, the nucleic acid molecule comprises a nucleotide sequence that encodes three TB antigens. In some embodiments, the nucleic acid molecule comprises a nucleotide sequence that encodes four TB antigens. In some embodiments, the nucleic acid molecule comprises a nucleotide sequence that encodes five TB antigens. In some embodiments, the nucleic acid molecule comprises a nucleotide sequence that encodes six TB antigens. In some embodiments, the nucleic acid molecule comprises a nucleotide sequence that encodes seven TB antigens. In some embodiments, the nucleic acid molecule comprises a nucleotide sequence that encodes eight TB antigens. In some embodiments, the nucleic acid molecule comprises a nucleotide sequence that encodes nine TB antigens. In some embodiments, the nucleic acid molecule comprises a nucleotide sequence that encodes ten TB antigens. In some embodiments, the nucleic acid molecule comprises a nucleotide sequence that encodes eleven TB antigens. In some embodiments, the nucleic acid molecule comprises a nucleotide sequence that encodes twelve TB antigens. In some embodiments, the nucleic acid molecule comprises a nucleotide sequence that encodes thirteen TB antigens. In some embodiments, the nucleic acid molecule comprises a nucleotide sequence that encodes fourteen TB antigens. In some embodiments, the nucleic acid molecule comprises a nucleotide sequence that encodes fifteen TB antigens. In some embodiments, the nucleic acid molecule comprises a nucleotide sequence that encodes sixteen TB antigens. In some embodiments, the nucleic acid molecule comprises a nucleotide sequence that encodes seventeen TB antigens. In some embodiments, the nucleic acid molecule comprises a nucleotide sequence that encodes eighteen TB antigens. In some embodiments, the nucleic acid molecule comprises a nucleotide sequence that encodes nineteen TB antigens. In some embodiments, the nucleic acid molecule comprises a nucleotide sequence that encodes twenty TB antigens. In some embodiments, the nucleic acid molecule comprises a nucleotide sequence that encodes twenty-one TB antigens. In some embodiments, the nucleic acid molecule comprises a nucleotide sequence that encodes twenty-two TB antigens. In some embodiments, the nucleic acid molecule comprises a nucleotide sequence that encodes twenty-three TB antigens. In some embodiments, the nucleic acid molecule comprises a nucleotide sequence that encodes more than twenty-three TB antigens.

In some embodiments, the nucleic acid molecule encodes one or more of: Rv3017c (EsxQ), Rv3020c (EsxS), Rv3019c (EsxR), Rv3891c (EsxD), Rv2346c (EsxO), Rv3445c (EsxU), Rv3619c (EsxV), Rv3875 (EsxA), Rv3874 (EsxB), Rv3136c (PPE51), Rv3615c (EspC), Rv1009c (Rpf B), Rv2034c, Rv2628c, Rv2719c, Rv0010c, Rv1872c, Rv0012, Rv0090c, Rv0095, Rv1886c (Ag85B), Rv1733c, or Rv2626c.

In some embodiments, the nucleic acid molecule comprises nucleotide sequences encoding a combination of EsxQ, EsxS, EsxR, EsxD, EsxO, EsxU, EsxV, EsxA, and EsxB tuberculosis antigens. In some embodiments, the nucleic acid molecule comprises nucleotide sequences encoding a combination of PPE51, EspC, Rpf B, Rv2034c, and Rv2628c tuberculosis antigens. In some embodiments, the nucleic acid molecule comprises nucleotide sequences encoding a combination of Rv2719c, Rv0010c, Rv1872c, Rv0012, Rv0090c, and Rv0095 tuberculosis antigens. In some embodiments, the nucleic acid molecule comprises nucleotide sequences encoding a combination of Ag85B, EsxA, Rv1733c, Rv2626c, and Rpf B tuberculosis antigens. In some embodiments, the nucleic acid molecule comprises nucleotide sequences encoding a combination of EsxQ, EsxD, EsxO, EsxU, EsxV, EsxA, and EsxB tuberculosis antigens. In some embodiments, the nucleic acid molecule comprises a nucleotide sequence encoding EsxR tuberculosis antigen.

In some embodiments, the nucleic acid molecule encodes one or more amino acid sequence of SEQ ID NO:1, SEQ ID NO:3, SEQ ID NO:5, SEQ ID NO:7, SEQ ID NO:9, SEQ ID NO: 11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:44, or SEQ ID NO:46, or a fragment or variant thereof. In some embodiments, the nucleic acid molecule encodes a combination of SEQ ID NO:1, SEQ ID NO:3, SEQ ID NO:5, SEQ ID NO:7, SEQ ID NO:9, SEQ ID NO: 11, SEQ ID NO:13, SEQ ID NO:15, and SEQ ID NO:17. In some embodiments, the nucleic acid molecule encodes a combination of SEQ ID NO: 19, SEQ ID NO:21, SEQ ID NO:23, SEQ ID NO:25, and SEQ ID NO:27. In some embodiments, the nucleic acid molecule encodes a combination of SEQ ID NO:29, SEQ ID NO:31, SEQ ID NO:33, SEQ ID NO:35, SEQ ID NO:37, and SEQ ID NO:39. In some embodiments, the nucleic acid molecule encodes a combination of SEQ ID NO:41, SEQ ID NO:15, SEQ ID NO:44, SEQ ID NO:46, and SEQ ID NO:23. In some embodiments, the nucleic acid molecule encodes a combination of SEQ ID NO:1, SEQ ID NO:7, SEQ ID NO:9, SEQ ID NO:11, SEQ ID NO:13, SEQ ID NO:15, and SEQ ID NO:17. In some embodiments, the nucleic acid molecule encodes SEQ ID NO:5.

In some embodiments, the nucleic acid molecule comprises one or more nucleotide sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:43, SEQ ID NO:45, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55 or SEQ ID NO:56, or a fragment or variant thereof. In some embodiments, the nucleic acid molecule comprises a combination of a) SEQ ID NO:2 or SEQ ID NO:49, b) SEQ ID NO:4, c) SEQ ID NO:6 or SEQ ID NO:56, d) SEQ ID NO: 8 or SEQ ID NO:50, e) SEQ ID NO:10 or SEQ ID NO:51, f) SEQ ID NO:12 or SEQ ID NO:52, g) SEQ ID NO:14 or SEQ ID NO:53, h) SEQ ID NO:16, SEQ ID NO:43 or SEQ ID NO:54, and i) SEQ ID NO:18 or SEQ ID NO:55, or a fragment or variant thereof. In some embodiments, the nucleic acid molecule comprises a combination of a) SEQ ID NO:20, b) SEQ ID NO:22, c) SEQ ID NO:24 or SEQ ID NO:48, d) SEQ ID NO:26, and e) SEQ ID NO:28, or a fragment or variant thereof. In some embodiments, the nucleic acid molecule comprises a combination of SEQ ID NO:30, SEQ ID NO:32, SEQ ID NO:34, SEQ ID NO:36, SEQ ID NO:38, and SEQ ID NO:40, or a fragment or variant thereof. In some embodiments, the nucleic acid molecule comprises a combination of a) SEQ ID NO:42, b) SEQ ID NO:16, SEQ ID NO:43 or SEQ ID NO:54, c) SEQ ID NO:45, d) SEQ ID NO:47, and e) SEQ ID NO:24 or SEQ ID NO: 48, or a fragment or variant thereof. In some embodiments, the nucleic acid molecule comprises a combination of SEQ ID NO:49, SEQ ID NO:50, SEQ ID NO:51, SEQ ID NO:52, SEQ ID NO:53, SEQ ID NO:54, or SEQ ID NO:55, or a fragment or variant thereof. In some embodiments, the nucleic acid molecule comprises SEQ ID NO:6 or SEQ ID:56, or a fragment or variant thereof.

In some embodiments, the nucleic acid molecule encodes a fragment or a variant of a TB antigen. Such fragments comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of a protein having 95% or more, 96% or more, 97% or more, 98% or more of 99% or more sequence identity to any one of SEQ ID NO:1, SEQ ID NO:3, SEQ ID NO:5, SEQ ID NO:7, SEQ ID NO:9, SEQ ID NO: 11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:44, or SEQ ID NO:46.

In some embodiments, the nucleic acid molecule encodes one or more fragment of a TB antigen described herein. A fragment of a TB protein may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% of the full length of any one of SEQ ID NO: 1, SEQ ID NO:3, SEQ ID NO:5, SEQ ID NO:7, SEQ ID NO:9, SEQ ID NO: 11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:44, or SEQ ID NO:46.

In some embodiments, the nucleic acid molecule encodes one or more variant of a TB antigen described herein. A variant of a TB antigen may have 80% or more, 90% or more, 95% or more, 96% or more, 97% or more, 98% or more, or 99% or more identity to any one of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:44, or SEQ ID NO:46.

In some embodiments, the nucleic acid molecule comprises a nucleotide sequence that has 80% or more, 90% or more, 95% or more, 96% or more, 97% or more, 98% or more, or 99% or more identity to any one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:43, SEQ ID NO:45, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55 or SEQ ID NO:56.

In some embodiments, the nucleic acid molecule comprises 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% of the full length of any one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:43, SEQ ID NO:45, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55 or SEQ ID NO:56.

In some embodiments, the nucleic acid molecule comprises 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of a nucleotide sequence having 95% or more, 96% or more, 97% or more, 98% or more of 99% or more sequence identity to any one of SEQ ID NO:2, SEQ ID NO:4, SEQ ID NO:6, SEQ ID NO:8, SEQ ID NO:10, SEQ ID NO:12, SEQ ID NO:14, SEQ ID NO: 16, SEQ ID NO: 18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:43, SEQ ID NO:45, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55 or SEQ ID NO:56.

In one embodiment, the invention includes nucleic acid molecules that encode an amino acid of: SEQ ID NO:57, SEQ ID NO:59 SEQ ID NO:61, SEQ ID NO:63, SEQ ID NO:65, or SEQ ID NO:67, a fragment thereof encoding at least one TB antigen, or a sequence encoding at least one TB antigen having at least 95% identity to at least one TB antigen. In some embodiments, the fragment comprises a fragment of SEQ ID NO:57, SEQ ID NO:59 SEQ ID NO:61, SEQ ID NO:63, SEQ ID NO:65, or SEQ ID NO:67 lacking the IgE leader sequence (SEQ ID NO:69), the initial methionine, or any combination thereof.

In some embodiments, the invention includes nucleic acid molecules that encode a fragment of an amino acid of: SEQ ID NO:57, SEQ ID NO:59 SEQ ID NO:61, SEQ ID NO:63, SEQ ID NO:65, or SEQ ID NO:67. A fragment of may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% of the full length of any one of SEQ ID NO:57, SEQ ID NO:59 SEQ ID NO:61, SEQ ID NO:63, SEQ ID NO:65, or SEQ ID NO:67.

In some embodiments, the invention includes nucleic acid molecules that encode a variant of an amino acid of: SEQ ID NO:57, SEQ ID NO:59 SEQ ID NO:61, SEQ ID NO:63, SEQ ID NO:65, or SEQ ID NO:67. A variant of may have 80% or more, 90% or more, 95% or more, 96% or more, 97% or more, 98% or more, or 99% or more identity to any one of SEQ ID NO:57, SEQ ID NO:59 SEQ ID NO:61, SEQ ID NO:63, SEQ ID NO:65, or SEQ ID NO:67.

In one embodiment, the invention includes nucleic acid molecules having a nucleotide sequence of SEQ ID NO:58, SEQ ID NO:60, SEQ ID NO:62, SEQ ID NO:64, SEQ ID NO:66, or SEQ ID NO:68, or a fragment or variant thereof encoding at least one TB antigen, or a sequence comprising at least 95% identity to at least one TB antigen coding sequence. In some embodiments, the fragment comprises a fragment of SEQ ID NO:58, SEQ ID NO:60, SEQ ID NO:62, SEQ ID NO:64, SEQ ID NO:66, or SEQ ID NO:68 lacking the sequence encoding the IgE leader sequence (SEQ ID NO:69), one or both stop codons, the initial start codon, or any combination thereof.

In some embodiments, the invention includes a fragment of a nucleic acid molecule that has a nucleotide sequence of SEQ ID NO:58, SEQ ID NO:60, SEQ ID NO:62, SEQ ID NO:64, SEQ ID NO:66, or SEQ ID NO:68 lacking the nucleotide sequence encoding for the IgE leader sequence (SEQ ID NO:69). A fragment may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% of the full length of any one of SEQ ID NO:58, SEQ ID NO:60, SEQ ID NO:62, SEQ ID NO:64, SEQ ID NO:66, or SEQ ID NO:68 lacking the nucleotide sequence encoding for the IgE leader sequence (SEQ ID NO:69).

In some embodiments, the invention includes a variant of a nucleic acid molecule that has a nucleotide sequence of SEQ ID NO:58, SEQ ID NO:60, SEQ ID NO:62, SEQ ID NO:64, SEQ ID NO:66, or SEQ ID NO:68 lacking the nucleotide sequence encoding for the IgE leader sequence (SEQ ID NO:69). A variant of may have 80% or more, 90% or more, 95% or more, 96% or more, 97% or more, 98% or more, or 99% or more identity to any one of of SEQ ID NO:58, SEQ ID NO:60, SEQ ID NO:62, SEQ ID NO:64, SEQ ID NO:66, or SEQ ID NO:68 lacking the nucleotide sequence encoding for the IgE leader sequence (SEQ ID NO:69).

Table 1 shows example constructs that can be used in vaccines which can prevent TB infection and treat individuals infected with TB.

TABLE 1
Vector Design

| Vector | Design | SEQ ID NO: (AA/DNA) |
|---|---|---|
| pEsx | EsxQ-EsxS-EsxR-EsxD-EsxO-EsxU-EsxV-EsxA-EsxB | SEQ ID NO: 57/58 |
| P5.2Ag | PPE51-EspC-RpfB-Rv2034c-Rv2628 | SEQ ID NO: 59/60 |
| pVariable | Rv2719c-Rv0010c-Rv1872c-Rv0012-Rv0090c-Rv0095 | SEQ ID NO: 61/62 |
| P5Ag | Ag85B-EsxA-Rv1733c-Rv2626c-Rv1009c | SEQ ID NO: 63/64 |
| pEsx_R/S Null | EsxQ-EsxD-EsxO-EsxU-EsxV-EsA-EsxB | SEQ ID NO: 65/66 |
| pEsxR | Rv3019c | SEQ ID NO: 67/68 |

In some embodiments, the constructs of the invention can have an IgE signal peptide at the N terminus. The IgE signal peptide is optional, and it is intended that this disclosure be understood to be expressly disclosing sequences that include the IgE signal peptide at the N terminal and also expressly disclosing sequences that exclude the IgE signal peptide with either no residue or a N terminal Methionine or a site for accepting addition of a signal peptides from another protein.

In some embodiments, the invention provides multi-valent constructs encoding two or more TB antigens. In some embodiments, two or more TB antigens are separated by protease cleavage sites (e.g. furin cleavage sites). Any protease cleavage site which is processed by a protease commonly present in the cells of the vaccinated individual may be used in place of the furin sites.

The constructs may be rearranged of otherwise changed whether by changing the encoded antigens, the order of the encoded antigens, the number of encoded antigens, or any combination thereof on a given plasmid.

The nucleic acid molecule may further comprise an initiation codon, which may be upstream of the coding sequence, and a stop codon, which may be downstream of the coding sequence. The initiation and termination codon may be in frame with the coding sequence.

The genetic constructs can comprise regulatory elements for gene expression of the coding sequences of the nucleic acid. The regulatory elements can be a promoter, an enhancer an initiation codon, a stop codon, or a polyadenylation signal.

The nucleic acid sequences can make up a genetic construct that can be a vector. The vector can be capable of expressing an antigen in the cell of a mammal in a quantity effective to elicit an immune response in the mammal. The vector can be recombinant. The vector can comprise heterologous nucleic acid encoding the antigen. The vector can be a plasmid. The vector can be useful for transfecting cells with nucleic acid encoding an antigen, which the transformed host cell is cultured and maintained under conditions wherein expression of the antigen takes place.

Coding sequences can be optimized for stability and high levels of expression. In some instances, codons are selected to reduce secondary structure formation of the RNA such as that formed due to intermolecular bonding.

The nucleic acid molecule may also comprise a promoter that is operably linked to the coding sequence The promoter operably linked to the coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human actin, human myosin, human hemoglobin, human muscle creatine, or human metallothionein. The promoter may also be a tissue specific promoter, such as a muscle or skin specific promoter, natural or synthetic. Examples of such promoters are described in US patent application publication no. US20040175727, the contents of which are incorporated herein in its entirety.

The nucleic acid molecule may also comprise a polyadenylation signal, which may be downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human 3-globin polyadenylation signal. The SV40 polyadenylation signal may be a polyadenylation signal from a pCEP4 plasmid (Invitrogen, San Diego, CA).

The nucleic acid molecule may also comprise an enhancer upstream of the coding sequence. The enhancer may be human actin, human myosin, human hemoglobin, human muscle creatine or a viral enhancer such as one from CMV, FMDV, RSV or EBV. Polynucleotide function enhances are described in U.S. Pat. Nos. 5,593,972, 5,962,428, and WO94/016737, the contents of each are fully incorporated by reference.

The nucleic acid molecule may also comprise a mammalian origin of replication in order to maintain the nucleic acid molecule extrachromosomally and produce multiple copies of the nucleic acid molecule in a cell. The nucleic acid molecule may be pVAX1, pCEP4 or pREP4 from Invitrogen (San Diego, CA), which may comprise the Epstein Barr virus origin of replication and nuclear antigen EBNA-1 coding region, which may produce high copy episomal replication without integration.

The vector can be pVAX1 or a pVax1 variant with changes such as the variant nucleic acid molecule described herein. The variant pVax1 plasmid is a 2998 basepair variant of the backbone vector plasmid pVAX1 (Invitrogen, Carlsbad CA). The CMV promoter is located at bases 137-724. The T7 promoter/priming site is at bases 664-683. Multiple cloning sites are at bases 696-811. Bovine GH polyadenylation signal is at bases 829-1053. The Kanamycin resistance gene is at bases 1226-2020. The pUC origin is at bases 2320-2993.

Based upon the sequence of pVAX1 available from Invitrogen, the following mutations were found in the sequence of pVAX1 that was used as the backbone plasmid for the nucleic acid constructs set forth herein:
C>G241 in CMV promoter
C>T 1942 backbone, downstream of the bovine growth hormone polyadenylation signal (bGHpolyA)
A>-2876 backbone, downstream of the Kanamycin gene
C>T 3277 in pUC origin of replication (Ori) high copy number mutation (see Nucleic Acid Research 1985)
G>C 3753 in very end of pUC Ori upstream of RNASeH site

Base pairs 2, 3 and 4 are changed from ACT to CTG in backbone, upstream of CMV promoter.

The backbone of the vector can be pAV0242. The vector can be a replication defective adenovirus type 5 (Ad5) vector.

The nucleic acid molecule may also comprise a regulatory sequence, which may be well suited for gene expression in a cell into which the nucleic acid molecule is administered. The coding sequence may comprise a codon that may allow more efficient transcription of the coding sequence in the host cell.

The coding sequence may also comprise an Ig leader sequence. The leader sequence may be 5′ of the coding sequence. The consensus antigens encoded by this sequence may comprise an N-terminal Ig leader followed by a consensus antigen protein. The N-terminal Ig leader may be IgE or IgG.

In one embodiment, the nucleic acid constructs are inserted into a plasmid backbone. The plasmid may be pSE420 (Invitrogen, San Diego, Calif.), which may be used for protein production in Escherichia coli (E. coli). The plasmid may also be pYES2 (Invitrogen, San Diego, Calif.), which may be used for protein production in Saccharomyces cerevisiae strains of yeast. The plasmid may also be of the MAXBAC™ complete baculovirus expression system (Invitrogen, San Diego, Calif.), which may be used for protein production in insect cells. The plasmid may also be pcDNA I or pcDNA3 (Invitrogen, San Diego, Calif), which may be used for protein production in mammalian cells such as Chinese hamster ovary (CHO) cells.

### Pharmaceutical Compositions and Vaccines

Compositions are provided which comprise the TB antigens or nucleic acid molecules encoding the same of the invention. For example, compositions may comprise a plurality of two, three, four five, six, seven, eight, nine, ten or more different TB antigens.

Compositions may comprise multi-valent nucleic acid molecules encoding a plurality of two, three, four five, six, seven, eight, nine, ten or more different TB antigens.

In some embodiments, the compositions are pharmaceutical compositions (e.g., a vaccine) capable of generating in a mammal an immune response against TB. The vaccine may comprise a combination of TB antigens as discussed above. The immunogenic composition may comprise a multi-valent nucleic acid molecule encoding a combination of TB antigens as discussed above. The immunogenic composition may comprise a plurality of nucleic acid molecules encoding one or more TB antigens as discussed above. The immunogenic composition may comprise a plurality of multi-valent nucleic acid molecules, wherein each nucleic acid molecule encodes a combination of TB antigens as discussed above. The immunogenic composition may be provided to induce a therapeutic or prophylactic immune response.

The immunogenic composition can be in the form of a pharmaceutical composition. The pharmaceutical composition can comprise a vaccine.

The immunogenic composition may comprise the combination of TB antigens or one or more nucleic acid molecule at quantities of from about 1 nanogram to 100 milligrams; about 1 microgram to about 10 milligrams; or preferably about 0.1 microgram to about 10 milligrams; or more preferably about 1 milligram to about 2 milligram. In some preferred embodiments, pharmaceutical compositions according to the present invention comprise about 5 nanogram to about 1000 micrograms of RNA or DNA. In some preferred embodiments, the pharmaceutical compositions contain about 10 nanograms to about 800 micrograms of RNA or DNA. In some preferred embodiments, the pharmaceutical compositions contain about 25 to about 250 micrograms, from about 100 to about 200 microgram, from about 1 nanogram to 100 milligrams; from about 1 microgram to about 10 milligrams; from about 0.1 microgram to about 10 milligrams; from about 1 milligram to about 2 milligram, from about 5 nanogram to about 1000 micrograms, from about 10 nanograms to about 800 micrograms, from about 0.1 to about 500 micrograms, from about 1 to about 350 micrograms, from about 25 to about 250 micrograms, from about 100 to about 200 microgram of the consensus antigen or plasmid thereof. The pharmaceutical compositions can comprise about 5 nanograms to about 10 mg of one or more nucleic acid molecule. In some embodiments, pharmaceutical compositions according to the present invention comprise about 25 nanogram to about 5 mg of one or more nucleic acid molecule. In some embodiments, the pharmaceutical compositions contain about 50 nanograms to about 1 mg of RNA or DNA. In some embodiments, the pharmaceutical compositions contain about 0.1 to about 500 micrograms of RNA or DNA. In some embodiments, the pharmaceutical compositions contain about 1 to about 350 micrograms of RNA or DNA. In some embodiments, the pharmaceutical compositions contain about 5 to about 250 micrograms of RNA or DNA. In some embodiments, the pharmaceutical compositions contain about 10 to about 200 micrograms of RNA or DNA. In some embodiments, the pharmaceutical compositions contain about 15 to about 150 micrograms of RNA or DNA. In some embodiments, the pharmaceutical compositions contain about 20 to about 100 micrograms of RNA or DNA. In some embodiments, the pharmaceutical compositions contain about 25 to about 75 micrograms of RNA or DNA. In some embodiments, the pharmaceutical compositions contain about 30 to about 50 micrograms of RNA or DNA. In some embodiments, the pharmaceutical compositions contain about 35 to about 40 micrograms of RNA or DNA. In some embodiments, the pharmaceutical compositions contain about 100 to about 200 microgram RNA or DNA. In some embodiments, the pharmaceutical compositions comprise about 10 microgram to about 100 micrograms of RNA or DNA. In some embodiments, the pharmaceutical compositions comprise about 20 micrograms to about 80 micrograms of RNA or DNA. In some embodiments, the pharmaceutical compositions comprise about 25 micrograms to about 60 micrograms of RNA or DNA. In some embodiments, the pharmaceutical compositions comprise about 30 nanograms to about 50 micrograms of RNA or DNA. In some embodiments, the pharmaceutical compositions comprise about 35 nanograms to about 45 micrograms of RNA or DNA. In some preferred embodiments, the pharmaceutical compositions contain about 0.1 to about 500 micrograms of RNA or DNA. In some preferred embodiments, the pharmaceutical compositions contain about 1 to about 350 micrograms of RNA or DNA. In some preferred embodiments, the pharmaceutical compositions contain about 25 to about 250 micrograms of RNA or DNA. In some preferred embodiments, the pharmaceutical compositions contain about 100 to about 200 microgram RNA or DNA.

In some embodiments, pharmaceutical compositions according to the present invention comprise at least 10, 15, 20, 25, 30, 35, 40, 45, 50, 55, 60, 65, 70, 75, 80, 85, 90, 95 or 100 nanograms of RNA or DNA. In some embodiments, the pharmaceutical compositions can comprise at least 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895. 900, 905, 910, 915, 920, 925, 930, 935, 940, 945, 950, 955, 960, 965, 970, 975, 980, 985, 990, 995 or 1000 micrograms of RNA or DNA. In some embodiments, the pharmaceutical composition can comprise at least 1.5, 2, 2.5, 3, 3.5, 4, 4.5, 5, 5.5, 6, 6.5, 7, 7.5, 8, 8.5, 9, 9.5 or 10 mg or more of RNA or DNA.

In other embodiments, the pharmaceutical composition can comprise up to and including 15, 20, 25, 30, 35, 40, 45, 50, 55, 60, 65, 70, 75, 80, 85, 90, 95 or 100 nanograms of RNA or DNA. In some embodiments, the pharmaceutical composition can comprise up to and including 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895. 900, 905, 910, 915, 920, 925, 930, 935, 940, 945, 950, 955, 960, 965, 970, 975, 980, 985, 990, 995, or 1000 micrograms of RNA or DNA. In some embodiments, the pharmaceutical composition can comprise up to and including 1.5, 2, 2.5, 3, 3.5, 4, 4.5, 5, 5.5, 6, 6.5, 7, 7.5, 8, 8.5, 9, 9.5 or 10 mg of RNA or DNA.

The pharmaceutical composition can further comprise other agents for formulation purposes according to the mode of administration to be used. In cases where pharmaceutical compositions are injectable pharmaceutical compositions, they are sterile, pyrogen free and particulate free. An isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The vaccine can further comprise a pharmaceutically acceptable excipient. The pharmaceutically acceptable excipient can be functional molecules as vehicles, adjuvants, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and more preferably, the poly-L-glutamate is present in the vaccine at a concentration less than 6 mg/ml. The transfection facilitating agent can also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid can also be used administered in conjunction with the genetic construct. In some embodiments, the RNA or DNA vector vaccines can also include a transfection facilitating agent such as lipids, liposomes, including lecithin liposomes or other liposomes known in the art, as a RNA-liposome mixture or DNA-liposome mixture, calcium ions, viral proteins, polyanions, polycations, or nanoparticles, or other known transfection facilitating agents. Preferably, the transfection facilitating agent is a polyanion, polycation, including poly-L-glutamate (LGS), or lipid. Concentration of the transfection agent in the vaccine is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The pharmaceutically acceptable excipient can be an adjuvant. The adjuvant can be other genes that are expressed in alternative plasmid or are deneurological systemed as proteins in combination with the plasmid above in the vaccine. The adjuvant can be selected from the group consisting of: α-interferon (IFN-α), β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. The adjuvant can be IL-12, IL-15, IL-28, CTACK, TECK, platelet derived growth factor (PDGF), TNFα, TNFβ, GM-CSF, epidermal growth factor (EGF), IL-1, IL-2, IL-4, IL-5, IL-6, IL-10, IL-12, IL-18, or a combination thereof. In an exemplary embodiment, the adjuvant is IL-12.

Other genes which can be useful adjuvants include those encoding: MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, INK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof or a combination thereof.

The vaccine may further comprise a genetic vaccine facilitator agent as described in U.S. Ser. No. 021,579 filed Apr. 1, 1994, which is fully incorporated by reference.

The vaccine may be formulated according to the mode of administration to be used. An injectable vaccine pharmaceutical composition may be sterile, pyrogen free and particulate free. An isotonic formulation or solution may be used. Additives for isotonicity may include sodium chloride, dextrose, mannitol, sorbitol, and lactose. The vaccine may comprise a vasoconstriction agent. The isotonic solutions may include phosphate buffered saline. Vaccine may further comprise stabilizers including gelatin and albumin. The stabilizing may allow the formulation to be stable at room or ambient temperature for extended periods of time such as LGS or polycations or polyanions to the vaccine formulation.

The vaccine can be a DNA vaccine. The DNA vaccine can further comprise elements or reagents that inhibit it from integrating into the chromosome.

The vaccine can be an RNA vaccine. The RNA vaccine can further comprise elements or reagents that promote stability, increase translation, prevent degradation or increase the half-life of the RNA molecule.

The nucleic acid molecule can also be incorporated into a recombinant viral vector, including recombinant adenovirus, recombinant adenovirus associated virus and recombinant vaccinia. The genetic construct can be part of the genetic material in attenuated live microorganisms or recombinant microbial vectors which live in cells.

Examples of attenuated live vaccines, those using recombinant vectors to deliver foreign antigens, subunit vaccines and glycoprotein vaccines are described in U.S. Pat. Nos. 4,510,245; 4,797,368; 4,722,848; 4,790,987; 4,920,209; 5,017,487; 5,077,044; 5,110,587; 5,112,749; 5,174,993; 5,223,424; 5,225,336; 5,240,703; 5,242,829; 5,294,441; 5,294,548; 5,310,668; 5,387,744; 5,389,368; 5,424,065; 5,451,499; 5,453,364; 5,462,734; 5,470,734; 5,474,935; 5,482,713; 5,591,439; 5,643,579; 5,650,309; 5,698,202; 5,955,088; 6,034,298; 6,042,836; 6,156,319 and 6,589,529, which are each incorporated herein by reference.

### Nanoparticle Formulations

In one embodiment, the immunogenic composition of the invention may comprise a nanoparticle, including but not limited to a lipid nanoparticle (LNP), comprising a multi-valent nucleic acid molecule encoding two or more TB antigens of the invention. In some embodiments, the composition comprises an mRNA molecule that encodes two or more TB antigens of the invention.

In one embodiment, the LNP comprises or encapsulates an RNA molecule encoding at least two amino acid sequence of, SEQ ID NO:1, SEQ ID NO:3, SEQ ID NO:5, SEQ ID NO:7, SEQ ID NO:9, SEQ ID NO: 11, SEQ ID NO:13, SEQ ID NO:15, SEQ ID NO:17, SEQ ID NO: 19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:44, or SEQ ID NO:46 or a fragment or variant thereof.

In one embodiment, the LNP comprises or encapsulates an RNA molecule comprising at least two RNA sequences corresponding to the DNA sequences as set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, EQ ID NO:30, SEQ ID NO:32, SEQ ID NO:34, SEQ ID NO:36, SEQ ID NO:38, SEQ ID NO:40, SEQ ID NO:42, SEQ ID NO:43, SEQ ID NO:45, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, or SEQ ID NO:56, or a fragment or variant thereof.

In one embodiment, the LNP comprises or encapsulates an RNA molecule comprising an RNA sequence corresponding to a DNA sequence as set forth in SEQ ID NO:58, SEQ ID NO:60, SEQ ID NO:62, SEQ ID NO:64, SEQ ID NO:66, or SEQ ID NO:68.

### Methods of Treatment

Also provided herein is a method of treating, protecting against, and/or preventing disease in a subject in need thereof by administering the vaccine to the subject. Administration of the vaccine to the subject can induce or elicit an immune response in the subject. The induced immune response can be used to treat, prevent, and/or protect against disease, for example, pathologies relating to Mycobacterium tuberculosis infection. In some embodiments, the invention provides methods for treating or preventing tuberculosis or a disease or disorder associated with Mycobacterium tuberculosis infection in a subject in need thereof. In some embodiments, the methods include administering a combination of TB antigens as described herein, a nucleic acid molecule encoding a combination of TB antigens as described herein, or a composition comprising a nucleic acid molecule encoding a combination of TB antigens as described herein to a subject in need thereof.

In some embodiments, administration of a combination of TB antigens as described herein, a nucleic acid molecule encoding a combination of TB antigens as described herein, or a composition comprising a nucleic acid molecule encoding a combination of TB antigens as described herein may induce an immune response against TB. In some embodiments, the immune response may be a therapeutic or prophylactic immune response.

In some embodiments, upon transfection of a multi-valent nucleic acid molecule of the invention into a cell, the transfected cells will express and secrete multiple TB antigens as the single encoded polypeptide is cleaved into multiple TB antigens by a protease present in the cell. These proteins will be recognized as foreign by the immune system and antibodies will be made against them. These antibodies will be maintained by the immune system and allow for an effective response to subsequent TB infections.

Methods of delivering DNA molecules are described in U.S. Pat. Nos. 4,945,050 and 5,036,006, both of which are incorporated herein in their entirety by reference.

The compositions of the invention may be administered to a mammal to elicit an immune response in a mammal. The mammal may be human, primate, non-human primate, cow, cattle, sheep, goat, antelope, bison, water buffalo, bison, bovids, deer, hedgehogs, elephants, llama, alpaca, mice, rats, and chicken.

The compositions of the invention can be used to generate an immune response in a mammal, including therapeutic or prophylactic immune response. The immune response can generate antibodies and/or killer T cells which are directed to the one or more TB antigens. Such antibodies and T cells can be isolated.

Some embodiments provide methods of generating immune responses against one or more TB antigens, which comprise administering to an individual the compositions of the invention. Some embodiments provide methods of prophylactically vaccinating an individual against TB infection, which comprise administering one or more composition of the invention. Some embodiments provide methods of therapeutically vaccinating an individual that has been infected with TB which comprise administering one or more composition of the invention. Diagnosis of TB infection prior to administration of the vaccine can be done routinely.

In some embodiments, administration of a composition of the invention induces humoral immunogenicity and provides protection from tuberculosis, providing 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% protection.

### Administration

The composition of the invention may be administered alone or in combination with other proteins, nucleic acid molecules or therapeutic molecules.

The composition of the invention may be administered by any appropriate route including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof.

For medical use, the composition may be administered as a suitably acceptable formulation in accordance with normal medical practice. The physician can readily determine the dosing regimen and route of administration that is most appropriate for a particular subject. For veterinary use, the composition may be administered as a suitably acceptable formulation in accordance with normal veterinary practice. The veterinarian can readily determine the dosing regimen and route of administration that is most appropriate for a particular animal.

The present invention also relates to a method of delivering the composition to the subject in need thereof. The method of delivery can include, administering the composition to the subject. Administration can include, but is not limited to, DNA injection with and without in vivo electroporation, liposome mediated delivery, and nanoparticle facilitated delivery.

The vaccine can be formulated in accordance with standard techniques well known to those skilled in the pharmaceutical art. Such compositions can be administered in dosages and by techniques well known to those skilled in the medical arts taking into consideration such factors as the age, sex, weight, and condition of the particular subject, and the route of administration. The subject can be a mammal, such as a human, a horse, a cow, a pig, a sheep, a cat, a dog, a rat, or a mouse.

The vaccine can be administered prophylactically or therapeutically. In prophylactic administration, the vaccines can be administered in an amount sufficient to induce an immune response. In therapeutic applications, the vaccines are administered to a subject in need thereof in an amount sufficient to elicit a therapeutic effect. An amount adequate to accomplish this is defined as “therapeutically effective dose.” Amounts effective for this use will depend on, e.g., the particular composition of the vaccine regimen administered, the manner of administration, the stage and severity of the disease, the general state of health of the patient, and the judgment of the prescribing physician.

The vaccine can be administered by methods well known in the art as described in Donnelly et al. (Ann. Rev. Immunol. 15:617-648 (1997)); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997), the contents of all of which are incorporated herein by reference in their entirety. The DNA of the vaccine can be complexed to particles or beads that can be administered to an individual, for example, using a vaccine gun. One skilled in the art would know that the choice of a pharmaceutically acceptable carrier, including a physiologically acceptable compound, depends, for example, on the route of administration of the expression vector.

The vaccine can be delivered via a variety of routes. Typical delivery routes include parenteral administration, e.g., intradermal, intramuscular or subcutaneous delivery. Other routes include oral administration, intranasal, and intravaginal routes. For the DNA of the vaccine in particular, the vaccine can be delivered to the interstitial spaces of tissues of an individual (Felgner et al., U.S. Pat. Nos. 5,580,859 and 5,703,055, the contents of all of which are incorporated herein by reference in their entirety). The vaccine can also be administered to muscle, or can be administered via intradermal or subcutaneous injections, or transdermally, such as by iontophoresis. Epidermal administration of the vaccine can also be employed. Epidermal administration can involve mechanically or chemically irritating the outermost layer of epidermis to stimulate an immune response to the irritant (Carson et al., U.S. Pat. No. 5,679,647, the contents of which are incorporated herein by reference in its entirety).

The vaccine can also be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the vaccine.

The vaccine can be a liquid preparation such as a suspension, syrup or elixir. The vaccine can also be a preparation for parenteral, subcutaneous, intradermal, intramuscular or intravenous administration (e.g., injectable administration), such as a sterile suspension or emulsion.

Nucleic acid molecules of the invention can be incorporated into liposomes, microspheres or other polymer matrices (Felgner et al., U.S. Pat. No. 5,703,055; Gregoriadis, Liposome Technology, Vols. I to III (2nd ed. 1993), the contents of which are incorporated herein by reference in their entirety). Liposomes can consist of phospholipids or other lipids, and can be nontoxic, physiologically acceptable and metabolizable carriers that are relatively simple to make and administer.

The vaccine can be administered via electroporation, such as by a method described in U.S. Pat. No. 7,664,545, the contents of which are incorporated herein by reference. The electroporation can be by a method and/or apparatus described in U.S. Pat. Nos. 6,302,874; 5,676,646; 6,241,701; 6,233,482; 6,216,034; 6,208,893; 6,192,270; 6,181,964; 6,150,148; 6,120,493; 6,096,020; 6,068,650; and 5,702,359, the contents of which are incorporated herein by reference in their entirety. The electroporation may be carried out via a minimally invasive device.

### EXAMPLES

The present invention is further illustrated in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims

### Example 1: Multivalent TB Vaccine

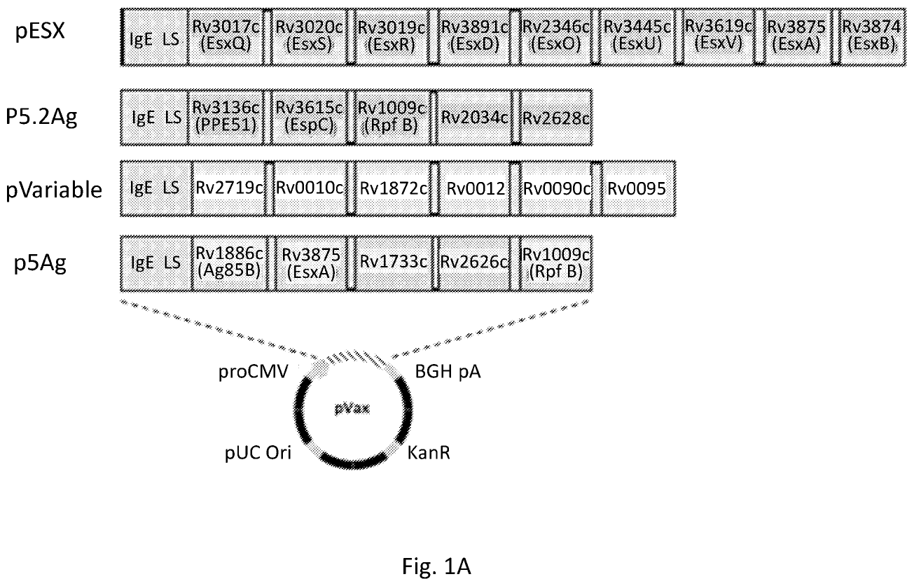

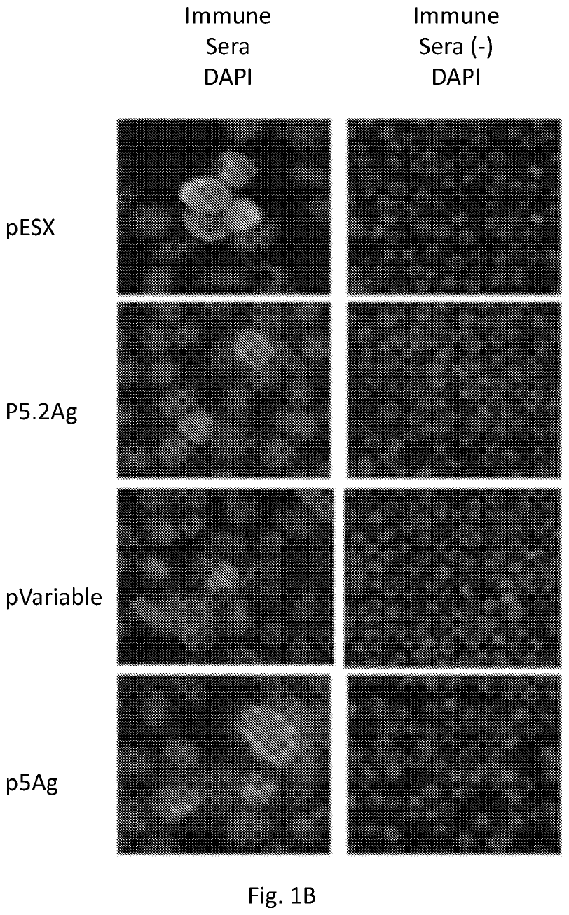

Four multivalent TB constructs were initially made (pESX, p5.2Ag, pVariable, and p5Ag). Synthetic DNA inserts were designed and synthesized containing an N-terminal IgE leader sequence, followed by the codon optimized sequences encoding the indicated M tb antigens (Rv strain) separated by furin cleavage sites (see FIG. 1A). These inserts were cloned into a pVax vector between the CMV-promoter (proCMV) and poly-adenosine sites (BGH pA).

All multivalent vectors were separated by endoproteolytic (furin) cleavage sites, which will allow for the secretion of each individual protein. Furthermore, the constructs were synthetically designed and codon and RNA optimized to improve expression. Sequences were synthesized into a desired vector that contained a kozak consensus sequence and IgE leader sequence at the 5′ end, to help enhance both protein efficiency and synthesis, and a poly A tail to end translation.

Prior to immunogenicity studies in mice, experiments were conducted to confirm antigen expression for the constructs. Cell lines were transfected with the indicated plasmids. FIG. 1B shows expression of vaccine antigens was detected by IFA using mouse antisera raised against the indicated constructs (left) compared to control sera (right). Mtb-specific antibodies were detected using FITC-labeled, anti-mouse (IgG) and nuclei were visualized by DAPI.

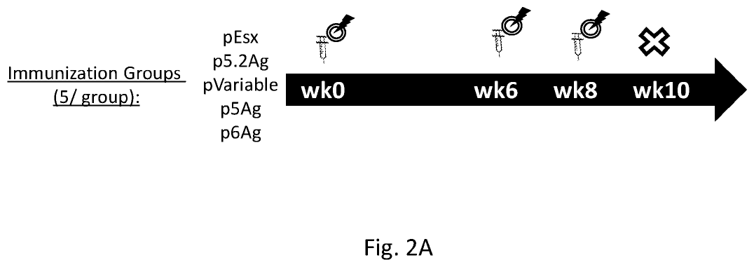

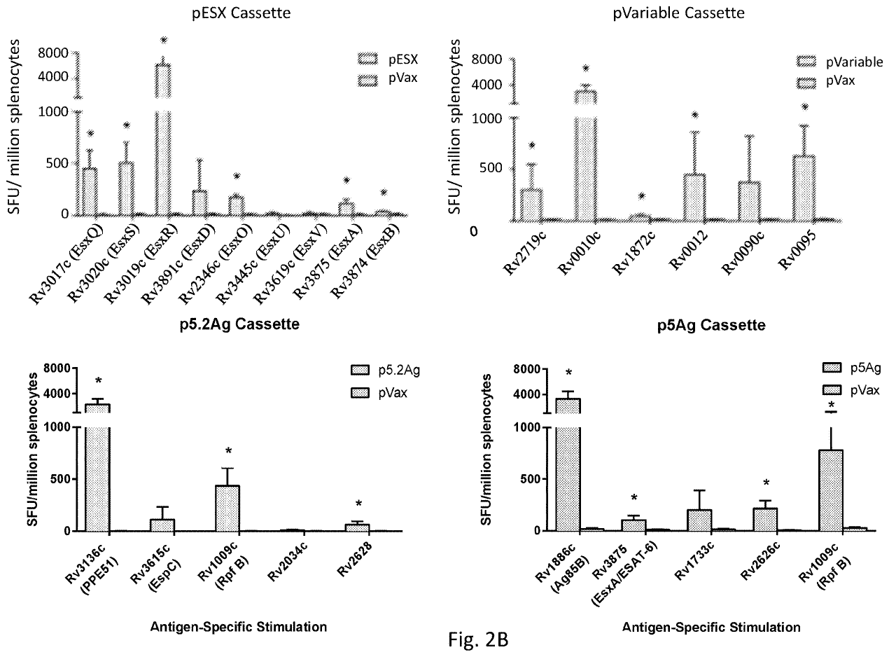

FIG. 2A and FIG. 2B show that multivalent vaccines induce broad and potent antigen-specific T cell responses. FIG. 2A shows the experimental layout. FIG. 2B shows results from harvested splenocytes stimulated with antigen-specific peptide pools were assessed for IFNγ production via ELISpot analysis.

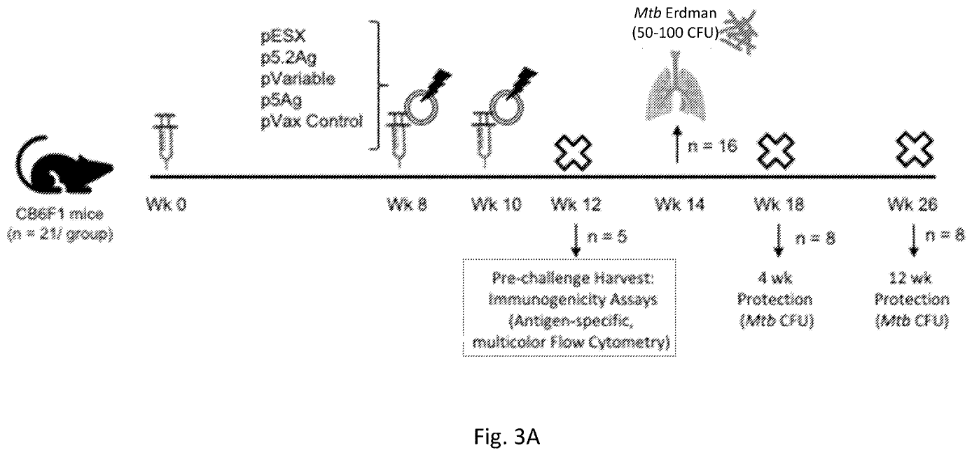

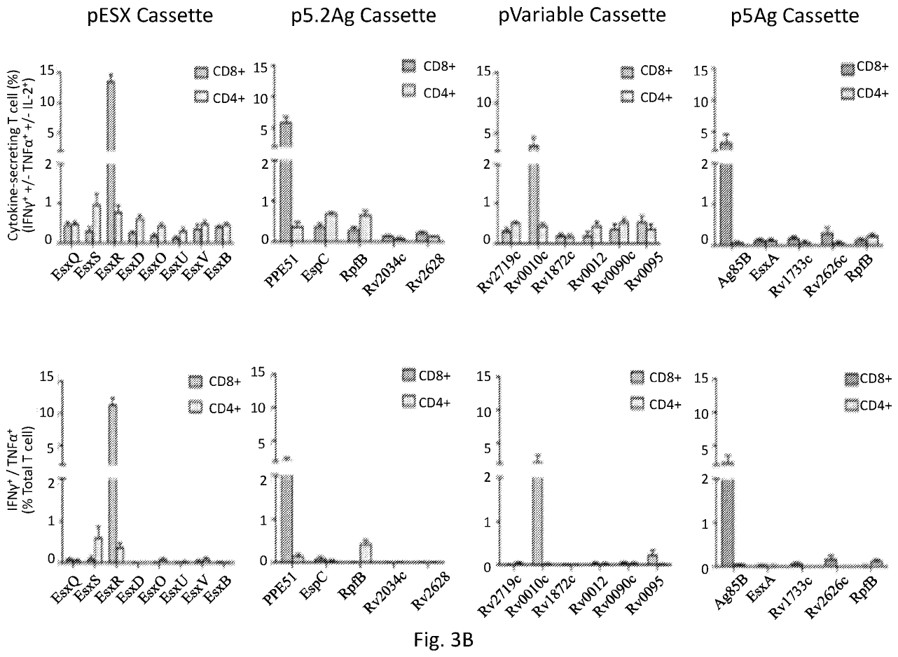

FIG. 3A and FIG. 3B shows humoral immune responses in response to multivalent vaccine administration. Multivalent vaccines induce both CD4+ and CD8+, polyfunctional cellular responses following a BCG prime. FIG. 3A depicts the experimental layout. FIG. 3B shows that harvested splenocytes stimulated with antigen-specific peptide pools were assessed for the total percentage of CD8+ and CD4+ T cells expressing INFγ, TNFα and/or IL-2 (top) or bifunctional for INFγ+/TNFα+ (bottom).

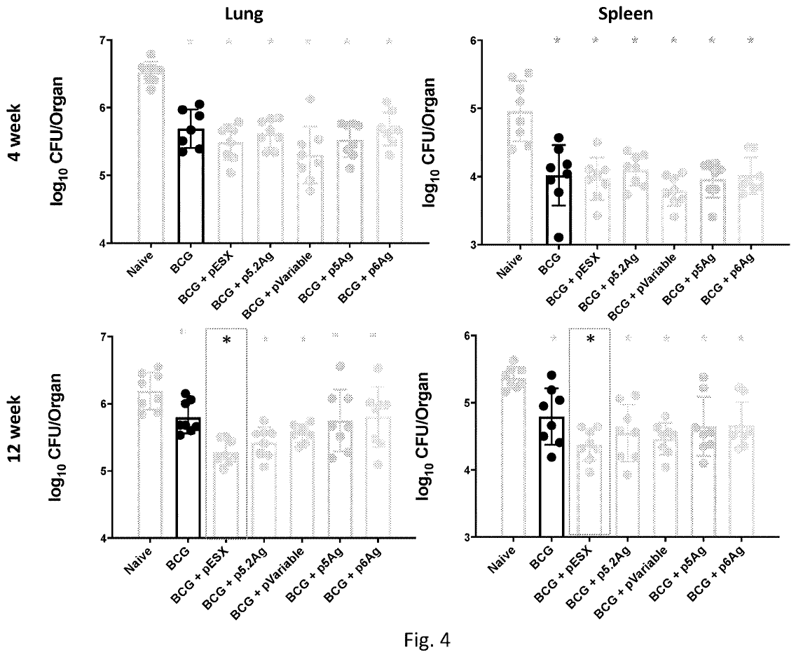

FIG. 4 shows vaccines prolong bacterial control following experimental M. tuberculosis challenge in BCG-primed animals. Mice were immunized as previously depicted and challenged with M. tuberculosis. Half of each group (n=8) was sacrificed 4 wks (top) or 12 wks (bottom) post challenge and bacterial burden was quantified in the lung (left) and spleen (right).

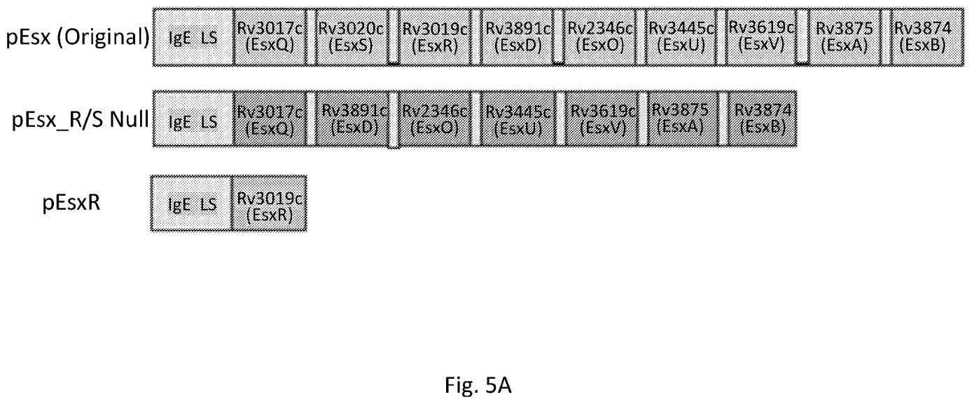

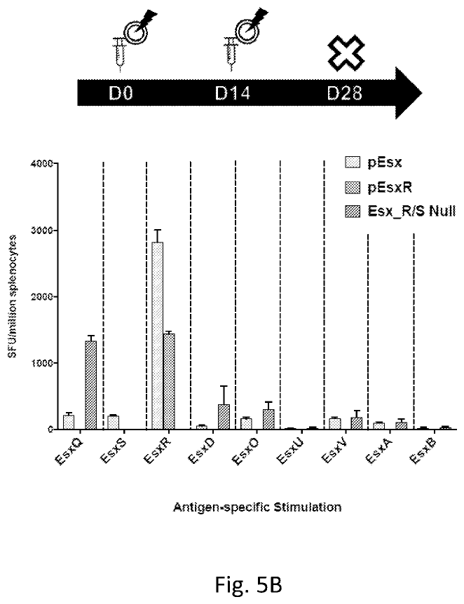

FIGS. 5A and 5B depict the design and immunogenicity of ESX-based vaccines. FIG. 5A shows the generation of two additional vaccine plasmids encoding select antigens derived from the parental pEsx construc. FIG. 5B shows that mice were immunized twice with the indicated vaccines and sacrificed two weeks later. Harvested splenocytes were stimulated with peptide pools representing the indicated antigens and assess for IFNγ production by ELISpot.

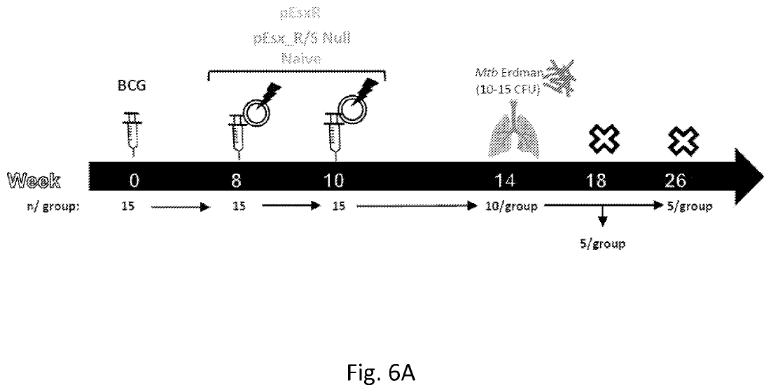

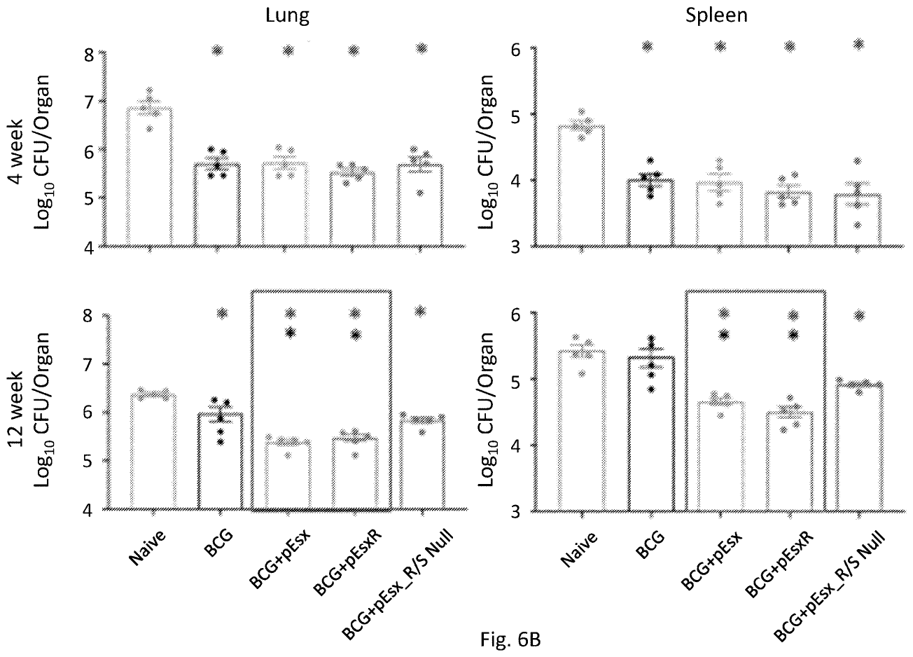

FIGS. 6A and 6B show that Mtb antigen EsxR is required and sufficient to confer pESC-mediated production. FIG. 6A shows the experimental design. Mice were immunized as previously depicted and challenged with M. tuberculosis. A subset of each group was sacrificed 4 wks (top) or 12 wks (bottom) post challenge and bacterial burden was quantified in the lung (left) and spleen (right).

Synthetic Multivalent TB vaccine is shown here to drive diverse and relevant immunity. This immune approach can be useful in immune therapy of TB patients or in Prime boost modalities or as a stand-alone approach for controlling TB infection.

| Multi-Valent | SEQ ID | | | |
|---|---|---|---|---|
| Plasmid | NO: | Type | Antigen | Sequence |
| pESX | SEQ ID | AA | Rv3017c | VSQSMYSYPAMTANVGDMAGYTGTTQSLGADIASERTAPSRAC |
| | NO 1 | | (EsxQ) | QGDLGMSHQDWQAQWNQAMEALARAYRRCRRALRQIGVLE |
| | | | | RPVGDSSDCGTIRVGSFRGRWLDPRHAGPATAADAGD |
| pESX | SEQ ID | DNA | Rv3017c | GTCTCACAGAGCATGTATTCTTACCCCGCAATGACCGCCAATG |
| | NO 2 | | (EsxQ) | TGGGCGACATGGCCGGCTACACAGGCACCACACAGTCCCTGG |
| | | | | GAGCAGATATCGCATCCGAGAGGACCGCACCCTCTCGCGCCT |
| | | | | GCCAGGGCGACCTGGGCATGTCTCACCAGGATTGGCAGGCCC |
| | | | | AGTGGAACCAGGCCATGGAGGCCCTGGCCAGAGCCTATAGG |
| | | | | AGATGCAGGCGCGCCCTGAGGCAGATCGGCGTGCTGGAGCG |
| | | | | CCCTGTGGGCGACAGCTCCGATTGTGGCACCATCAGAGTGGG |
| | | | | CTCTTTCAGGGGCAGATGGCTGGACCCACGGCACGCAGGACC |
| | | | | AGCAACAGCAGCAGACGCAGGCGAT |
| pESX | SEQ ID | AA | Rv3020c | SLLDAHIPQLIASHTAFAAKAGLMRHTIGQAEQQAMSAQAFHQ |
| | NO 3 | | (EsxS) | GESAAAFQGAHARFVAAAAKVNTLLDIAQANLGEAAGTYVAAD |
| | | | | AAAASSYTGF |
| pESX | SEQ ID | DNA | Rv3020c | AGCCTGCTGGATGCCCACATCCCACAGCTGATCGCATCCCACA |
| | NO 4 | | (EsxS) | CCGCCTTCGCCGCAAAGGCAGGCCTGATGCGCCACACAATCG |
| | | | | GACAGGCAGAGCAGCAGGCAATGTCTGCCCAGGCATTTCACC |
| | | | | AGGGAGAGAGCGCCGCAGCATTCCAGGGAGCACACGCAAGG |
| | | | | TTTGTGGCAGCAGCCGCCAAAGTGAATACCCTGCTGGACATC |
| | | | | GCACAGGCAAACCTGGGAGAGGCAGCAGGCACCTACGTGGC |
| | | | | CGCCGATGCCGCCGCCGCCTCCTCTTATACAGGCTTT |
| pESX | SEQ ID | AA | Rv3019c | SQIMYNYPAMMAHAGDMAGYAGTLQSLGADIASEQAVLSSA |
| | NO 5 | | (EsxR) | WQGDTGITYQGWQTQWNQALEDLVRAYQSMSGTHESNTMA |
| | | | | MLARDGAEAAKWGG |
| pESX | SEQ ID | DNA | Rv3019c | TCCCAGATCATGTACAACTATCCCGCCATGATGGCACACGCAG |
| | NO 6 | | (EsxR) | GCGACATGGCAGGATACGCAGGCACCCTGCAGTCTCTGGGCG |
| | | | | CCGATATCGCAAGCGAGCAGGCCGTGCTGTCTAGCGCCTGGC |
| | | | | AGGGCGACACCGGCATCACATACCAGGGCTGGCAGACACAGT |
| | | | | GGAATCAGGCCCTGGAGGATCTGGTGCGCGCCTATCAGTCTA |
| | | | | TGAGCGGCACCCACGAGAGCAACACAATGGCAATGCTGGCCA |
| | | | | GGGACGGAGCAGAGGCAGCAAAGTGGGGCGGC |
| pESX | SEQ ID | AA | Rv3891c | VADTIQVTPQMLRSTANDIQANMEQAMGIAKGYLANQENVM |
| | NO 7 | | (EsxD) | NPATWSGTGVVASHMTATEITNELNKVLTGGTRLAEGLVQAAA |
| | | | | LMEGHEADSQTAFQALFGASHGS |
| pESX | SEQ ID | DNA | Rv3891c | GTGGCCGACACCATCCAGGTGACACCTCAGATGCTGAGATCT |
| | NO 8 | | (EsxD) | ACCGCCAATGATATCCAGGCCAACATGGAGCAGGCCATGGGC |
| | | | | ATCGCCAAGGGCTACCTGGCCAACCAGGAGAATGTGATGAAC |
| | | | | CCAGCAACCTGGTCCGGAACAGGAGTGGTGGCATCTCACATG |
| | | | | ACCGCCACAGAGATCACAAATGAGCTGAACAAGGTGCTGACC |
| | | | | GGCGGCACAAGGCTGGCAGAGGGCCTGGTGCAGGCCGCCGC |
| | | | | CCTGATGGAGGGACACGAGGCAGACTCCCAGACCGCATTCCA |
| | | | | GGCCCTGTTTGGCGCCTCTCACGGCAGC |
| pESX | SEQ ID | AA | Rv2346c | TINYQFGDVDAHGAMIRAQAGLLEAEHQAIVRDVLAAGDFWG |
| | NO 9 | | (EsxO) | GAGSVACQEFITQLGRNFQVIYEQANAHGQKVQAAGNNMAQT |
| | | | | DSAVGSSWA |
| pESX | SEQ ID | DNA | Rv2346c | ACAATCAACTACCAGTTCGGCGACGTGGATGCACACGGAGCA |
| | NO 10 | | (EsxO) | ATGATCAGAGCACAGGCAGGCCTGCTGGAGGCAGAGCACCA |
| | | | | GGCAATCGTGCGGGACGTGCTGGCAGCAGGCGATTTTTGGGG |
| | | | | CGGCGCCGGCTCCGTGGCATGCCAGGAGTTCATCACCCAGCT |
| | | | | GGGCCGCAATTTTCAGGTCATCTACGAGCAGGCCAACGCACA |
| | | | | CGGACAGAAGGTGCAGGCAGCAGGCAACAATATGGCACAGA |
| | | | | CAGACAGCGCCGTGGGCTCCTCTTGGGCC |
| pESX | SEQ ID | AA | Rv3445c | VSTPNTLNADFDLMRSVAGITDARNEEIRAMLQAFIGRMSGVPP |
| | NO 11 | | (EsxU) | SVWGGLAAARFQDVVDRWNAESTRLYHVLHAIADTIRHNEAAL |
| | | | | REAGQIHARHIAAAGGDL |
| pESX | SEQ ID | DNA | Rv3445c | GTGTCTACCCCCAATACACTGAACGCCGACTTCGATCTGATGA |
| | NO 12 | | (EsxU) | GATCCGTGGCCGGCATCACCGATGCCAGGAATGAGGAGATCA |
| | | | | GAGCCATGCTGCAGGCCTTCATCGGAAGGATGTCTGGAGTGC |
| | | | | CCCCTAGCGTGTGGGGCGGCCTGGCAGCCGCCAGGTTTCAGG |
| | | | | ACGTGGTGGATAGATGGAATGCCGAGAGCACCCGGCTGTACC |
| | | | | ACGTGCTGCACGCCATCGCCGACACAATCAGGCACAACGAGG |
| | | | | CCGCCCTGAGGGAGGCCGGCCAGATCCACGCCAGACACATCG |
| | | | | CAGCAGCCGGCGGCGATCTG |
| pESX | SEQ ID | AA | Rv3619c | TINYQFGDVDAHGAMIRAQAGSLEAEHQAIISDVLTASDFWGG |
| | NO 13 | | (EsxV) | AGSAACQGFITQLGRNFQVIYEQANAHGQKVQAAGNNMAQT |
| | | | | DSAVGSSWA |
| pESX | SEQ ID | DNA | Rv3619c | ACCATCAACTATCAGTTTGGCGACGTGGACGCCCATGGAGCA |
| | NO 14 | | (EsxV) | ATGATCAGGGCACAGGCAGGCAGCCTGGAGGCCGAACACCA |
| | | | | GGCCATCATCAGCGACGTGCTGACCGCCTCTGACTTCTGGGGC |
| | | | | GGCGCCGGCTCCGCCGCATGTCAGGGCTTCATCACACAGCTG |
| | | | | GGCCGGAACTTCCAGGTCATCTACGAACAGGCTAATGCCCAT |
| | | | | GGCCAGAAAGTCCAGGCCGCAGGCAACAATATGGCCCAGACC |
| | | | | GACAGCGCCGTGGGCAGCTCCTGGGCC |
| pESX | SEQ ID | AA | Rv3875 | TEQQWNFAGIEAAASAIQGNVTSIHSLLDEGKQSLTKLAAAWGG |
| | NO 15 | | (EsxA) | SGSEAYQGVQQKWDATATELNNALQNLARTISEAGQAMASTE |
| | | | | GNVTGMFA |
| pESX | SEQ ID | DNA | Rv3875 | ACAGAGCAGCAGTGGAATTTCGCAGGAATCGAGGCAGCAGC |
| | NO 16 | | (EsxA) | ATCTGCCATCCAGGGCAACGTGACCTCCATCCACTCTCTGCTG |
| | | | | GACGAGGGCAAGCAGAGCCTGACAAAGCTGGCAGCAGCATG |
| | | | | GGGCGGCAGCGGCTCCGAGGCATATCAGGGAGTGCAGCAGA |
| | | | | AGTGGGATGCCACCGCCACAGAGCTGAACAATGCCCTGCAGA |
| | | | | ATCTGGCAAGGACCATCAGCGAGGCAGGACAGGCCATGGCCT |
| | | | | CCACCGAGGGCAACGTGACAGGCATGTTCGCC |
| pESX | SEQ ID | AA | Rv3874 | AEMKTDAATLAQEAGNFERISGDLKTQIDQVESTAGSLQGQWR |
| | NO 17 | | (EsxB) | GAAGTAAQAAVVRFQEAANKQKQELDEISTNIRQAGVQYSRAD |
| | | | | EEQQQALSSQMGF |
| pESX | SEQ ID | DNA | Rv3874 | GCCGAGATGAAGACCGACGCAGCCACACTGGCACAGGAGGC |
| | NO 18 | | (EsxB) | AGGCAACTTTGAGAGGATCTCCGGCGACCTGAAGACCCAGAT |
| | | | | CGATCAGGTGGAGAGCACAGCAGGCTCCCTGCAGGGCCAGT |
| | | | | GGAGGGGCGCCGCAGGAACCGCAGCACAGGCAGCAGTGGTG |
| | | | | AGGTTTCAGGAGGCAGCCAATAAGCAGAAGCAGGAGCTGGA |
| | | | | TGAGATCAGCACAAACATCAGGCAGGCCGGCGTCCAGTATTC |
| | | | | CAGGGCAGATGAAGAGCAGCAGCAGGCACTGTCAAGCCAGA |
| | | | | TGGGATTT |
| 5.2 | SEQ ID | AA | Rv3136c | DFALLPPEVNSARMYTGPGAGSLLAAAGGWDSLAAELATTAEAY |
| | NO 19 | | (PPE51) | GSVLSGLAALHWRGPAAESMAVTAAPYIGWLYTTAEKTQQTAI |
| | | | | QARAAALAFEQAYAMTLPPPVVAANRIQLLALIATNFFGQNTAAI |
| | | | | AATEAQYAEMWAQDAAAMYGYATASAAAALLTPFSPPRQTTN |
| | | | | PAGLTAQAAAVSQATDPLSLLIETVTQALQALTIPSFIPEDFTFLDA |
| | | | | IFAGYATVGVTQDVESFVAGTIGAESNLGLLNVGDENPAEVTPG |
| | | | | DFGIGELVSATSPGGGVSASGAGGAASVGNTVLASVGRANSIGQ |
| | | | | LSVPPSWAAPSTRPVSALSPAGLTTLPGTDVAEHGMPGVPGVPV |
| | | | | AAGRASGVLPRYGVRLTVMAHPPAAG |
| 5.2 | SEQ ID | DNA | Rv3136c | GATTTCGCTCTGCTGCCTCCTGAAGTCAACTCAGCAAGAATGT |
| | NO 20 | | (PPE51) | ACACCGGACCTGGAGCAGGCTCTCTGCTGGCAGCAGCCGGCG |
| | | | | GATGGGACAGCCTGGCCGCCGAGCTGGCCACCACAGCCGAG |
| | | | | GCCTATGGCTCCGTGCTGTCTGGCCTGGCCGCCCTGCACTGGA |
| | | | | GGGGACCTGCAGCCGAGAGCATGGCAGTGACCGCAGCACCA |
| | | | | TACATCGGATGGCTGTATACCACAGCCGAGAAGACACAGCAG |
| | | | | ACCGCAATCCAGGCCAGAGCCGCCGCCCTGGCCTTCGAGCAG |
| | | | | GCCTACGCCATGACACTGCCCCCTCCAGTGGTGGCCGCCAATA |
| | | | | GAATCCAGCTGCTGGCCCTGATCGCCACCAACTTCTTTGGCCA |
| | | | | GAATACAGCAGCAATCGCAGCAACCGAGGCACAGTATGCAGA |
| | | | | GATGTGGGCACAGGACGCAGCAGCAATGTACGGCTATGCCAC |
| | | | | AGCATCCGCCGCAGCCGCCCTGCTGACCCCCTTTTCTCCCCCTA |
| | | | | GACAGACCACAAACCCTGCAGGCCTGACAGCACAGGCAGCAG |
| | | | | CCGTGAGCCAGGCCACCGATCCACTGTCCCTGCTGATCGAGAC |
| | | | | AGTGACACAGGCCCTGCAGGCCCTGACAATCCCATCCTTCATC |
| | | | | CCCGAGGACTTCACCTTTCTGGATGCCATCTTTGCAGGATACG |
| | | | | CAACAGTGGGAGTGACCCAGGACGTGGAGAGCTTCGTGGCA |
| | | | | GGAACCATCGGAGCAGAGTCCAACCTGGGCCTGCTGAATGTG |
| | | | | GGCGACGAGAACCCAGCCGAGGTGACACCCGGCGATTTTGGC |
| | | | | ATCGGCGAGCTGGTGAGCGCCACCTCCCCCGGCGGCGGCGTG |
| | | | | AGCGCCTCCGGAGCCGGCGGAGCCGCAAGCGTGGGAAATAC |
| | | | | CGTGCTGGCAAGCGTGGGCAGAGCCAACTCCATCGGCCAGCT |
| | | | | GTCTGTGCCACCCAGCTGGGCAGCACCTTCCACACGGCCAGTG |
| | | | | TCTGCCCTGAGCCCAGCAGGCCTGACCACACTGCCTGGAACCG |
| | | | | ACGTGGCAGAGCACGGCATGCCAGGAGTGCCTGGAGTGCCA |
| | | | | GTGGCAGCCGGCAGAGCCAGCGGCGTGCTGCCACGCTACGG |
| | | | | CGTGAGGCTGACCGTGATGGCACACCCTCCAGCAGCAGGA |
| 5.2 | SEQ ID | AA | Rv3615c | TENLTVQPERLGVLASHHDNAAVDASSGVEAAAGLGESVAITHG |
| | NO 21 | | (EsxC) | PYCSQFNDTLNVYLTAHNALGSSLHTAGVDLAKSLRIAAKIYSEAD |
| | | | | EAWRKAIDGLFT |
| 5.2 | SEQ ID | DNA | Rv3615c | ACAGAGAATCTGACCGTGCAGCCAGAGCGGCTGGGCGTGCTG |
| | NO 22 | | (EsxC) | GCAAGCCACCACGACAACGCCGCCGTGGATGCCAGCTCCGGA |
| | | | | GTGGAGGCAGCAGCAGGCCTGGGAGAGTCTGTGGCCATCAC |
| | | | | ACACGGCCCATACTGCAGCCAGTTCAACGACACCCTGAACGTG |
| | | | | TACCTGACCGCCCACAATGCACTGGGCTCTAGCCTGCACACCG |
| | | | | CAGGAGTGGATCTGGCAAAGTCCCTGAGAATCGCCGCCAAGA |
| | | | | TCTACTCTGAGGCAGACGAGGCATGGAGGAAGGCAATCGATG |
| | | | | GCCTGTTCACC |
| 5.2 | SEQ ID | AA | Rv1009c | LRLVVGALLLVLAFAGGYAVAACKTVTLTVDGTAMRVTTMKSRV |
| | NO 23 | | (Rpf B) | IDIVEENGFSVDDRDDLYPAAGVQVHDADTIVLRRSRPLQISLDG |
| | | | | HDAKQVWTTASTVDEALAQLAMTDTAPAAASRASRVPLSGMA |
| | | | | LPVVSAKTVQLNDGGLVRTVHLPAPNVAGLLSAAGVPLLQSDHV |
| | | | | VPAATAPIVEGMQIQVTRNRIKKVTERLPLPPNARRVEDPEMNM |
| | | | | SREVVEDPGVPGTQDVTFAVAEVNGVETGRLPVANVVVTPAHE |
| | | | | AVVRVGTKPGTEVPPVIDGSIWDAIAGCEAGGNWAINTGNGYY |
| | | | | GGVQFDQGTWEANGGLRYAPRADLATREEQIAVAEVTRLRQG |
| | | | | WGAWPVCAARAGAR |
| 5.2 | SEQ ID | DNA | Rv1009c | CTGAGGCTGGTGGTGGGCGCCCTGCTGCTGGTGCTGGCCTTT |
| | NO 24 | | (Rpf B) | GCCGGCGGCTATGCAGTGGCAGCCTGTAAGACCGTGACACTG |
| | | | | ACCGTGGACGGAACCGCAATGAGGGTGACCACAATGAAGAG |
| | | | | CAGAGTGATCGATATCGTGGAGGAGAACGGCTTTTCCGTGGA |
| | | | | CGATAGGGACGATCTGTACCCTGCAGCAGGAGTGCAGGTGCA |
| | | | | CGACGCCGATACAATCGTGCTGAGGAGATCTAGGCCACTGCA |
| | | | | GATCAGCCTGGACGGCCACGATGCCAAGCAAGTGTGGACCAC |
| | | | | AGCATCCACCGTGGACGAGGCCCTGGCACAGCTGGCAATGAC |
| | | | | AGATACCGCACCTGCAGCAGCCTCCCGGGCCTCTCGCGTGCCA |
| | | | | CTGTCTGGAATGGCACTGCCAGTGGTGAGCGCCAAGACAGTG |
| | | | | CAGCTGAATGACGGCGGCCTGGTGAGGACCGTGCACCTGCCT |
| | | | | GCACCAAACGTGGCAGGCCTGCTGAGCGCCGCAGGAGTGCCA |
| | | | | CTGCTGCAGTCCGATCACGTGGTGCCTGCAGCAACCGCACCAA |
| | | | | TCGTGGAGGGAATGCAGATCCAGGTGACACGGAATCGCATCA |
| | | | | AGAAGGTGACCGAGCGCCTGCCACTGCCCCCTAATGCAAGGC |
| | | | | GCGTGGAGGACCCTGAGATGAACATGTCTAGGGAGGTGGTG |
| | | | | GAGGACCCCGGAGTGCCTGGCACACAGGATGTGACCTTCGCC |
| | | | | GTGGCCGAAGTGAATGGAGTGGAGACAGGCCGCCTGCCTGT |
| | | | | GGCAAACGTGGTGGTGACCCCAGCACACGAGGCAGTGGTGA |
| | | | | GGGTGGGAACAAAGCCAGGAACCGAGGTGCCACCCGTGATC |
| | | | | GACGGCTCCATCTGGGATGCAATCGCAGGATGCGAGGCCGGC |
| | | | | GGAAATTGGGCCATCAACACAGGCAATGGCTACTATGGCGGC |
| | | | | GTGCAGTTTGACCAGGGAACCTGGGAGGCAAACGGCGGCCT |
| | | | | GAGATACGCCCCTCGGGCCGATCTGGCAACACGCGAGGAGCA |
| | | | | GATCGCAGTGGCAGAGGTGACCAGGCTGAGACAGGGATGGG |
| | | | | GCGCCTGGCCCGTGTGCGCAGCCAGAGCCGGCGCCAGG |
| 5.2 | SEQ ID | AA | Rv2034c | VSTYRSPDRAWQALADGTRRAIVERLAHGPLAVGELARDLPVSR |
| | NO 25 | | | PAVSQHLKVLKTARLVCDRPAGTRRVYQLDPTGLAALRTDLDRF |
| | | | | WTRALTGYAQLIDSEGDDT |
| 5.2 | SEQ ID | DNA | Rv2034c | GTGAGCACATATCGCAGCCCCGACAGGGCATGGCAGGCCCTG |
| | NO 26 | | | GCAGATGGAACCAGGAGAGCCATCGTGGAGAGACTGGCACA |
| | | | | CGGACCTCTGGCAGTGGGAGAGCTGGCCAGAGATCTGCCCGT |
| | | | | GTCCCGGCCTGCCGTGTCTCAGCACCTGAAGGTGCTGAAGAC |
| | | | | AGCAAGGCTGGTGTGCGACAGGCCAGCAGGAACCAGGCGCG |
| | | | | TGTACCAGCTGGACCCCACAGGCCTGGCCGCCCTGCGCACCG |
| | | | | ACCTGGATAGATTCTGGACACGGGCCCTGACCGGATATGCAC |
| | | | | AGCTGATCGACTCCGAGGGCGACGATACA |
| 5.2 | SEQ ID | AA | Rv2628c | STQRPRHSGIRAVGPYAWAGRCGRIGRWGVHQEAMMNLAIW |
| | NO 27 | | | HPRKVQSATIYQVTDRSHDGRTARVPGDEITSTVSGWLSELGTQ |
| | | | | SPLADELARAVRIGDWPAAYAIGEHLSVEIAVAV |
| 5.2 | SEQ ID | DNA | Rv2628c | TCTACCCAGAGGCCAAGACACTCTGGCATCAGGGCAGTGGGA |
| | NO 28 | | | CCATACGCATGGGCAGGCAGGTGTGGAAGGATCGGCCGGTG |
| | | | | GGGCGTGCACCAGGAGGCAATGATGAACCTGGCCATCTGGCA |
| | | | | CCCTCGCAAGGTGCAGTCCGCCACAATCTATCAGGTGACCGAC |
| | | | | AGGAGCCACGATGGAAGGACAGCCAGGGTGCCAGGCGACGA |
| | | | | GATCACAAGCACCGTGTCCGGATGGCTGTCCGAGCTGGGAAC |
| | | | | CCAGTCTCCTCTGGCAGATGAGCTGGCCAGAGCCGTGCGGAT |
| | | | | CGGCGATTGGCCCGCCGCCTACGCTATTGGGGAACACCTGTCC |
| | | | | GTGGAGATTGCTGTCGCAGTG |
| Var | SEQ ID | AA | Rv2719c | TPVRPPHTPDPLNLRGPLDGPRWRRAEPAQSRRPGRSRPGGAP |
| | NO 29 | | | LRYHRTGVGMSRTGHGSRPVPPATTVGLALLAAAITLWLGLVAQ |
| | | | | FGQMITGGSADGSADSTGRVPDRLAVVRVETGESLYDVAVRVA |
| | | | | PNAPTRQVADRIRELNGLQTPALAVGQTLIAPVG |
| Var | SEQ ID | DNA | Rv2719c | ACTCCTGTCAGGCCCCCACATACTCCCGATCCCCTGAATCTGA |
| | NO 30 | | | GAGGCCCACTGGACGGCCCCCGGTGGAGGAGAGCAGAGCCT |
| | | | | GCACAGTCCCGGCGCCCAGGCAGATCTCGGCCTGGCGGCGCC |
| | | | | CCACTGCGCTACCACAGGACAGGAGTGGGAATGAGCAGGAC |
| | | | | CGGACACGGCTCCAGGCCTGTGCCCCCTGCCACCACAGTGGG |
| | | | | CCTGGCCCTGCTGGCAGCAGCCATCACCCTGTGGCTGGGCCTG |
| | | | | GTGGCACAGTTCGGACAGATGATCACCGGCGGCTCCGCCGAC |
| | | | | GGCTCCGCCGATTCTACAGGAAGGGTGCCAGACAGGCTGGCA |
| | | | | GTGGTGCGGGTCGAGACAGGAGAGTCTCTGTATGATGTGGCC |
| | | | | GTGCGCGTGGCACCAAACGCACCAACAAGGCAGGTGGCAGA |
| | | | | CCGCATCAGGGAGCTGAATGGCCTGCAGACCCCTGCCCTGGC |
| | | | | AGTGGGACAGACACTGATCGCACCAGTGGGA |
| Var | SEQ ID | AA | Rv0010c | QQTAWAPRTSGIAGCGAGGVVMAIASVTLVTDTPGRVLTGVAA |
| | NO 31 | | | LGLILFASATWRARPRLAITPDGLAIRGWFRTQLLRHSNIKIIRIDEF |
| | | | | RRYGRLVRLLEIETVSGGLLILSRWDLGTDPVEVLDALTAAGYAGR |
| | | | | GQR |
| Var | SEQ ID | DNA | Rv0010c | CAGCAGACAGCCTGGGCCCCACGGACCAGCGGAATCGCAGG |
| | NO 32 | | | ATGCGGAGCCGGCGGCGTGGTCATGGCCATCGCCAGCGTGAC |
| | | | | CCTGGTGACCGATACACCAGGACGCGTGCTGACAGGAGTGGC |
| | | | | CGCCCTGGGCCTGATCCTGTTTGCATCCGCCACCTGGAGGGCC |
| | | | | AGGCCCAGACTGGCAATCACACCTGACGGCCTGGCAATCAGG |
| | | | | GGATGGTTCAGGACCCAGCTGCTGAGACACTCCAATATCAAG |
| | | | | ATCATCCGGATCGATGAGTTTCGGCGCTACGGCAGACTGGTG |
| | | | | CGGCTGCTGGAGATCGAGACAGTGTCTGGCGGCCTGCTGATC |
| | | | | CTGAGCAGATGGGACCTGGGAACCGATCCCGTGGAGGTGCTG |
| | | | | GACGCACTGACAGCAGCAGGATATGCAGGAAGAGGACAGAG |
| | | | | G |
| Var | SEQ ID | AA | Rv1872c | AVNRRVPRVRDLAPLLQFNRPQFDTSKRRLGAALTIQDLRRIAKR |
| | NO 33 | | | RTPRAAFDYADGGAEDELSIARARQGFRDIEFHPTILRDVTTVCA |
| | | | | GWNVLGQPTVLPFGIAPTGFTRLMHTEGEIAGARAAAAAGIPFS |
| | | | | LSTLATCAIEDLVIAVPQGRKWFQLYMWRDRDRSMALVRRVAA |
| | | | | AGFDTMLVTVDVPVAGARLRDVRNGMSIPPALTLRTVLDAMGH |
| | | | | PRWWFDLLTTEPLAFASLDRWPGTVGEYLNTVFDPSLTFDDLA |
| | | | | WIKSQWPGKLVVKGIQTLDDARAVVDRGVDGIVLSNHGGRQLD |
| | | | | RAPVPFHLLPHVARELGKHTEILVDTGIMSGADIVAAIALGARCTL |
| | | | | IGRAYLYGLMAGGEAGVNRAIEILQTGVIRTMRLLGVTCLEELSPR |
| | | | | HVTQLRRLGPIGAPT |
| Var | SEQ ID | DNA | Rv1872c | GCCGTGAACAGGAGAGTGCCCCGGGTGCGCGACCTGGCCCCT |
| | NO 34 | | | CTGCTGCAGTTCAATAGGCCACAGTTTGATACCAGCAAGCGGC |
| | | | | GCCTGGGAGCCGCCCTGACAATCCAGGACCTGAGGAGAATCG |
| | | | | CAAAGAGGAGGACCCCTAGAGCCGCCTTCGACTACGCAGATG |
| | | | | GCGGAGCCGAGGATGAGCTGTCCATCGCCAGGGCCAGACAG |
| | | | | GGCTTCAGAGACATCGAGTTTCACCCTACAATCCTGCGGGATG |
| | | | | TGACCACAGTGTGCGCAGGATGGAACGTGCTGGGACAGCCAA |
| | | | | CCGTGCTGCCTTTCGGCATCGCACCAACAGGCTTTACCAGACT |
| | | | | GATGCACACAGAGGGCGAGATCGCAGGAGCCAGGGCCGCAG |
| | | | | CAGCAGCAGGCATCCCCTTTTCTCTGAGCACACTGGCCACCTG |
| | | | | TGCCATCGAGGACCTGGTCATCGCCGTGCCTCAGGGCAGGAA |
| | | | | GTGGTTCCAGCTGTACATGTGGCGGGACCGCGATAGGAGCAT |
| | | | | GGCACTGGTGAGGAGAGTGGCAGCAGCAGGCTTTGACACAA |
| | | | | TGCTGGTGACCGTGGATGTGCCAGTGGCAGGAGCAAGACTGA |
| | | | | GGGATGTGCGGAACGGCATGTCCATCCCACCCGCCCTGACAC |
| | | | | TGAGGACCGTGCTGGACGCAATGGGACACCCAAGGTGGTGG |
| | | | | TTCGATCTGCTGACCACAGAGCCCCTGGCCTTTGCCTCTCTGG |
| | | | | ACAGGTGGCCTGGAACAGTGGGAGAGTATCTGAATACCGTGT |
| | | | | TCGATCCCAGCCTGACATTTGACGATCTGGCCTGGATCAAGTC |
| | | | | CCAGTGGCCTGGCAAGCTGGTGGTGAAGGGCATCCAGACCCT |
| | | | | GGACGATGCCAGAGCCGTGGTGGACCGGGGCGTGGATGGAA |
| | | | | TCGTGCTGTCTAACCACGGCGGCAGACAGCTGGACAGGGCAC |
| | | | | CAGTGCCTTTCCACCTGCTGCCACACGTGGCCCGGGAGCTGG |
| | | | | GCAAGCACACAGAGATCCTGGTGGACACCGGCATCATGAGCG |
| | | | | GCGCCGATATCGTGGCAGCAATCGCCCTGGGAGCAAGGTGCA |
| | | | | CCCTGATCGGCAGGGCCTACCTGTATGGCCTGATGGCCGGCG |
| | | | | GCGAGGCAGGAGTGAACAGGGCCATCGAGATCCTGCAGACA |
| | | | | GGCGTGATCCGCACCATGAGGCTGCTGGGCGTGACCTGTCTG |
| | | | | GAGGAGCTGTCTCCCCGCCACGTGACACAGCTGCGGCGCCTG |
| | | | | GGACCAATCGGAGCACCCACC |
| Var | SEQ ID | AA | Rv0012 | RLTHPTPCPENGETMIDRRRSAWRFSVPLVCLLAGLLLAATHGVS |
| | NO 35 | | | GGTEIRRSDAPRLVDLVRRAQASVNRLATEREALTTRIDSVHGRS |
| | | | | VDTALAAMQRRSAKLAGVAAMNPVHGPGLVVTLQDAQRDAN |
| | | | | GRFPRDASPDDLVVHQQDIEAVLNALWNAGAEAIQMQDQRIIA |
| | | | | MSIARCVGNTLLLNGRTYSPPYTIAAIGDAAAMQAALAAAPLVTL |
| | | | | YKQYVVRFGLGYCEEVHPDLQIVGYADPVRMHFAQPAGPLDY |
| Var | SEQ ID | DNA | Rv0012 | AGGCTGACACACCCAACCCCCTGCCCTGAGAATGGCGAGACA |
| | NO 36 | | | ATGATCGACCGGCGCAGGTCCGCCTGGAGATTCTCTGTGCCCC |
| | | | | TGGTGTGCCTGCTGGCAGGCCTGCTGCTGGCAGCAACCCACG |
| | | | | GCGTGAGCGGCGGCACAGAGATCAGACGGTCCGACGCCCCTA |
| | | | | GACTGGTGGATCTGGTGCGCAGGGCACAGGCAAGCGTGAAC |
| | | | | AGGCTGGCAACCGAGAGGGAGGCCCTGACCACACGCATCGA |
| | | | | CAGCGTGCACGGCAGGTCCGTGGATACAGCCCTGGCAGCAAT |
| | | | | GCAGAGACGGTCCGCCAAGCTGGCAGGAGTGGCAGCAATGA |
| | | | | ACCCTGTGCACGGACCAGGCCTGGTGGTGACCCTGCAGGACG |
| | | | | CACAGAGAGATGCCAATGGCAGATTTCCCCGGGACGCATCCC |
| | | | | CTGACGATCTGGTGGTGCACCAGCAGGATATCGAGGCCGTGC |
| | | | | TGAACGCACTGTGGAATGCAGGAGCAGAGGCAATCCAGATGC |
| | | | | AGGACCAGCGCATCATCGCCATGTCCATCGCCAGATGCGTGG |
| | | | | GCAACACCCTGCTGCTGAATGGCCGGACATACTCTCCTCCATA |
| | | | | TACCATCGCCGCCATCGGCGATGCAGCAGCAATGCAGGCCGC |
| | | | | CCTGGCAGCAGCCCCTCTGGTGACCCTGTACAAGCAGTATGTG |
| | | | | GTGCGGTTCGGCCTGGGCTACTGTGAGGAGGTGCACCCAGAC |
| | | | | CTGCAGATCGTGGGCTATGCCGATCCCGTGCGCATGCACTTTG |
| | | | | CACAGCCTGCAGGACCACTGGACTAC |
| Var | SEQ ID | AA | Rv0090c | VAESSLNPSLVSRISAFLRPDWTRTVRARRFAAAGLVMLAGVAAL |
| | NO 37 | | | RSNPEDDRSEVVVAAHDLRPGTALTPGDVRLEKRSATTLPDGSQ |
| | | | | ADLDAVVGSTLASPTRRGEVLTDVRLLGSRLAESTAGPDARIVPL |
| | | | | HLADSALVDLVRVGDVVDVLAAPVTDSPAALRLLATDAIVVLVSA |
| | | | | QQKAQAADSDRVVLVALPARLANTVAGAALGQTVTLTLH |
| Var | SEQ ID | DNA | Rv0090c | GTGGCAGAGAGCTCCCTGAACCCAAGCCTGGTGTCCAGAATC |
| | NO 38 | | | TCTGCCTTCCTGCGGCCTGATTGGACCCGCACAGTGAGGGCAA |
| | | | | GACGGTTTGCAGCAGCAGGCCTGGTCATGCTGGCAGGAGTGG |
| | | | | CCGCCCTGAGATCCAATCCAGAGGACGATCGGTCTGAGGTGG |
| | | | | TGGTGGCAGCACACGACCTGAGGCCAGGAACAGCCCTGACCC |
| | | | | CAGGCGATGTGCGCCTGGAGAAGAGGTCTGCCACCACACTGC |
| | | | | CTGACGGCAGCCAGGCAGACCTGGATGCAGTGGTGGGCAGC |
| | | | | ACACTGGCATCCCCAACCAGGAGGGGCGAGGTGCTGACCGAC |
| | | | | GTGAGACTGCTGGGCTCTCGGCTGGCAGAGAGCACAGCAGG |
| | | | | ACCTGATGCAAGAATCGTGCCACTGCACCTGGCAGACAGCGC |
| | | | | CCTGGTGGATCTGGTGCGGGTGGGCGACGTGGTGGATGTGCT |
| | | | | GGCAGCACCAGTGACCGACTCCCCAGCCGCCCTGAGACTGCT |
| | | | | GGCCACAGATGCCATCGTGGTGCTGGTGTCCGCCCAGCAGAA |
| | | | | GGCACAGGCAGCCGACTCTGATAGAGTGGTGCTGGTGGCCCT |
| | | | | GCCCGCCCGGCTGGCCAATACCGTGGCAGGAGCCGCCCTGGG |
| | | | | ACAGACCGTGACACTGACCCTGCAC |
| Var | SEQ ID | AA | Rv0095 | AVGPLRVSAGVIRLRPVRMRDGVHWSRIRLADRAHLEPWEPSA |
| | NO 39 | | | DGEWTVRHTVAAWPAVCSGLRSEARNGRMLPYVIELDGQFCG |
| | | | | QLTIGNVTHGALRSAWIGYWVPSAATGGGVATGALALGLDHCF |
| | | | | GPVMLHRVEATVRPENAASRAVLAKVGFREEGLLRRYLEVDRA |
| | | | | WRDHLLMAITVEEVYGSVASTLVRAGHASWP |
| Var | SEQ ID | DNA | Rv0095 | GCCGTGGGACCACTGAGGGTGTCTGCCGGCGTGATCAGACTG |
| | NO 40 | | | CGGCCCGTGCGCATGAGGGACGGAGTGCACTGGTCTAGAATC |
| | | | | CGGCTGGCAGATAGAGCACACCTGGAGCCATGGGAGCCTAGC |
| | | | | GCCGACGGAGAGTGGACAGTGCGCCACACCGTGGCAGCATG |
| | | | | GCCAGCCGTGTGCTCTGGCCTGAGGAGCGAGGCAAGAAACG |
| | | | | GAAGGATGCTGCCCTACGTGATCGAGCTGGATGGCCAGTTCT |
| | | | | GTGGCCAGCTGACAATCGGCAATGTGACCCACGGCGCCCTGA |
| | | | | GGAGCGCCTGGATCGGCTATTGGGTGCCTTCCGCCGCAACCG |
| | | | | GCGGCGGCGTGGCAACCGGCGCCCTGGCCCTGGGCCTGGACC |
| | | | | ACTGTTTCGGACCTGTGATGCTGCACAGGGTGGAGGCAACCG |
| | | | | TGAGGCCAGAGAACGCAGCCAGCCGCGCCGTGCTGGCCAAA |
| | | | | GTGGGCTTTAGGGAGGAGGGCCTGCTGCGCAGGTATCTGGA |
| | | | | GGTGGACCGCGCCTGGAGGGATCACCTGCTGATGGCCATCAC |
| | | | | CGTGGAGGAGGTCTACGGGAGCGTCGCAAGCACACTGGTCA |
| | | | | GAGCAGGACACGCAAGCTGGCCT |
| 5 | SEQ ID | AA | Rv1886c | TDVSRKIRAWGRRLMIGTAAAVVLPGLVGLAGGAATAGAFSRP |
| | NO 41 | | (Ag85B) | GLPVEYLQVPSPSMGRDIKVQFQSGGNNSPAVYLLDGLRAQDD |
| | | | | YNGWDINTPAFEWYYQSGLSIVMPVGGQSSFYSDWYSPACGKA |
| | | | | GCQTYKWETFLTSELPQWLSANRAVKPTGSAAIGLSMAGSSAMI |
| | | | | LAAYHPQQFIYAGSLSALLDPSQGMGPSLIGLAMGDAGGYKAAD |
| | | | | MWGPSSDPAWERNDPTQQIPKLVANNTRLWVYCGNGTPNEL |
| | | | | GGANIPAEFLENFVRSSNLKFQDAYNAAGGHNAVFNFPPNGTH |
| | | | | SWEYWGAQLNAMKGDLQSSLGAG |
| 5 | SEQ ID | DNA | Rv1886c | ACCGATGTCAGCAGGAAGATTAGGGCTTGGGGCAGGAGACT |
| | NO 42 | | (Ag85B) | GATGATCGGCACAGCCGCCGCCGTGGTGCTGCCAGGCCTGGT |
| | | | | GGGCCTGGCCGGCGGAGCCGCAACCGCAGGCGCCTTCAGCA |
| | | | | GGCCAGGCCTGCCAGTGGAGTACCTGCAGGTGCCTTCCCCATC |
| | | | | TATGGGCAGAGATATCAAGGTGCAGTTTCAGTCTGGCGGCAA |
| | | | | CAATAGCCCTGCCGTGTACCTGCTGGATGGCCTGAGGGCCCA |
| | | | | GGACGATTATAATGGCTGGGACATCAACACCCCAGCCTTCGA |
| | | | | GTGGTACTATCAGAGCGGCCTGTCCATCGTGATGCCAGTGGG |
| | | | | CGGCCAGAGCTCCTTTTACAGCGACTGGTATTCCCCAGCATGC |
| | | | | GGCAAGGCAGGATGTCAGACATATAAGTGGGAGACATTCCTG |
| | | | | ACCTCTGAGCTGCCACAGTGGCTGAGCGCCAATAGAGCCGTG |
| | | | | AAGCCAACCGGCTCCGCCGCAATCGGCCTGTCTATGGCAGGC |
| | | | | TCTAGCGCCATGATCCTGGCAGCCTACCACCCTCAGCAGTTTA |
| | | | | TCTATGCAGGCAGCCTGTCCGCCCTGCTGGACCCCAGCCAGG |
| | | | | GAATGGGACCTTCCCTGATCGGCCTGGCAATGGGCGACGCCG |
| | | | | GCGGATACAAGGCAGCAGATATGTGGGGACCCTCCTCTGACC |
| | | | | CTGCATGGGAGCGGAACGATCCAACCCAGCAGATCCCCAAGC |
| | | | | TGGTGGCCAACAATACACGCCTGTGGGTGTATTGCGGAAACG |
| | | | | GAACCCCAAATGAGCTGGGCGGCGCCAATATCCCTGCCGAGT |
| | | | | TCCTGGAGAATTTTGTGCGGAGCTCCAACCTGAAGTTCCAGGA |
| | | | | TGCATACAACGCAGCCGGCGGACACAACGCCGTGTTCAATTTT |
| | | | | CCCCCTAACGGCACACACAGCTGGGAGTATTGGGGCGCCCAG |
| | | | | CTGAACGCAATGAAGGGCGACCTGCAGTCTAGCCTGGGAGCA |
| | | | | GGA |
| 5 | SEQ ID | DNA | Rv3875 | ACCGAGCAGCAGTGGAATTTTGCAGGAATCGAGGCAGCAGCA |
| | NO 43 | | (EsxA) | TCCGCCATCCAGGGCAACGTGACATCTATCCACAGCCTGCTGG |
| | | | | ATGAGGGCAAGCAGTCCCTGACCAAGCTGGCAGCAGCATGG |
| | | | | GGCGGCTCTGGCAGCGAGGCATACCAGGGAGTGCAGCAGAA |
| | | | | GTGGGACGCAACCGCCACAGAGCTGAACAATGCCCTGCAGAA |
| | | | | TCTGGCAAGGACCATCAGCGAGGCAGGACAGGCCATGGCCTC |
| | | | | CACAGAGGGCAACGTGACCGGCATGTTCGCC |
| 5 | SEQ ID | AA | Rv1733c | IATTRDREGATMITFRLRLPCRTILRVFSRNPLVRGTDRLEAVVML |
| | NO 44 | | | LAVTVSLLTIPFAAAAGTAVQDSRSHVYAHQAQTRHPATATVID |
| | | | | HEGVIDSNTTATSAPPRTKITVPARWVVNGIERSGEVNAKPGTKS |
| | | | | GDRVGIWVDSAGQLVDEPAPPARAIADAALAALGLWLSVAAVA |
| | | | | GALLALTRAILIRVRNASWQHDIDSLFCTQR |
| 5 | SEQ ID | DNA | Rv1733c | ATCGCAACCACACGGGACCGCGAGGGAGCAACAATGATCACC |
| | NO 45 | | | TTCAGGCTGAGACTGCCCTGTCGGACAATCCTGCGCGTGTTTT |
| | | | | CTAGGAATCCTCTGGTGAGAGGCACCGATAGGCTGGAGGCAG |
| | | | | TGGTCATGCTGCTGGCAGTGACAGTGAGCCTGCTGACCATCCC |
| | | | | CTTTGCCGCAGCAGCAGGAACAGCAGTGCAGGACAGCAGGA |
| | | | | GCCACGTGTACGCACACCAGGCACAGACCAGGCACCCTGCAA |
| | | | | CCGCAACAGTGATCGATCACGAGGGCGTGATCGACTCTAACA |
| | | | | CCACAGCCACAAGCGCCCCACCCCGCACAAAGATCACCGTGCC |
| | | | | CGCCAGATGGGTGGTGAATGGCATCGAGAGATCCGGCGAGG |
| | | | | TGAACGCCAAGCCTGGAACCAAGTCTGGCGACCGGGTGGGA |
| | | | | ATCTGGGTGGATAGCGCCGGACAGCTGGTGGACGAGCCCGCC |
| | | | | CCTCCAGCCAGGGCCATCGCAGACGCCGCCCTGGCCGCCCTG |
| | | | | GGCCTGTGGCTGTCCGTGGCCGCCGTGGCCGGCGCCCTGCTG |
| | | | | GCCCTGACCCGGGCCATCCTGATCCGGGTGCGCAATGCCTCCT |
| | | | | GGCAGCACGATATCGACTCTCTGTTCTGCACACAGAGG |
| 5 | SEQ ID | AA | Rv2626c | TTARDIMNAGVTCVGEHETLTAAAQYMREHDIGALPICGDDDRL |
| | NO 46 | | | HGMLTDRDIVIKGLAAGLDPNTATAGELARDSIYYVDANASIQE |
| | | | | MLNVMEEHQVRRVPVISEHRLVGIVTEADIARHLPEHAIVQFVK |
| | | | | AICSPMALAS |
| 5 | SEQ ID | DNA | Rv2626c | ACCACAGCCCGCGATATCATGAACGCAGGAGTGACCTGCGTG |
| | NO 47 | | | GGAGAGCACGAGACACTGACAGCAGCAGCACAGTACATGAG |
| | | | | GGAGCACGACATCGGCGCCCTGCCAATCTGCGGCGACGATGA |
| | | | | CAGGCTGCACGGCATGCTGACAGATAGAGACATCGTGATCAA |
| | | | | GGGCCTGGCAGCAGGCCTGGACCCCAATACCGCAACAGCAGG |
| | | | | AGAGCTGGCCCGCGATTCCATCTACTATGTGGACGCCAATGCC |
| | | | | TCTATCCAGGAGATGCTGAACGTGATGGAGGAGCACCAGGTG |
| | | | | CGGAGGGTGCCCGTGATCAGCGAGCACAGGCTGGTGGGCAT |
| | | | | CGTGACCGAGGCCGACATCGCCAGACACCTGCCTGAGCACGC |
| | | | | CATCGTGCAGTTCGTGAAGGCCATCTGCTCCCCAATGGCCCTG |
| | | | | GCCTCT |
| 5 | SEQ ID | DNA | Rv1009c | CTGAGGCTGGTGGTGGGCGCCCTGCTGCTGGTGCTGGCCTTT |
| | NO 48 | | (Rpf B) | GCCGGCGGCTACGCAGTGGCAGCCTGTAAGACCGTGACACTG |
| | | | | ACCGTGGATGGCACCGCCATGAGAGTGACCACAATGAAGAGC |
| | | | | AGAGTGATCGACATCGTGGAGGAGAACGGCTTCTCTGTGGAT |
| | | | | GACAGGGATGACCTGTATCCAGCAGCAGGAGTGCAGGTGCAC |
| | | | | GATGCAGACACAATCGTGCTGCGGCGCAGCAGACCCCTGCAG |
| | | | | ATCTCCCTGGATGGCCACGACGCCAAGCAAGTGTGGACCACA |
| | | | | GCAAGCACCGTGGATGAGGCCCTGGCACAGCTGGCAATGACA |
| | | | | GACACCGCACCTGCAGCAGCCTCCAGGGCCTCTAGAGTGCCA |
| | | | | CTGTCTGGAATGGCACTGCCAGTGGTGAGCGCCAAGACAGTG |
| | | | | CAGCTGAATGATGGCGGCCTGGTGAGAACCGTGCACCTGCCA |
| | | | | GCCCCTAACGTGGCAGGCCTGCTGAGCGCCGCAGGAGTGCCA |
| | | | | CTGCTGCAGTCCGACCACGTGGTGCCTGCAGCAACCGCACCA |
| | | | | ATCGTGGAGGGAATGCAGATCCAGGTGACACGGAACCGCATC |
| | | | | AAGAAGGTGACCGAGAGGCTGCCTCTGCCCCCTAATGCCAGG |
| | | | | AGAGTGGAGGACCCAGAGATGAACATGTCTAGAGAGGTGGT |
| | | | | GGAGGACCCCGGAGTGCCAGGAACCCAGGACGTGACCTTCGC |
| | | | | CGTGGCCGAAGTGAATGGAGTGGAGACAGGCCGGCTGCCTG |
| | | | | TGGCAAACGTGGTGGTGACCCCAGCACACGAGGCAGTGGTGC |
| | | | | GCGTGGGAACAAAGCCAGGAACCGAGGTGCCACCCGTGATC |
| | | | | GATGGCTCCATCTGGGACGCAATCGCAGGATGTGAGGCCGGC |
| | | | | GGAAATTGGGCCATCAACACAGGCAATGGCTACTATGGCGGC |
| | | | | GTGCAGTTTGATCAGGGAACCTGGGAGGCAAACGGCGGCCT |
| | | | | GCGCTACGCCCCTAGGGCCGACCTGGCAACCAGGGAGGAGC |
| | | | | AGATCGCAGTGGCAGAGGTGACAAGGCTGAGACAGGGGTGG |
| | | | | GGAGCATGGCCCGTGTGCGCTGCAAGAGCTGGAGCAAGA |
| R/S Null | SEQ ID | DNA | Rv3017c | GTGAGCCAGTCCATGTACAGCTATCCTGCCATGACCGCCAATG |
| | NO 49 | | (EsxQ) | TGGGCGACATGGCCGGCTACACAGGCACCACACAGTCCCTGG |
| | | | | GAGCAGATATCGCATCTGAGAGGACCGCACCAAGCCGCGCCT |
| | | | | GCCAGGGCGACCTGGGAATGTCCCACCAGGATTGGCAGGCCC |
| | | | | AGTGGAACCAGGCAATGGAGGCCCTGGCCAGGGCCTATAGG |
| | | | | AGATGCAGGCGCGCCCTGAGGCAGATCGGCGTGCTGGAGCG |
| | | | | CCCAGTGGGCGACAGCTCCGATTGTGGCACCATCCGCGTGGG |
| | | | | CAGCTTTAGGGGCAGATGGCTGGACCCAAGGCACGCAGGACC |
| | | | | AGCAACAGCAGCAGACGCAGGCGAT |
| R/S Null | SEQ ID | DNA | Rv3891c | GTGGCAGACACCATCCAGGTGACACCCCAGATGCTGCGCTCC |
| | NO 50 | | (EsxD) | ACCGCCAATGATATCCAGGCCAACATGGAGCAGGCCATGGGC |
| | | | | ATCGCCAAGGGCTACCTGGCCAACCAGGAGAATGTGATGAAC |
| | | | | CCTGCAACCTGGAGCGGAACAGGAGTGGTGGCATCCCACATG |
| | | | | ACCGCCACAGAGATCACAAATGAGCTGAACAAGGTGCTGACC |
| | | | | GGCGGCACAAGGCTGGCAGAGGGACTGGTGCAGGCAGCCGC |
| | | | | CCTGATGGAGGGCCACGAGGCCGATTCTCAGACCGCATTCCA |
| | | | | GGCCCTGTTTGGAGCAAGCCACGGCTCC |
| R/S Null | SEQ ID | DNA | Rv2346c | ACAATCAACTACCAGTTCGGCGACGTGGATGCACACGGAGCC |
| | NO 51 | | (EsxO) | ATGATCAGGGCACAGGCAGGACTGCTGGAGGCAGAGCACCA |
| | | | | GGCAATCGTGAGGGACGTGCTGGCAGCAGGCGATTTTTGGG |
| | | | | GAGGAGCAGGCTCCGTGGCATGCCAGGAGTTCATCACCCAGC |
| | | | | TGGGCCGCAATTTTCAGGTCATCTACGAGCAGGCCAACGCAC |
| | | | | ACGGACAGAAGGTGCAGGCAGCAGGCAACAATATGGCCCAG |
| | | | | ACAGACTCTGCCGTGGGCTCTAGCTGGGCC |
| R/S Null | SEQ ID | DNA | Rv3445c | GTGAGCACCCCCAATACACTGAACGCCGACTTCGATCTGATGA |
| | NO 52 | | (EsxU) | GAAGCGTGGCCGGCATCACCGATGCCAGGAATGAGGAGATC |
| | | | | AGAGCCATGCTGCAGGCCTTCATCGGAAGGATGAGCGGAGTG |
| | | | | CCACCTTCCGTGTGGGGAGGACTGGCCGCCGCCAGGTTTCAG |
| | | | | GACGTGGTGGATAGATGGAATGCCGAGTCCACCCGGCTGTAC |
| | | | | CACGTGCTGCACGCCATCGCCGACACAATCCGCCACAACGAG |
| | | | | GCCGCCCTGAGGGAGGCCGGCCAGATCCACGCAAGGCACATC |
| | | | | GCAGCAGCAGGAGGCGATCTG |
| R/S Null | SEQ ID | DNA | Rv3619c | ACCATCAACTATCAGTTTGGCGACGTGGACGCACACGGAGCA |
| | NO 53 | | (EsxV) | ATGATTAGAGCACAGGCAGGCTCCCTGGAGGCCGAACACCAG |
| | | | | GCCATCATCTCCGACGTGCTGACCGCCTCTGACTTCTGGGGAG |
| | | | | GAGCAGGCAGCGCCGCATGTCAGGGCTTCATCACACAGCTGG |
| | | | | GCAGGAACTTCCAGGTCATCTACGAACAGGCTAATGCCCATG |
| | | | | GCCAGAAAGTCCAGGCCGCAGGCAACAATATGGCACAGACCG |
| | | | | ACTCTGCCGTGGGCTCCTCTTGGGCC |
| R/S Null | SEQ ID | DNA | Rv3875 | ACAGAGCAGCAGTGGAATTTCGCAGGAATCGAGGCAGCAGC |
| | NO 54 | | (EsxA) | ATCCGCCATCCAGGGCAACGTGACCTCTATCCACAGCCTGCTG |
| | | | | GACGAGGGCAAGCAGTCTCTGACAAAGCTGGCAGCAGCATG |
| | | | | GGGAGGCTCCGGCTCTGAGGCATATCAGGGCGTGCAGCAGA |
| | | | | AGTGGGATGCCACCGCCACAGAGCTGAACAATGCCCTGCAGA |
| | | | | ATCTGGCCAGAACCATCTCTGAGGCAGGACAGGCAATGGCAA |
| | | | | GCACCGAGGGCAACGTGACAGGCATGTTCGCC |
| R/S Null | SEQ ID | DNA | Rv3874 | GCCGAGATGAAGACCGACGCAGCCACACTGGCACAGGAGGC |
| | NO 55 | | (EsxB) | AGGCAACTTTGAGCGGATCTCTGGCGACCTGAAGACCCAGAT |
| | | | | CGATCAGGTGGAGTCCACAGCCGGCTCTCTGCAGGGCCAGTG |
| | | | | GAGAGGAGCAGCAGGAACCGCAGCACAGGCAGCAGTGGTGC |
| | | | | GGTTCCAGGAGGCCGCCAATAAGCAGAAGCAGGAGCTGGAC |
| | | | | GAGATCTCCACAAACATCAGACAGGCCGGCGTGCAGTATTCTC |
| | | | | GGGCCGATGAGGAGCAGCAGCAGGCCCTGAGCTCCCAGATG |
| | | | | GGCTTT |
| R | SEQ ID | DNA | Rv3019c | TCCCAGATCATGTACAACTATCCCGCCATGATGGCACACGCAG |
| | NO 56 | | (EsxR) | GCGACATGGCAGGATACGCAGGCACCCTGCAGTCCCTGGGAG |
| | | | | CAGATATCGCCTCTGAGCAGGCCGTGCTGTCTAGCGCCTGGCA |
| | | | | GGGCGACACCGGCATCACATACCAGGGCTGGCAGACACAGTG |
| | | | | GAACCAGGCCCTGGAGGATCTGGTGAGGGCCTATCAGTCTAT |
| | | | | GAGCGGCACCCACGAGTCTAATACAATGGCCATGCTGGCCAG |
| | | | | AGACGGAGCAGAGGCAGCAAAGTGGGGAGGA |

| | |
|---|---|
| (SEQ ID NO: 57). | amino acid sequence for pEsx |
| (SEQ ID NO: 58). | nucleic acid sequence for pEsx |
| (SEQ ID NO: 59). | amino acid sequence for p5.2Ag |
| (SEQ ID NO: 60). | nucleic acid sequence for p5.2Ag |
| (SEQ ID NO: 61). | amino acid sequence for pVariable |
| (SEQ ID NO: 62). | nucleic acid sequence for pVariable |
| (SEQ ID NO: 63). | amino acid sequence for p5Ag |
| (SEQ ID NO: 64). | nucleic acid sequence for p5Ag |
| (SEQ ID NO: 65). | amino acid sequence for pEsx_R/S Null |
| (SEQ ID NO: 66). | nucleic acid sequence for pEsx_R/S Null |
| (SEQ ID NO: 67) | amino acid sequence for pEsxR |
| (SEQ ID NO: 68). | nucleic acid sequence for pEsxR |
| (SEQ ID NO: 69). | amino acid sequence for IgE leader sequence |

pEsx: AA sequence
(SEQ ID NO: 57)
MDWTWILFLVAAATRVHSVSQSMYSYPAMTANVGDMAGYTGTTQSLGADIASERTA
PSRACQGDLGMSHQDWQAQWNQAMEALARAYRRCRRALRQIGVLERPVGDSSDCGTI
RVGSFRGRWLDPRHAGPATAADAGDRGRKRRSSLLDAHIPQLIASHTAFAAKAGLMR
HTIGQAEQQAMSAQAFHQGESAAAFQGAHARFVAAAAKVNTLLDIAQANLGEAAGTY
VAADAAAASSYTGFRGRKRRSSQIMYNYPAMMAHAGDMAGYAGTLQSLGADIASEQ
AVLSSAWQGDTGITYQGWQTQWNQALEDLVRAYQSMSGTHESNTMAMLARDGAEAA
KWGGRGRKRRSVADTIQVTPQMLRSTANDIQANMEQAMGIAKGYLANQENVMNPAT
WSGTGVVASHMTATEITNELNKVLTGGTRLAEGLVQAAALMEGHEADSQTAFQALFG
ASHGSRGRKRRSTINYQFGDVDAHGAMIRAQAGLLEAEHQAIVRDVLAAGDFWGGAG
SVACQEFITQLGRNFQVIYEQANAHGQKVQAAGNNMAQTDSAVGSSWARGRKRRSVS
TPNTLNADFDLMRSVAGITDARNEEIRAMLQAFIGRMSGVPPSVWGGLAAARFQDVVD
RWNAESTRLYHVLHALADTIRHNEAALREAGQIHARHIAAAGGDLRGRKRRSTINYQF
GDVDAHGAMIRAQAGSLEAEHQAIISDVLTASDFWGGAGSAACQGFITQLGRNFQVIYE
QANAHGQKVQAAGNNMAQTDSAVGSSWARGRKRRSTEQQWNFAGIEAAASAIQGNV
TSIHSLLDEGKQSLTKLAAAWGGSGSEAYQGVQQKWDATATELNNALQNLARTISEAG
QAMASTEGNVTGMFARGRKRRSAEMKTDAATLAQEAGNFERISGDLKTQIDQVESTA
GSLQGQWRGAAGTAAQAAVVRFQEAANKQKQELDEISTNIRQAGVQYSRADEEQQQA
LSSQMGF*
pEsx: nt sequenc (IgE leader sequence is underlined)
(SEQ ID NO: 58)
ATGGACTGGACTTGGATTCTGTTCCTGGTCGCCGCCGCAACTAGGGTGCATAGCGTC
TCACAGAGCATGTATTCTTACCCCGCAATGACCGCCAATGTGGGCGACATGGCCGGC
TACACAGGCACCACACAGTCCCTGGGAGCAGATATCGCATCCGAGAGGACCGCACC
CTCTCGCGCCTGCCAGGGCGACCTGGGCATGTCTCACCAGGATTGGCAGGCCCAGTG
GAACCAGGCCATGGAGGCCCTGGCCAGAGCCTATAGGAGATGCAGGCGCGCCCTGA
GGCAGATCGGCGTGCTGGAGCGCCCTGTGGGCGACAGCTCCGATTGTGGCACCATC
AGAGTGGGCTCTTTCAGGGGCAGATGGCTGGACCCACGGCACGCAGGACCAGCAAC
AGCAGCAGACGCAGGCGATCGGGGAAGGAAGAGGAGATCTAGCCTGCTGGATGCC
CACATCCCACAGCTGATCGCATCCCACACCGCCTTCGCCGCAAAGGCAGGCCTGATG
CGCCACACAATCGGACAGGCAGAGCAGCAGGCAATGTCTGCCCAGGCATTTCACCA
GGGAGAGAGCGCCGCAGCATTCCAGGGAGCACACGCAAGGTTTGTGGCAGCAGCCG
CCAAAGTGAATACCCTGCTGGACATCGCACAGGCAAACCTGGGAGAGGCAGCAGGC
ACCTACGTGGCCGCCGATGCCGCCGCCGCCTCCTCTTATACAGGCTTTAGGGGCAGA
AAGCGGCGCAGCTCCCAGATCATGTACAACTATCCCGCCATGATGGCACACGCAGG
CGACATGGCAGGATACGCAGGCACCCTGCAGTCTCTGGGCGCCGATATCGCAAGCG
AGCAGGCCGTGCTGTCTAGCGCCTGGCAGGGCGACACCGGCATCACATACCAGGGC
TGGCAGACACAGTGGAATCAGGCCCTGGAGGATCTGGTGCGCGCCTATCAGTCTAT
GAGCGGCACCCACGAGAGCAACACAATGGCAATGCTGGCCAGGGACGGAGCAGAG
GCAGCAAAGTGGGGCGGCCGGGGCCGCAAGAGGAGATCCGTGGCCGACACCATCC
AGGTGACACCTCAGATGCTGAGATCTACCGCCAATGATATCCAGGCCAACATGGAG
CAGGCCATGGGCATCGCCAAGGGCTACCTGGCCAACCAGGAGAATGTGATGAACCC
AGCAACCTGGTCCGGAACAGGAGTGGTGGCATCTCACATGACCGCCACAGAGATCA
CAAATGAGCTGAACAAGGTGCTGACCGGCGGCACAAGGCTGGCAGAGGGCCTGGTG
CAGGCCGCCGCCCTGATGGAGGGACACGAGGCAGACTCCCAGACCGCATTCCAGGC
CCTGTTTGGCGCCTCTCACGGCAGCAGGGGCAGGAAACGGCGCTCTACAATCAACT
ACCAGTTCGGCGACGTGGATGCACACGGAGCAATGATCAGAGCACAGGCAGGCCTG
CTGGAGGCAGAGCACCAGGCAATCGTGCGGGACGTGCTGGCAGCAGGCGATTTTTG
GGGCGGCGCCGGCTCCGTGGCATGCCAGGAGTTCATCACCCAGCTGGGCCGCAATT
TTCAGGTCATCTACGAGCAGGCCAACGCACACGGACAGAAGGTGCAGGCAGCAGGC
AACAATATGGCACAGACAGACAGCGCCGTGGGCTCCTCTTGGGCCAGGGGCAGGAA
GAGGAGATCCGTGTCTACCCCCAATACACTGAACGCCGACTTCGATCTGATGAGATC
CGTGGCCGGCATCACCGATGCCAGGAATGAGGAGATCAGAGCCATGCTGCAGGCCT
TCATCGGAAGGATGTCTGGAGTGCCCCCTAGCGTGTGGGGCGGCCTGGCAGCCGCC
AGGTTTCAGGACGTGGTGGATAGATGGAATGCCGAGAGCACCCGGCTGTACCACGT
GCTGCACGCCATCGCCGACACAATCAGGCACAACGAGGCCGCCCTGAGGGAGGCCG
GCCAGATCCACGCCAGACACATCGCAGCAGCCGGCGGCGATCTGAGGGGAAGAAA
GCGGCGCTCTACCATCAACTATCAGTTTGGCGACGTGGACGCCCATGGAGCAATGAT
CAGGGCACAGGCAGGCAGCCTGGAGGCCGAACACCAGGCCATCATCAGCGACGTGC
TGACCGCCTCTGACTTCTGGGGCGGCGCCGGCTCCGCCGCATGTCAGGGCTTCATCA
CACAGCTGGGCCGGAACTTCCAGGTCATCTACGAACAGGCTAATGCCCATGGCCAG
AAAGTCCAGGCCGCAGGCAACAATATGGCCCAGACCGACAGCGCCGTGGGCAGCTC
CTGGGCCAGGGGCAGAAAAAGGAGATCCACAGAGCAGCAGTGGAATTTCGCAGGA
ATCGAGGCAGCAGCATCTGCCATCCAGGGCAACGTGACCTCCATCCACTCTCTGCTG
GACGAGGGCAAGCAGAGCCTGACAAAGCTGGCAGCAGCATGGGGCGGCAGCGGCT
CCGAGGCATATCAGGGAGTGCAGCAGAAGTGGGATGCCACCGCCACAGAGCTGAAC
AATGCCCTGCAGAATCTGGCAAGGACCATCAGCGAGGCAGGACAGGCCATGGCCTC
CACCGAGGGCAACGTGACAGGCATGTTCGCCAGGGGCAGGAAGCGGCGCAGCGCC
GAGATGAAGACCGACGCAGCCACACTGGCACAGGAGGCAGGCAACTTTGAGAGGA
TCTCCGGCGACCTGAAGACCCAGATCGATCAGGTGGAGAGCACAGCAGGCTCCCTG
CAGGGCCAGTGGAGGGGCGCCGCAGGAACCGCAGCACAGGCAGCAGTGGTGAGGT
TTCAGGAGGCAGCCAATAAGCAGAAGCAGGAGCTGGATGAGATCAGCACAAACATC
AGGCAGGCCGGCGTCCAGTATTCCAGGGCAGATGAAGAGCAGCAGCAGGCACTGTC
AAGCCAGATGGGATTTTGATAA
P5.2Ag: AA sequence
(SEQ ID NO: 59)
MDWTWILFLVAAATRVHSDFALLPPEVNSARMYTGPGAGSLLAAAGGWDSLAAELA
TTAEAYGSVLSGLAALHWRGPAAESMAVTAAPYIGWLYTTAEKTQQTAIQARAAALAF
EQAYAMTLPPPVVAANRIQLLALIATNFFGQNTAAIAATEAQYAEMWAQDAAAMYGY
ATASAAAALLTPFSPPRQTTNPAGLTAQAAAVSQATDPLSLLIETVTQALQALTIPSFIPE
DFTFLDAIFAGYATVGVTQDVESFVAGTIGAESNLGLLNVGDENPAEVTPGDFGIGELVS
ATSPGGGVSASGAGGAASVGNTVLASVGRANSIGQLSVPPSWAAPSTRPVSALSPAGLT
TLPGTDVAEHGMPGVPGVPVAAGRASGVLPRYGVRLTVMAHPPAAGRGRKRRSTENL
TVQPERLGVLASHHDNAAVDASSGVEAAAGLGESVAITHGPYCSQFNDTLNVYLTAHN
ALGSSLHTAGVDLAKSLRIAAKIYSEADEAWRKAIDGLFTRGRKRRSLRLVVGALLLVL
AFAGGYAVAACKTVTLTVDGTAMRVTTMKSRVIDIVEENGFSVDDRDDLYPAAGVQV
HDADTIVLRRSRPLQISLDGHDAKQVWTTASTVDEALAQLAMTDTAPAAASRASRVPL
SGMALPVVSAKTVQLNDGGLVRTVHLPAPNVAGLLSAAGVPLLQSDHVVPAATAPIVE
GMQIQVTRNRIKKVTERLPLPPNARRVEDPEMNMSREVVEDPGVPGTQDVTFAVAEVN
GVETGRLPVANVVVTPAHEAVVRVGTKPGTEVPPVIDGSIWDAIAGCEAGGNWAINTG
NGYYGGVQFDQGTWEANGGLRYAPRADLATREEQIAVAEVTRLRQGWGAWPVCAAR
AGARRGRKRRSVSTYRSPDRAWQALADGTRRAIVERLAHGPLAVGELARDLPVSRPA
VSQHLKVLKTARLVCDRPAGTRRVYQLDPTGLAALRTDLDRFWTRALTGYAQLIDSEG
DDTRGRKRRSSTQRPRHSGIRAVGPYAWAGRCGRIGRWGVHQEAMMNLAIWHPRKV
QSATIYQVTDRSHDGRTARVPGDEITSTVSGWLSELGTQSPLADELARAVRIGDWPAAY
AIGEHLSVEIAVAV*
P5.2Ag: nt sequence (IgE leader sequence is underlined)
(SEQ ID NO: 60)
ATGGATTGGACATGGATTCTGTTCCTGGTCGCCGCCGCCACACGGGTGCATTCCGAT
TTCGCTCTGCTGCCTCCTGAAGTCAACTCAGCAAGAATGTACACCGGACCTGGAGCA
GGCTCTCTGCTGGCAGCAGCCGGCGGATGGGACAGCCTGGCCGCCGAGCTGGCCAC
CACAGCCGAGGCCTATGGCTCCGTGCTGTCTGGCCTGGCCGCCCTGCACTGGAGGGG
ACCTGCAGCCGAGAGCATGGCAGTGACCGCAGCACCATACATCGGATGGCTGTATA
CCACAGCCGAGAAGACACAGCAGACCGCAATCCAGGCCAGAGCCGCCGCCCTGGCC
TTCGAGCAGGCCTACGCCATGACACTGCCCCCTCCAGTGGTGGCCGCCAATAGAATC
CAGCTGCTGGCCCTGATCGCCACCAACTTCTTTGGCCAGAATACAGCAGCAATCGCA
GCAACCGAGGCACAGTATGCAGAGATGTGGGCACAGGACGCAGCAGCAATGTACG
GCTATGCCACAGCATCCGCCGCAGCCGCCCTGCTGACCCCCTTTTCTCCCCCTAGAC
AGACCACAAACCCTGCAGGCCTGACAGCACAGGCAGCAGCCGTGAGCCAGGCCACC
GATCCACTGTCCCTGCTGATCGAGACAGTGACACAGGCCCTGCAGGCCCTGACAATC
CCATCCTTCATCCCCGAGGACTTCACCTTTCTGGATGCCATCTTTGCAGGATACGCA
ACAGTGGGAGTGACCCAGGACGTGGAGAGCTTCGTGGCAGGAACCATCGGAGCAGA
GTCCAACCTGGGCCTGCTGAATGTGGGCGACGAGAACCCAGCCGAGGTGACACCCG
GCGATTTTGGCATCGGCGAGCTGGTGAGCGCCACCTCCCCCGGCGGCGGCGTGAGC
GCCTCCGGAGCCGGCGGAGCCGCAAGCGTGGGAAATACCGTGCTGGCAAGCGTGGG
CAGAGCCAACTCCATCGGCCAGCTGTCTGTGCCACCCAGCTGGGCAGCACCTTCCAC
ACGGCCAGTGTCTGCCCTGAGCCCAGCAGGCCTGACCACACTGCCTGGAACCGACG
TGGCAGAGCACGGCATGCCAGGAGTGCCTGGAGTGCCAGTGGCAGCCGGCAGAGCC
AGCGGCGTGCTGCCACGCTACGGCGTGAGGCTGACCGTGATGGCACACCCTCCAGC
AGCAGGAAGGGGACGCAAGAGGAGATCTACAGAGAATCTGACCGTGCAGCCAGAG
CGGCTGGGCGTGCTGGCAAGCCACCACGACAACGCCGCCGTGGATGCCAGCTCCGG
AGTGGAGGCAGCAGCAGGCCTGGGAGAGTCTGTGGCCATCACACACGGCCCATACT
GCAGCCAGTTCAACGACACCCTGAACGTGTACCTGACCGCCCACAATGCACTGGGC
TCTAGCCTGCACACCGCAGGAGTGGATCTGGCAAAGTCCCTGAGAATCGCCGCCAA
GATCTACTCTGAGGCAGACGAGGCATGGAGGAAGGCAATCGATGGCCTGTTCACCA
GGGGCAGAAAGCGGCGCAGCCTGAGGCTGGTGGTGGGCGCCCTGCTGCTGGTGCTG
GCCTTTGCCGGCGGCTATGCAGTGGCAGCCTGTAAGACCGTGACACTGACCGTGGA
CGGAACCGCAATGAGGGTGACCACAATGAAGAGCAGAGTGATCGATATCGTGGAGG
AGAACGGCTTTTCCGTGGACGATAGGGACGATCTGTACCCTGCAGCAGGAGTGCAG
GTGCACGACGCCGATACAATCGTGCTGAGGAGATCTAGGCCACTGCAGATCAGCCT
GGACGGCCACGATGCCAAGCAAGTGTGGACCACAGCATCCACCGTGGACGAGGCCC
TGGCACAGCTGGCAATGACAGATACCGCACCTGCAGCAGCCTCCCGGGCCTCTCGC
GTGCCACTGTCTGGAATGGCACTGCCAGTGGTGAGCGCCAAGACAGTGCAGCTGAA
TGACGGCGGCCTGGTGAGGACCGTGCACCTGCCTGCACCAAACGTGGCAGGCCTGC
TGAGCGCCGCAGGAGTGCCACTGCTGCAGTCCGATCACGTGGTGCCTGCAGCAACC
GCACCAATCGTGGAGGGAATGCAGATCCAGGTGACACGGAATCGCATCAAGAAGGT
GACCGAGCGCCTGCCACTGCCCCCTAATGCAAGGCGCGTGGAGGACCCTGAGATGA
ACATGTCTAGGGAGGTGGTGGAGGACCCCGGAGTGCCTGGCACACAGGATGTGACC
TTCGCCGTGGCCGAAGTGAATGGAGTGGAGACAGGCCGCCTGCCTGTGGCAAACGT
GGTGGTGACCCCAGCACACGAGGCAGTGGTGAGGGTGGGAACAAAGCCAGGAACC
GAGGTGCCACCCGTGATCGACGGCTCCATCTGGGATGCAATCGCAGGATGCGAGGC
CGGCGGAAATTGGGCCATCAACACAGGCAATGGCTACTATGGCGGCGTGCAGTTTG
ACCAGGGAACCTGGGAGGCAAACGGCGGCCTGAGATACGCCCCTCGGGCCGATCTG
GCAACACGCGAGGAGCAGATCGCAGTGGCAGAGGTGACCAGGCTGAGACAGGGAT
GGGGCGCCTGGCCCGTGTGCGCAGCCAGAGCCGGCGCCAGGAGAGGCCGGAAGCG
GCGCTCTGTGAGCACATATCGCAGCCCCGACAGGGCATGGCAGGCCCTGGCAGATG
GAACCAGGAGAGCCATCGTGGAGAGACTGGCACACGGACCTCTGGCAGTGGGAGA
GCTGGCCAGAGATCTGCCCGTGTCCCGGCCTGCCGTGTCTCAGCACCTGAAGGTGCT
GAAGACAGCAAGGCTGGTGTGCGACAGGCCAGCAGGAACCAGGCGCGTGTACCAG
CTGGACCCCACAGGCCTGGCCGCCCTGCGCACCGACCTGGATAGATTCTGGACACG
GGCCCTGACCGGATATGCACAGCTGATCGACTCCGAGGGCGACGATACACGGGGCC
GCAAGAGGAGATCCTCTACCCAGAGGCCAAGACACTCTGGCATCAGGGCAGTGGGA
CCATACGCATGGGCAGGCAGGTGTGGAAGGATCGGCCGGTGGGGCGTGCACCAGGA
GGCAATGATGAACCTGGCCATCTGGCACCCTCGCAAGGTGCAGTCCGCCACAATCT
ATCAGGTGACCGACAGGAGCCACGATGGAAGGACAGCCAGGGTGCCAGGCGACGA
GATCACAAGCACCGTGTCCGGATGGCTGTCCGAGCTGGGAACCCAGTCTCCTCTGGC
AGATGAGCTGGCCAGAGCCGTGCGGATCGGCGATTGGCCCGCCGCCTACGCTATTG
GGGAACACCTGTCCGTGGAGATTGCTGTCGCAGTGTGATAA
pVariable: AA sequence
(SEQ ID NO: 61)
MDWTWILFLVAAATRVHSTPVRPPHTPDPLNLRGPLDGPRWRRAEPAQSRRPGRSRPG
GAPLRYHRTGVGMSRTGHGSRPVPPATTVGLALLAAAITLWLGLVAQFGQMITGGSAD
GSADSTGRVPDRLAVVRVETGESLYDVAVRVAPNAPTRQVADRIRELNGLQTPALAVG
QTLIAPVGRGRKRRSQQTAWAPRTSGIAGCGAGGVVMAIASVTLVTDTPGRVLTGVAA
LGLILFASATWRARPRLAITPDGLAIRGWFRTQLLRHSNIKIIRIDEFRRYGRLVRLLEIET
VSGGLLILSRWDLGTDPVEVLDALTAAGYAGRGQRRGRKRRSAVNRRVPRVRDLAPL
LQFNRPQFDTSKRRLGAALTIQDLRRIAKRRTPRAAFDYADGGAEDELSIARARQGFRDI
EFHPTILRDVTTVCAGWNVLGQPTVLPFGIAPTGFTRLMHTEGEIAGARAAAAAGIPFSL
STLATCAIEDLVIAVPQGRKWFQLYMWRDRDRSMALVRRVAAAGFDTMLVTVDVPVA
GARLRDVRNGMSIPPALTLRTVLDAMGHPRWWFDLLTTEPLAFASLDRWPGTVGEYLN
TVFDPSLTFDDLAWIKSQWPGKLVVKGIQTLDDARAVVDRGVDGIVLSNHGGRQLDRA
PVPFHLLPHVARELGKHTEILVDTGIMSGADIVAAIALGARCTLIGRAYLYGLMAGGEA
GVNRAIEILQTGVIRTMRLLGVTCLEELSPRHVTQLRRLGPIGAPTRGRKRRSRLTHPTP
CPENGETMIDRRRSAWRFSVPLVCLLAGLLLAATHGVSGGTEIRRSDAPRLVDLVRRAQ
ASVNRLATEREALTTRIDSVHGRSVDTALAAMQRRSAKLAGVAAMNPVHGPGLVVTL
QDAQRDANGRFPRDASPDDLVVHQQDIEAVLNALWNAGAEAIQMQDQRIIAMSIARCV
GNTLLLNGRTYSPPYTIAAIGDAAAMQAALAAAPLVTLYKQYVVRFGLGYCEEVHPDL
QIVGYADPVRMHFAQPAGPLDYRGRKRRSVAESSLNPSLVSRISAFLRPDWTRTVRAR
RFAAAGLVMLAGVAALRSNPEDDRSEVVVAAHDLRPGTALTPGDVRLEKRSATTLPDG
SQADLDAVVGSTLASPTRRGEVLTDVRLLGSRLAESTAGPDARIVPLHLADSALVDLVR
VGDVVDVLAAPVTDSPAALRLLATDAIVVLVSAQQKAQAADSDRVVLVALPARLANT
VAGAALGQTVTLTLHRGRKRRSAVGPLRVSAGVIRLRPVRMRDGVHWSRIRLADRAH
LEPWEPSADGEWTVRHTVAAWPAVCSGLRSEARNGRMLPYVIELDGQFCGQLTIGNVT
HGALRSAWIGYWVPSAATGGGVATGALALGLDHCFGPVMLHRVEATVRPENAASRAV
LAKVGFREEGLLRRYLEVDRAWRDHLLMAITVEEVYGSVASTLVRAGHASWP**
pVariable: nt sequence (IgE leader sequence is underlined)
(SEQ ID NO: 62)
ATGGACTGGACTTGGATTCTGTTTCTGGTCGCCGCCGCTACTAGGGTGCATTCAACT
CCTGTCAGGCCCCCACATACTCCCGATCCCCTGAATCTGAGAGGCCCACTGGACGGC
CCCCGGTGGAGGAGAGCAGAGCCTGCACAGTCCCGGCGCCCAGGCAGATCTCGGCC
TGGCGGCGCCCCACTGCGCTACCACAGGACAGGAGTGGGAATGAGCAGGACCGGAC
ACGGCTCCAGGCCTGTGCCCCCTGCCACCACAGTGGGCCTGGCCCTGCTGGCAGCAG
CCATCACCCTGTGGCTGGGCCTGGTGGCACAGTTCGGACAGATGATCACCGGCGGCT
CCGCCGACGGCTCCGCCGATTCTACAGGAAGGGTGCCAGACAGGCTGGCAGTGGTG
CGGGTCGAGACAGGAGAGTCTCTGTATGATGTGGCCGTGCGCGTGGCACCAAACGC
ACCAACAAGGCAGGTGGCAGACCGCATCAGGGAGCTGAATGGCCTGCAGACCCCTG
CCCTGGCAGTGGGACAGACACTGATCGCACCAGTGGGAAGAGGAAGGAAGAGGAG
ATCTCAGCAGACAGCCTGGGCCCCACGGACCAGCGGAATCGCAGGATGCGGAGCCG
GCGGCGTGGTCATGGCCATCGCCAGCGTGACCCTGGTGACCGATACACCAGGACGC
GTGCTGACAGGAGTGGCCGCCCTGGGCCTGATCCTGTTTGCATCCGCCACCTGGAGG
GCCAGGCCCAGACTGGCAATCACACCTGACGGCCTGGCAATCAGGGGATGGTTCAG
GACCCAGCTGCTGAGACACTCCAATATCAAGATCATCCGGATCGATGAGTTTCGGCG
CTACGGCAGACTGGTGCGGCTGCTGGAGATCGAGACAGTGTCTGGCGGCCTGCTGA
TCCTGAGCAGATGGGACCTGGGAACCGATCCCGTGGAGGTGCTGGACGCACTGACA
GCAGCAGGATATGCAGGAAGAGGACAGAGGAGAGGAAGGAAGCGGCGCTCTGCCG
TGAACAGGAGAGTGCCCCGGGTGCGCGACCTGGCCCCTCTGCTGCAGTTCAATAGG
CCACAGTTTGATACCAGCAAGCGGCGCCTGGGAGCCGCCCTGACAATCCAGGACCT
GAGGAGAATCGCAAAGAGGAGGACCCCTAGAGCCGCCTTCGACTACGCAGATGGCG
GAGCCGAGGATGAGCTGTCCATCGCCAGGGCCAGACAGGGCTTCAGAGACATCGAG
TTTCACCCTACAATCCTGCGGGATGTGACCACAGTGTGCGCAGGATGGAACGTGCTG
GGACAGCCAACCGTGCTGCCTTTCGGCATCGCACCAACAGGCTTTACCAGACTGATG
CACACAGAGGGCGAGATCGCAGGAGCCAGGGCCGCAGCAGCAGCAGGCATCCCCTT
TTCTCTGAGCACACTGGCCACCTGTGCCATCGAGGACCTGGTCATCGCCGTGCCTCA
GGGCAGGAAGTGGTTCCAGCTGTACATGTGGCGGGACCGCGATAGGAGCATGGCAC
TGGTGAGGAGAGTGGCAGCAGCAGGCTTTGACACAATGCTGGTGACCGTGGATGTG
CCAGTGGCAGGAGCAAGACTGAGGGATGTGCGGAACGGCATGTCCATCCCACCCGC
CCTGACACTGAGGACCGTGCTGGACGCAATGGGACACCCAAGGTGGTGGTTCGATC
TGCTGACCACAGAGCCCCTGGCCTTTGCCTCTCTGGACAGGTGGCCTGGAACAGTGG
GAGAGTATCTGAATACCGTGTTCGATCCCAGCCTGACATTTGACGATCTGGCCTGGA
TCAAGTCCCAGTGGCCTGGCAAGCTGGTGGTGAAGGGCATCCAGACCCTGGACGAT
GCCAGAGCCGTGGTGGACCGGGGCGTGGATGGAATCGTGCTGTCTAACCACGGCGG
CAGACAGCTGGACAGGGCACCAGTGCCTTTCCACCTGCTGCCACACGTGGCCCGGG
AGCTGGGCAAGCACACAGAGATCCTGGTGGACACCGGCATCATGAGCGGCGCCGAT
ATCGTGGCAGCAATCGCCCTGGGAGCAAGGTGCACCCTGATCGGCAGGGCCTACCT
GTATGGCCTGATGGCCGGCGGCGAGGCAGGAGTGAACAGGGCCATCGAGATCCTGC
AGACAGGCGTGATCCGCACCATGAGGCTGCTGGGCGTGACCTGTCTGGAGGAGCTG
TCTCCCCGCCACGTGACACAGCTGCGGCGCCTGGGACCAATCGGAGCACCCACCCG
CGGCAGGAAGAGGAGAAGCAGGCTGACACACCCAACCCCCTGCCCTGAGAATGGC
GAGACAATGATCGACCGGCGCAGGTCCGCCTGGAGATTCTCTGTGCCCCTGGTGTGC
CTGCTGGCAGGCCTGCTGCTGGCAGCAACCCACGGCGTGAGCGGCGGCACAGAGAT
CAGACGGTCCGACGCCCCTAGACTGGTGGATCTGGTGCGCAGGGCACAGGCAAGCG
TGAACAGGCTGGCAACCGAGAGGGAGGCCCTGACCACACGCATCGACAGCGTGCAC
GGCAGGTCCGTGGATACAGCCCTGGCAGCAATGCAGAGACGGTCCGCCAAGCTGGC
AGGAGTGGCAGCAATGAACCCTGTGCACGGACCAGGCCTGGTGGTGACCCTGCAGG
ACGCACAGAGAGATGCCAATGGCAGATTTCCCCGGGACGCATCCCCTGACGATCTG
GTGGTGCACCAGCAGGATATCGAGGCCGTGCTGAACGCACTGTGGAATGCAGGAGC
AGAGGCAATCCAGATGCAGGACCAGCGCATCATCGCCATGTCCATCGCCAGATGCG
TGGGCAACACCCTGCTGCTGAATGGCCGGACATACTCTCCTCCATATACCATCGCCG
CCATCGGCGATGCAGCAGCAATGCAGGCCGCCCTGGCAGCAGCCCCTCTGGTGACC
CTGTACAAGCAGTATGTGGTGCGGTTCGGCCTGGGCTACTGTGAGGAGGTGCACCCA
GACCTGCAGATCGTGGGCTATGCCGATCCCGTGCGCATGCACTTTGCACAGCCTGCA
GGACCACTGGACTACAGAGGAAGGAAGCGCAGGAGCGTGGCAGAGAGCTCCCTGA
ACCCAAGCCTGGTGTCCAGAATCTCTGCCTTCCTGCGGCCTGATTGGACCCGCACAG
TGAGGGCAAGACGGTTTGCAGCAGCAGGCCTGGTCATGCTGGCAGGAGTGGCCGCC
CTGAGATCCAATCCAGAGGACGATCGGTCTGAGGTGGTGGTGGCAGCACACGACCT
GAGGCCAGGAACAGCCCTGACCCCAGGCGATGTGCGCCTGGAGAAGAGGTCTGCCA
CCACACTGCCTGACGGCAGCCAGGCAGACCTGGATGCAGTGGTGGGCAGCACACTG
GCATCCCCAACCAGGAGGGGCGAGGTGCTGACCGACGTGAGACTGCTGGGCTCTCG
GCTGGCAGAGAGCACAGCAGGACCTGATGCAAGAATCGTGCCACTGCACCTGGCAG
ACAGCGCCCTGGTGGATCTGGTGCGGGTGGGCGACGTGGTGGATGTGCTGGCAGCA
CCAGTGACCGACTCCCCAGCCGCCCTGAGACTGCTGGCCACAGATGCCATCGTGGTG
CTGGTGTCCGCCCAGCAGAAGGCACAGGCAGCCGACTCTGATAGAGTGGTGCTGGT
GGCCCTGCCCGCCCGGCTGGCCAATACCGTGGCAGGAGCCGCCCTGGGACAGACCG
TGACACTGACCCTGCACAGGGGAAGGAAGAGACGGTCCGCCGTGGGACCACTGAGG
GTGTCTGCCGGCGTGATCAGACTGCGGCCCGTGCGCATGAGGGACGGAGTGCACTG
GTCTAGAATCCGGCTGGCAGATAGAGCACACCTGGAGCCATGGGAGCCTAGCGCCG
ACGGAGAGTGGACAGTGCGCCACACCGTGGCAGCATGGCCAGCCGTGTGCTCTGGC
CTGAGGAGCGAGGCAAGAAACGGAAGGATGCTGCCCTACGTGATCGAGCTGGATGG
CCAGTTCTGTGGCCAGCTGACAATCGGCAATGTGACCCACGGCGCCCTGAGGAGCG
CCTGGATCGGCTATTGGGTGCCTTCCGCCGCAACCGGCGGCGGCGTGGCAACCGGC
GCCCTGGCCCTGGGCCTGGACCACTGTTTCGGACCTGTGATGCTGCACAGGGTGGAG
GCAACCGTGAGGCCAGAGAACGCAGCCAGCCGCGCCGTGCTGGCCAAAGTGGGCTT
TAGGGAGGAGGGCCTGCTGCGCAGGTATCTGGAGGTGGACCGCGCCTGGAGGGATC
ACCTGCTGATGGCCATCACCGTGGAGGAGGTCTACGGGAGCGTCGCAAGCACACTG
GTCAGAGCAGGACACGCAAGCTGGCCTTGATAA
p5Ag: AA sequence
(SEQ ID NO: 63)
MDWTWILFLVAAATRVHSTDVSRKIRAWGRRLMIGTAAAVVLPGLVGLAGGAATAG
AFSRPGLPVEYLQVPSPSMGRDIKVQFQSGGNNSPAVYLLDGLRAQDDYNGWDINTPAF
EWYYQSGLSIVMPVGGQSSFYSDWYSPACGKAGCQTYKWETFLTSELPQWLSANRAV
KPTGSAAIGLSMAGSSAMILAAYHPQQFIYAGSLSALLDPSQGMGPSLIGLAMGDAGGY
KAADMWGPSSDPAWERNDPTQQIPKLVANNTRLWVYCGNGTPNELGGANIPAEFLENF
VRSSNLKFQDAYNAAGGHNAVFNFPPNGTHSWEYWGAQLNAMKGDLQSSLGAGRGR
KRRSTEQQWNFAGIEAAASAIQGNVTSIHSLLDEGKQSLTKLAAAWGGSGSEAYQGVQ
QKWDATATELNNALQNLARTISEAGQAMASTEGNVTGMFARGRKRRSIATTRDREGA
TMITFRLRLPCRTILRVFSRNPLVRGTDRLEAVVMLLAVTVSLLTIPFAAAAGTAVQDSR
SHVYAHQAQTRHPATATVIDHEGVIDSNTTATSAPPRTKITVPARWVVNGIERSGEVNA
KPGTKSGDRVGIWVDSAGQLVDEPAPPARAIADAALAALGLWLSVAAVAGALLALTRA
ILIRVRNASWQHDIDSLFCTQRRGRKRRSTTARDIMNAGVTCVGEHETLTAAAQYMRE
HDIGALPICGDDDRLHGMLTDRDIVIKGLAAGLDPNTATAGELARDSIYYVDANASIQE
MLNVMEEHQVRRVPVISEHRLVGIVTEADIARHLPEHAIVQFVKAICSPMALASRGRKR
RSLRLVVGALLLVLAFAGGYAVAACKTVTLTVDGTAMRVTTMKSRVIDIVEENGFSVD
DRDDLYPAAGVQVHDADTIVLRRSRPLQISLDGHDAKQVWTTASTVDEALAQLAMTDT
APAAASRASRVPLSGMALPVVSAKTVQLNDGGLVRTVHLPAPNVAGLLSAAGVPLLQS
DHVVPAATAPIVEGMQIQVTRNRIKKVTERLPLPPNARRVEDPEMNMSREVVEDPGVPG
TQDVTFAVAEVNGVETGRLPVANVVVTPAHEAVVRVGTKPGTEVPPVIDGSIWDAIAG
CEAGGNWAINTGNGYYGGVQFDQGTWEANGGLRYAPRADLATREEQIAVAEVTRLRQ
GWGAWPVCAARAGAR**
p5Ag: nt sequence (IgE leader is underlined)
(SEQ ID NO: 64)
ATGGACTGGACTTGGATTCTGTTTCTGGTCGCCGCCGCTACACGGGTGCACTCAACC
GATGTCAGCAGGAAGATTAGGGCTTGGGGCAGGAGACTGATGATCGGCACAGCCGC
CGCCGTGGTGCTGCCAGGCCTGGTGGGCCTGGCCGGCGGAGCCGCAACCGCAGGCG
CCTTCAGCAGGCCAGGCCTGCCAGTGGAGTACCTGCAGGTGCCTTCCCCATCTATGG
GCAGAGATATCAAGGTGCAGTTTCAGTCTGGCGGCAACAATAGCCCTGCCGTGTACC
TGCTGGATGGCCTGAGGGCCCAGGACGATTATAATGGCTGGGACATCAACACCCCA
GCCTTCGAGTGGTACTATCAGAGCGGCCTGTCCATCGTGATGCCAGTGGGCGGCCAG
AGCTCCTTTTACAGCGACTGGTATTCCCCAGCATGCGGCAAGGCAGGATGTCAGACA
TATAAGTGGGAGACATTCCTGACCTCTGAGCTGCCACAGTGGCTGAGCGCCAATAG
AGCCGTGAAGCCAACCGGCTCCGCCGCAATCGGCCTGTCTATGGCAGGCTCTAGCG
CCATGATCCTGGCAGCCTACCACCCTCAGCAGTTTATCTATGCAGGCAGCCTGTCCG
CCCTGCTGGACCCCAGCCAGGGAATGGGACCTTCCCTGATCGGCCTGGCAATGGGC
GACGCCGGCGGATACAAGGCAGCAGATATGTGGGGACCCTCCTCTGACCCTGCATG
GGAGCGGAACGATCCAACCCAGCAGATCCCCAAGCTGGTGGCCAACAATACACGCC
TGTGGGTGTATTGCGGAAACGGAACCCCAAATGAGCTGGGCGGCGCCAATATCCCT
GCCGAGTTCCTGGAGAATTTTGTGCGGAGCTCCAACCTGAAGTTCCAGGATGCATAC
AACGCAGCCGGCGGACACAACGCCGTGTTCAATTTTCCCCCTAACGGCACACACAG
CTGGGAGTATTGGGGCGCCCAGCTGAACGCAATGAAGGGCGACCTGCAGTCTAGCC
TGGGAGCAGGAAGGGGAAGGAAGCGGCGCAGCACCGAGCAGCAGTGGAATTTTGC
AGGAATCGAGGCAGCAGCATCCGCCATCCAGGGCAACGTGACATCTATCCACAGCC
TGCTGGATGAGGGCAAGCAGTCCCTGACCAAGCTGGCAGCAGCATGGGGCGGCTCT
GGCAGCGAGGCATACCAGGGAGTGCAGCAGAAGTGGGACGCAACCGCCACAGAGC
TGAACAATGCCCTGCAGAATCTGGCAAGGACCATCAGCGAGGCAGGACAGGCCATG
GCCTCCACAGAGGGCAACGTGACCGGCATGTTCGCCAGGGGCAGAAAGAGGAGAA
GCATCGCAACCACACGGGACCGCGAGGGAGCAACAATGATCACCTTCAGGCTGAGA
CTGCCCTGTCGGACAATCCTGCGCGTGTTTTCTAGGAATCCTCTGGTGAGAGGCACC
GATAGGCTGGAGGCAGTGGTCATGCTGCTGGCAGTGACAGTGAGCCTGCTGACCAT
CCCCTTTGCCGCAGCAGCAGGAACAGCAGTGCAGGACAGCAGGAGCCACGTGTACG
CACACCAGGCACAGACCAGGCACCCTGCAACCGCAACAGTGATCGATCACGAGGGC
GTGATCGACTCTAACACCACAGCCACAAGCGCCCCACCCCGCACAAAGATCACCGT
GCCCGCCAGATGGGTGGTGAATGGCATCGAGAGATCCGGCGAGGTGAACGCCAAGC
CTGGAACCAAGTCTGGCGACCGGGTGGGAATCTGGGTGGATAGCGCCGGACAGCTG
GTGGACGAGCCCGCCCCTCCAGCCAGGGCCATCGCAGACGCCGCCCTGGCCGCCCT
GGGCCTGTGGCTGTCCGTGGCCGCCGTGGCCGGCGCCCTGCTGGCCCTGACCCGGGC
CATCCTGATCCGGGTGCGCAATGCCTCCTGGCAGCACGATATCGACTCTCTGTTCTG
CACACAGAGGAGGGGAAGGAAGAGGAGATCTACCACAGCCCGCGATATCATGAAC
GCAGGAGTGACCTGCGTGGGAGAGCACGAGACACTGACAGCAGCAGCACAGTACA
TGAGGGAGCACGACATCGGCGCCCTGCCAATCTGCGGCGACGATGACAGGCTGCAC
GGCATGCTGACAGATAGAGACATCGTGATCAAGGGCCTGGCAGCAGGCCTGGACCC
CAATACCGCAACAGCAGGAGAGCTGGCCCGCGATTCCATCTACTATGTGGACGCCA
ATGCCTCTATCCAGGAGATGCTGAACGTGATGGAGGAGCACCAGGTGCGGAGGGTG
CCCGTGATCAGCGAGCACAGGCTGGTGGGCATCGTGACCGAGGCCGACATCGCCAG
ACACCTGCCTGAGCACGCCATCGTGCAGTTCGTGAAGGCCATCTGCTCCCCAATGGC
CCTGGCCTCTAGGGGCAGGAAAAGGAGATCCCTGAGGCTGGTGGTGGGCGCCCTGC
TGCTGGTGCTGGCCTTTGCCGGCGGCTACGCAGTGGCAGCCTGTAAGACCGTGACAC
TGACCGTGGATGGCACCGCCATGAGAGTGACCACAATGAAGAGCAGAGTGATCGAC
ATCGTGGAGGAGAACGGCTTCTCTGTGGATGACAGGGATGACCTGTATCCAGCAGC
AGGAGTGCAGGTGCACGATGCAGACACAATCGTGCTGCGGCGCAGCAGACCCCTGC
AGATCTCCCTGGATGGCCACGACGCCAAGCAAGTGTGGACCACAGCAAGCACCGTG
GATGAGGCCCTGGCACAGCTGGCAATGACAGACACCGCACCTGCAGCAGCCTCCAG
GGCCTCTAGAGTGCCACTGTCTGGAATGGCACTGCCAGTGGTGAGCGCCAAGACAG
TGCAGCTGAATGATGGCGGCCTGGTGAGAACCGTGCACCTGCCAGCCCCTAACGTG
GCAGGCCTGCTGAGCGCCGCAGGAGTGCCACTGCTGCAGTCCGACCACGTGGTGCC
TGCAGCAACCGCACCAATCGTGGAGGGAATGCAGATCCAGGTGACACGGAACCGCA
TCAAGAAGGTGACCGAGAGGCTGCCTCTGCCCCCTAATGCCAGGAGAGTGGAGGAC
CCAGAGATGAACATGTCTAGAGAGGTGGTGGAGGACCCCGGAGTGCCAGGAACCCA
GGACGTGACCTTCGCCGTGGCCGAAGTGAATGGAGTGGAGACAGGCCGGCTGCCTG
TGGCAAACGTGGTGGTGACCCCAGCACACGAGGCAGTGGTGCGCGTGGGAACAAAG
CCAGGAACCGAGGTGCCACCCGTGATCGATGGCTCCATCTGGGACGCAATCGCAGG
ATGTGAGGCCGGCGGAAATTGGGCCATCAACACAGGCAATGGCTACTATGGCGGCG
TGCAGTTTGATCAGGGAACCTGGGAGGCAAACGGCGGCCTGCGCTACGCCCCTAGG
GCCGACCTGGCAACCAGGGAGGAGCAGATCGCAGTGGCAGAGGTGACAAGGCTGA
GACAGGGGTGGGGAGCATGGCCCGTGTGCGCTGCAAGAGCTGGAGCAAGATGATAA
pEsx_R/S Null: AA Sequence
(SEQ ID NO: 65)
MDWTWILFLVAAATRVHSVSQSMYSYPAMTANVGDMAGYTGTTQSLGADIASERTA
PSRACQGDLGMSHQDWQAQWNQAMEALARAYRRCRRALRQIGVLERPVGDSSDCGTI
RVGSFRGRWLDPRHAGPATAADAGDRGRKRRSVADTIQVTPQMLRSTANDIQANMEQ
AMGIAKGYLANQENVMNPATWSGTGVVASHMTATEITNELNKVLTGGTRLAEGLVQA
AALMEGHEADSQTAFQALFGASHGSRGRKRRSTINYQFGDVDAHGAMIRAQAGLLEA
EHQAIVRDVLAAGDFWGGAGSVACQEFITQLGRNFQVIYEQANAHGQKVQAAGNNMA
QTDSAVGSSWARGRKRRSVSTPNTLNADFDLMRSVAGITDARNEEIRAMLQAFIGRMS
GVPPSVWGGLAAARFQDVVDRWNAESTRLYHVLHAIADTIRHNEAALREAGQIHARHI
AAAGGDLRGRKRRSTINYQFGDVDAHGAMIRAQAGSLEAEHQAIISDVLTASDFWGGA
GSAACQGFITQLGRNFQVIYEQANAHGQKVQAAGNNMAQTDSAVGSSWARGRKRRS
TEQQWNFAGIEAAASAIQGNVTSIHSLLDEGKQSLTKLAAAWGGSGSEAYQGVQQKWD
ATATELNNALQNLARTISEAGQAMASTEGNVTGMFARGRKRRSAEMKTDAATLAQEA
GNFERISGDLKTQIDQVESTAGSLQGQWRGAAGTAAQAAVVRFQEAANKQKQELDEIS
TNIRQAGVQYSRADEEQQQALSSQMGF*
pEsx_R/S Null: nt Sequence (IgE leader sequence is underlined)
(SEQ ID NO: 66)
ATGGACTGGACCTGGATTCTGTTCCTGGTGGCCGCCGCCACAAGAGTGCACTCTGTG
AGCCAGTCCATGTACAGCTATCCTGCCATGACCGCCAATGTGGGCGACATGGCCGG
CTACACAGGCACCACACAGTCCCTGGGAGCAGATATCGCATCTGAGAGGACCGCAC
CAAGCCGCGCCTGCCAGGGCGACCTGGGAATGTCCCACCAGGATTGGCAGGCCCAG
TGGAACCAGGCAATGGAGGCCCTGGCCAGGGCCTATAGGAGATGCAGGCGCGCCCT
GAGGCAGATCGGCGTGCTGGAGCGCCCAGTGGGCGACAGCTCCGATTGTGGCACCA
TCCGCGTGGGCAGCTTTAGGGGCAGATGGCTGGACCCAAGGCACGCAGGACCAGCA
ACAGCAGCAGACGCAGGCGATAGGGGAAGGAAGAGGAGAAGCGTGGCAGACACCA
TCCAGGTGACACCCCAGATGCTGCGCTCCACCGCCAATGATATCCAGGCCAACATG
GAGCAGGCCATGGGCATCGCCAAGGGCTACCTGGCCAACCAGGAGAATGTGATGAA
CCCTGCAACCTGGAGCGGAACAGGAGTGGTGGCATCCCACATGACCGCCACAGAGA
TCACAAATGAGCTGAACAAGGTGCTGACCGGCGGCACAAGGCTGGCAGAGGGACTG
GTGCAGGCAGCCGCCCTGATGGAGGGCCACGAGGCCGATTCTCAGACCGCATTCCA
GGCCCTGTTTGGAGCAAGCCACGGCTCCAGGGGCAGAAAGCGGCGCAGCACAATCA
ACTACCAGTTCGGCGACGTGGATGCACACGGAGCCATGATCAGGGCACAGGCAGGA
CTGCTGGAGGCAGAGCACCAGGCAATCGTGAGGGACGTGCTGGCAGCAGGCGATTT
TTGGGGAGGAGCAGGCTCCGTGGCATGCCAGGAGTTCATCACCCAGCTGGGCCGCA
ATTTTCAGGTCATCTACGAGCAGGCCAACGCACACGGACAGAAGGTGCAGGCAGCA
GGCAACAATATGGCCCAGACAGACTCTGCCGTGGGCTCTAGCTGGGCCAGGGGCAG
GAAGAGGAGATCTGTGAGCACCCCCAATACACTGAACGCCGACTTCGATCTGATGA
GAAGCGTGGCCGGCATCACCGATGCCAGGAATGAGGAGATCAGAGCCATGCTGCAG
GCCTTCATCGGAAGGATGAGCGGAGTGCCACCTTCCGTGTGGGGAGGACTGGCCGC
CGCCAGGTTTCAGGACGTGGTGGATAGATGGAATGCCGAGTCCACCCGGCTGTACC
ACGTGCTGCACGCCATCGCCGACACAATCCGCCACAACGAGGCCGCCCTGAGGGAG
GCCGGCCAGATCCACGCAAGGCACATCGCAGCAGCAGGAGGCGATCTGAGAGGAA
GAAAGCGGCGCAGCACCATCAACTATCAGTTTGGCGACGTGGACGCACACGGAGCA
ATGATTAGAGCACAGGCAGGCTCCCTGGAGGCCGAACACCAGGCCATCATCTCCGA
CGTGCTGACCGCCTCTGACTTCTGGGGAGGAGCAGGCAGCGCCGCATGTCAGGGCT
TCATCACACAGCTGGGCAGGAACTTCCAGGTCATCTACGAACAGGCTAATGCCCAT
GGCCAGAAAGTCCAGGCCGCAGGCAACAATATGGCACAGACCGACTCTGCCGTGGG
CTCCTCTTGGGCCAGGGGCAGAAAAAGGAGAAGCACAGAGCAGCAGTGGAATTTCG
CAGGAATCGAGGCAGCAGCATCCGCCATCCAGGGCAACGTGACCTCTATCCACAGC
CTGCTGGACGAGGGCAAGCAGTCTCTGACAAAGCTGGCAGCAGCATGGGGAGGCTC
CGGCTCTGAGGCATATCAGGGCGTGCAGCAGAAGTGGGATGCCACCGCCACAGAGC
TGAACAATGCCCTGCAGAATCTGGCCAGAACCATCTCTGAGGCAGGACAGGCAATG
GCAAGCACCGAGGGCAACGTGACAGGCATGTTCGCCAGGGGCAGGAAACGGCGCT
CCGCCGAGATGAAGACCGACGCAGCCACACTGGCACAGGAGGCAGGCAACTTTGAG
CGGATCTCTGGCGACCTGAAGACCCAGATCGATCAGGTGGAGTCCACAGCCGGCTC
TCTGCAGGGCCAGTGGAGAGGAGCAGCAGGAACCGCAGCACAGGCAGCAGTGGTG
CGGTTCCAGGAGGCCGCCAATAAGCAGAAGCAGGAGCTGGACGAGATCTCCACAAA
CATCAGACAGGCCGGCGTGCAGTATTCTCGGGCCGATGAGGAGCAGCAGCAGGCCC
TGAGCTCCCAGATGGGCTTTTGATGA
pEsxR: AA Sequence
(SEQ ID NO: 67)
MDWTWILFLVAAATRVHSSQIMYNYPAMMAHAGDMAGYAGTLQSLGADIASEQAV
LSSAWQGDTGITYQGWQTQWNQALEDLVRAYQSMSGTHESNTMAMLARDGAEAAK
WGG**
pEsxR: nt Sequence (IgE leader sequence is underlined)
(SEQ ID NO: 68)
ATGGACTGGACCTGGATTCTGTTCCTGGTGGCAGCAGCAACAAGGGTGCACAGCTCC
CAGATCATGTACAACTATCCCGCCATGATGGCACACGCAGGCGACATGGCAGGATA
CGCAGGCACCCTGCAGTCCCTGGGAGCAGATATCGCCTCTGAGCAGGCCGTGCTGTC
TAGCGCCTGGCAGGGCGACACCGGCATCACATACCAGGGCTGGCAGACACAGTGGA
ACCAGGCCCTGGAGGATCTGGTGAGGGCCTATCAGTCTATGAGCGGCACCCACGAG
TCTAATACAATGGCCATGCTGGCCAGAGACGGAGCAGAGGCAGCAAAGTGGGGAG
GATGATGA
-IgE leader sequence
SEQ ID NO: 69
MDWTWILFLVAAATRVHS

1. A nucleic acid molecule comprising a nucleic acid sequence encoding one or more tuberculosis (TB) antigen of: Rv3017c (EsxQ), Rv3020c (EsxS), Rv3019c (EsxR), Rv3891c (EsxD), Rv2346c (EsxO), Rv3445c (EsxU), Rv3619c (EsxV), Rv3875 (EsxA), Rv3874 (EsxB), Rv3136c (PPE51), Rv3615c (EspC), Rv1009c (Rpf B), Rv2034c, Rv2628c, Rv2719c, Rv0010c, Rv1872c, Rv0012, Rv0090c, Rv0095, Rv1886c (Ag85B), Rv1733c, and Rv2626c.

2. The nucleic acid molecule of claim 1, wherein the TB antigen of:
a. Rv3017c (EsxQ) is selected from the group consisting of: the amino acid sequence of SEQ ID NO:1 and a fragment of an amino acid having at least 60% identity over the entire length of the amino acid sequence of SEQ ID NO:1;
b. Rv3020c (EsxS) is selected from the group consisting of: the amino acid sequence of SEQ ID NO:3 and a fragment of an amino acid having at least 60% identity over the entire length of the amino acid sequence of SEQ ID NO:3;
c. Rv3019c (EsxR) is selected from the group consisting of: the amino acid sequence of SEQ ID NO:5 and a fragment of an amino acid having at least 60% identity over the entire length of the amino acid sequence of SEQ ID NO:5;
d. Rv3891c (EsxD) is selected from the group consisting of: the amino acid sequence of SEQ ID NO:7 and a fragment of an amino acid having at least 60% identity over the entire length of the amino acid sequence of SEQ ID NO:7;
e. Rv2346c (EsxO) is selected from the group consisting of: the amino acid sequence of SEQ ID NO:9 and a fragment of an amino acid having at least 60% identity over the entire length of the amino acid sequence of SEQ ID NO:9;
f. Rv3445c (EsxU) is selected from the group consisting of: the amino acid sequence of SEQ ID NO:11 and a fragment of an amino acid having at least 60% identity over the entire length of the amino acid sequence of SEQ ID NO:11;
g. Rv3619c (EsxV) is selected from the group consisting of: the amino acid sequence of SEQ ID NO:13 and a fragment of an amino acid having at least 60% identity over the entire length of the amino acid sequence of SEQ ID NO:13;
h. Rv3875 (EsxA) is selected from the group consisting of: the amino acid sequence of SEQ ID NO:15 and a fragment of an amino acid having at least 60% identity over the entire length of the amino acid sequence of SEQ ID NO:15;
i. Rv3874 (EsxB) is selected from the group consisting of: the amino acid sequence of SEQ ID NO:17 and a fragment of an amino acid having at least 60% identity over the entire length of the amino acid sequence of SEQ ID NO:17;
j. Rv3136c (PPE51) is selected from the group consisting of: the amino acid sequence of SEQ ID NO:19 and a fragment of an amino acid having at least 60% identity over the entire length of the amino acid sequence of SEQ ID NO:19;
k. Rv3615c (EspC) is selected from the group consisting of: the amino acid sequence of SEQ ID NO:21 and a fragment of an amino acid having at least 60% identity over the entire length of the amino acid sequence of SEQ ID NO:21;
l. Rv1009c (Rpf B) is selected from the group consisting of: the amino acid sequence of SEQ ID NO:23 and a fragment of an amino acid having at least 60% identity over the entire length of the amino acid sequence of SEQ ID NO:23;
m. Rv2034c is selected from the group consisting of: the amino acid sequence of SEQ ID NO:25 and a fragment of an amino acid having at least 60% identity over the entire length of the amino acid sequence of SEQ ID NO:25;
n. Rv2628c is selected from the group consisting of: the amino acid sequence of SEQ ID NO:27 and a fragment of an amino acid having at least 60% identity over the entire length of the amino acid sequence of SEQ ID NO:27;
o. Rv2719c is selected from the group consisting of: the amino acid sequence of SEQ ID NO:29 and a fragment of an amino acid having at least 60% identity over the entire length of the amino acid sequence of SEQ ID NO:29;
p. Rv0010c is selected from the group consisting of: the amino acid sequence of SEQ ID NO:31 and a fragment of an amino acid having at least 60% identity over the entire length of the amino acid sequence of SEQ ID NO:31;
q. Rv1872c is selected from the group consisting of: the amino acid sequence of SEQ ID NO:33 and a fragment of an amino acid having at least 60% identity over the entire length of the amino acid sequence of SEQ ID NO:33;
r. Rv0012 is selected from the group consisting of: the amino acid sequence of SEQ ID NO:35 and a fragment of an amino acid having at least 60% identity over the entire length of the amino acid sequence of SEQ ID NO:35;
s. Rv0090c is selected from the group consisting of: the amino acid sequence of SEQ ID NO:37 and a fragment of an amino acid having at least 60% identity over the entire length of the amino acid sequence of SEQ ID NO:37;
t. Rv0095 is selected from the group consisting of: the amino acid sequence of SEQ ID NO:39 and a fragment of an amino acid having at least 60% identity over the entire length of the amino acid sequence of SEQ ID NO:39;
u. Rv1886c (Ag85B) is selected from the group consisting of: the amino acid sequence of SEQ ID NO:41 and a fragment of an amino acid having at least 60% identity over the entire length of the amino acid sequence of SEQ ID NO:41;
v. Rv1733c is selected from the group consisting of: the amino acid sequence of SEQ ID NO:44 and a fragment of an amino acid having at least 60% identity over the entire length of the amino acid sequence of SEQ ID NO:44;
w. Rv2626c is selected from the group consisting of: the amino acid sequence of SEQ ID NO:46 and a fragment of an amino acid having at least 60% identity over the entire length of the amino acid sequence of SEQ ID NO:46;

3. The nucleic acid molecule of claim 1, wherein the nucleic acid sequence encoding the TB antigen of:
a. Rv3017c (EsxQ) is selected from the group consisting of: a nucleic acid sequence encoding the amino acid sequence of SEQ ID NO:1, a nucleic acid sequence encoding a fragment of an amino acid having at least 60% identity over the entire length of the amino acid sequence of SEQ ID NO: 1, the nucleic acid sequence of SEQ ID NO:2, a fragment of a nucleic acid sequence having at least 60% identity over the entire length of the nucleic acid sequence of SEQ ID NO:2, the nucleic acid sequence of SEQ ID NO:49, and a fragment of a nucleic acid sequence having at least 60% identity over the entire length of the nucleic acid sequence of SEQ ID NO:49;
b. Rv3020c (EsxS) is selected from the group consisting of: a nucleic acid sequence encoding the amino acid sequence of SEQ ID NO:3, a nucleic acid sequence encoding a fragment of an amino acid having at least 60% identity over the entire length of the amino acid sequence of SEQ ID NO:3, the nucleic acid sequence of SEQ ID NO:4, and a fragment of a nucleic acid sequence having at least 60% identity over the entire length of the nucleic acid sequence of SEQ ID NO:4;
c. Rv3019c (EsxR) is selected from the group consisting of: a nucleic acid sequence encoding the amino acid sequence of SEQ ID NO:5, a nucleic acid sequence encoding a fragment of an amino acid having at least 60% identity over the entire length of the amino acid sequence of SEQ ID NO:5, the nucleic acid sequence of SEQ ID NO:6, a fragment of a nucleic acid sequence having at least 60% identity over the entire length of the nucleic acid sequence of SEQ ID NO:6, the nucleic acid sequence of SEQ ID NO:56, a fragment of a nucleic acid sequence having at least 60% identity over the entire length of the nucleic acid sequence of SEQ ID NO:56;
d. Rv3891c (EsxD) is selected from the group consisting of: a nucleic acid sequence encoding the amino acid sequence of SEQ ID NO:7, a nucleic acid sequence encoding a fragment of an amino acid having at least 60% identity over the entire length of the amino acid sequence of SEQ ID NO:7, the nucleic acid sequence of SEQ ID NO:8, a fragment of a nucleic acid sequence having at least 60% identity over the entire length of the nucleic acid sequence of SEQ ID NO:8, the nucleic acid sequence of SEQ ID NO:50, and a fragment of a nucleic acid sequence having at least 60% identity over the entire length of the nucleic acid sequence of SEQ ID NO:50;
e. Rv2346c (EsxO) is selected from the group consisting of: a nucleic acid sequence encoding the amino acid sequence of SEQ ID NO:9, a nucleic acid sequence encoding a fragment of an amino acid having at least 60% identity over the entire length of the amino acid sequence of SEQ ID NO:9, the nucleic acid sequence of SEQ ID NO:10, a fragment of a nucleic acid sequence having at least 60% identity over the entire length of the nucleic acid sequence of SEQ ID NO:10, the nucleic acid sequence of SEQ ID NO:51, and a fragment of a nucleic acid sequence having at least 60% identity over the entire length of the nucleic acid sequence of SEQ ID NO:51;
f. Rv3445c (EsxU) is selected from the group consisting of: a nucleic acid sequence encoding the amino acid sequence of SEQ ID NO:11, a nucleic acid sequence encoding a fragment of an amino acid having at least 60% identity over the entire length of the amino acid sequence of SEQ ID NO:11, the nucleic acid sequence of SEQ ID NO:12, a fragment of a nucleic acid sequence having at least 60% identity over the entire length of the nucleic acid sequence of SEQ ID NO:12, the nucleic acid sequence of SEQ ID NO:52, and a fragment of a nucleic acid sequence having at least 60% identity over the entire length of the nucleic acid sequence of SEQ ID NO:52;
g. Rv3619c (EsxV) is selected from the group consisting of: a nucleic acid sequence encoding the amino acid sequence of SEQ ID NO:13, a nucleic acid sequence encoding a fragment of an amino acid having at least 60% identity over the entire length of the amino acid sequence of SEQ ID NO:13, the nucleic acid sequence of SEQ ID NO:14, a fragment of a nucleic acid sequence having at least 60% identity over the entire length of the nucleic acid sequence of SEQ ID NO:14, the nucleic acid sequence of SEQ ID NO:53, and a fragment of a nucleic acid sequence having at least 60% identity over the entire length of the nucleic acid sequence of SEQ ID NO:53;
h. Rv3875 (EsxA) is selected from the group consisting of: a nucleic acid sequence encoding the amino acid sequence of SEQ ID NO:15, a nucleic acid sequence encoding a fragment of an amino acid having at least 60% identity over the entire length of the amino acid sequence of SEQ ID NO: 15, the nucleic acid sequence of SEQ ID NO:16, a fragment of a nucleic acid sequence having at least 60% identity over the entire length of the nucleic acid sequence of SEQ ID NO:16, the nucleic acid sequence of SEQ ID NO:43, a fragment of a nucleic acid sequence having at least 60% identity over the entire length of the nucleic acid sequence of SEQ ID NO:43, the nucleic acid sequence of SEQ ID NO:54, and a fragment of a nucleic acid sequence having at least 60% identity over the entire length of the nucleic acid sequence of SEQ ID NO:54;
i. Rv3874 (EsxB) is selected from the group consisting of: a nucleic acid sequence encoding the amino acid sequence of SEQ ID NO:17, a nucleic acid sequence encoding a fragment of an amino acid having at least 60% identity over the entire length of the amino acid sequence of SEQ ID NO:17, the nucleic acid sequence of SEQ ID NO:18, a fragment of a nucleic acid sequence having at least 60% identity over the entire length of the nucleic acid sequence of SEQ ID NO:18, the nucleic acid sequence of SEQ ID NO:55, and a fragment of a nucleic acid sequence having at least 60% identity over the entire length of the nucleic acid sequence of SEQ ID NO:55;
j. Rv3136c (PPE51) is selected from the group consisting of: a nucleic acid sequence encoding the amino acid sequence of SEQ ID NO:19, a nucleic acid sequence encoding a fragment of an amino acid having at least 60% identity over the entire length of the amino acid sequence of SEQ ID NO:19, the nucleic acid sequence of SEQ ID NO:20, and a fragment of a nucleic acid sequence having at least 60% identity over the entire length of the nucleic acid sequence of SEQ ID NO:20;
k. Rv3615c (EspC) is selected from the group consisting of: a nucleic acid sequence encoding the amino acid sequence of SEQ ID NO:21, a nucleic acid sequence encoding a fragment of an amino acid having at least 60% identity over the entire length of the amino acid sequence of SEQ ID NO:21, the nucleic acid sequence of SEQ ID NO:22, and a fragment of a nucleic acid sequence having at least 60% identity over the entire length of the nucleic acid sequence of SEQ ID NO:22;
l. Rv1009c (Rpf B) is selected from the group consisting of: a nucleic acid sequence encoding the amino acid sequence of SEQ ID NO:23, a nucleic acid sequence encoding a fragment of an amino acid having at least 60% identity over the entire length of the amino acid sequence of SEQ ID NO:23, the nucleic acid sequence of SEQ ID NO:24, a fragment of a nucleic acid sequence having at least 60% identity over the entire length of the nucleic acid sequence of SEQ ID NO:24, the nucleic acid sequence of SEQ ID NO:48, and a fragment of a nucleic acid sequence having at least 60% identity over the entire length of the nucleic acid sequence of SEQ ID NO:48;
m. Rv2034c is selected from the group consisting of: a nucleic acid sequence encoding the amino acid sequence of SEQ ID NO:25, a nucleic acid sequence encoding a fragment of an amino acid having at least 60% identity over the entire length of the amino acid sequence of SEQ ID NO:25, the nucleic acid sequence of SEQ ID NO:26, and a fragment of a nucleic acid sequence having at least 60% identity over the entire length of the nucleic acid sequence of SEQ ID NO:26;
n. Rv2628c is selected from the group consisting of: a nucleic acid sequence encoding the amino acid sequence of SEQ ID NO:27, a nucleic acid sequence encoding a fragment of an amino acid having at least 60% identity over the entire length of the amino acid sequence of SEQ ID NO:27, the nucleic acid sequence of SEQ ID NO:28, and a fragment of a nucleic acid sequence having at least 60% identity over the entire length of the nucleic acid sequence of SEQ ID NO:28;
o. Rv2719c is selected from the group consisting of: a nucleic acid sequence encoding the amino acid sequence of SEQ ID NO:29, a nucleic acid sequence encoding a fragment of an amino acid having at least 60% identity over the entire length of the amino acid sequence of SEQ ID NO:29, the nucleic acid sequence of SEQ ID NO:30, and a fragment of a nucleic acid sequence having at least 60% identity over the entire length of the nucleic acid sequence of SEQ ID NO:30;
p. Rv0010c is selected from the group consisting of: a nucleic acid sequence encoding the amino acid sequence of SEQ ID NO:31, a nucleic acid sequence encoding a fragment of an amino acid having at least 60% identity over the entire length of the amino acid sequence of SEQ ID NO:31, the nucleic acid sequence of SEQ ID NO:32, and a fragment of a nucleic acid sequence having at least 60% identity over the entire length of the nucleic acid sequence of SEQ ID NO:32;
q. Rv1872c is selected from the group consisting of: a nucleic acid sequence encoding the amino acid sequence of SEQ ID NO:33, a nucleic acid sequence encoding a fragment of an amino acid having at least 60% identity over the entire length of the amino acid sequence of SEQ ID NO:33, the nucleic acid sequence of SEQ ID NO:34, and a fragment of a nucleic acid sequence having at least 60% identity over the entire length of the nucleic acid sequence of SEQ ID NO:34;
r. Rv0012 is selected from the group consisting of: a nucleic acid sequence encoding the amino acid sequence of SEQ ID NO:35, a nucleic acid sequence encoding a fragment of an amino acid having at least 60% identity over the entire length of the amino acid sequence of SEQ ID NO:35, the nucleic acid sequence of SEQ ID NO:36, and a fragment of a nucleic acid sequence having at least 60% identity over the entire length of the nucleic acid sequence of SEQ ID NO:36;
s. Rv0090c is selected from the group consisting of: a nucleic acid sequence encoding the amino acid sequence of SEQ ID NO:37, a nucleic acid sequence encoding a fragment of an amino acid having at least 60% identity over the entire length of the amino acid sequence of SEQ ID NO:37, the nucleic acid sequence of SEQ ID NO:38, and a fragment of a nucleic acid sequence having at least 60% identity over the entire length of the nucleic acid sequence of SEQ ID NO:38;
t. Rv0095 is selected from the group consisting of: a nucleic acid sequence encoding the amino acid sequence of SEQ ID NO:39, a nucleic acid sequence encoding a fragment of an amino acid having at least 60% identity over the entire length of the amino acid sequence of SEQ ID NO:39, the nucleic acid sequence of SEQ ID NO:40, and a fragment of a nucleic acid sequence having at least 60% identity over the entire length of the nucleic acid sequence of SEQ ID NO:40;
u. Rv1886c (Ag85B) is selected from the group consisting of: a nucleic acid sequence encoding the amino acid sequence of SEQ ID NO:41, a nucleic acid sequence encoding a fragment of an amino acid having at least 60% identity over the entire length of the amino acid sequence of SEQ ID NO:41, the nucleic acid sequence of SEQ ID NO:42, and a fragment of a nucleic acid sequence having at least 60% identity over the entire length of the nucleic acid sequence of SEQ ID NO:42;
v. Rv1733c is selected from the group consisting of: a nucleic acid sequence encoding the amino acid sequence of SEQ ID NO:44, a nucleic acid sequence encoding a fragment of an amino acid having at least 60% identity over the entire length of the amino acid sequence of SEQ ID NO:44, the nucleic acid sequence of SEQ ID NO:45, and a fragment of a nucleic acid sequence having at least 60% identity over the entire length of the nucleic acid sequence of SEQ ID NO:45; and
w. Rv2626c is selected from the group consisting of: a nucleic acid sequence encoding the amino acid sequence of SEQ ID NO:46, a nucleic acid sequence encoding a fragment of an amino acid having at least 60% identity over the entire length of the amino acid sequence of SEQ ID NO:46, the nucleic acid sequence of SEQ ID NO:47, and a fragment of a nucleic acid sequence having at least 60% identity over the entire length of the nucleic acid sequence of SEQ ID NO:47.

4. The nucleic acid molecule of claim 1 comprising one or more of:
a. a nucleic acid sequence encoding one or more TB antigen of: Rv3017c (EsxQ), Rv3020c (EsxS), Rv3019c (EsxR), Rv3891c (EsxD), Rv2346c (EsxO), Rv3445c (EsxU), Rv3619c (EsxV), Rv3875 (EsxA), and Rv3874 (EsxB);
b. a nucleic acid sequence encoding one or more TB antigen of: Rv3136c (PPE51), Rv3615c (EspC), Rv1009c (Rpf B), Rv2034c, and Rv2628c;
c. a nucleic acid sequence encoding one or more TB antigen of: Rv2719c, Rv0010c, Rv1872c, Rv0012, Rv0090c, and Rv0095;
d. a nucleic acid sequence encoding one or more TB antigen of: Rv1886c (Ag85B), Rv3875 (EsxA), Rv1733c, Rv2626c, and Rv1009c (Rpf B);
e. a nucleic acid sequence encoding one or more TB antigen of: Rv3017c (EsxQ), Rv3891c (EsxD), Rv2346c (EsxO), Rv3445c (EsxU), Rv3619c (EsxV), Rv3875 (EsxA), and Rv3874 (EsxB); and
f. a nucleic acid sequence encoding the TB antigen of Rv3019c (EsxR).

5. The nucleic acid molecule of claim 4, wherein the nucleic acid molecule comprises one or more of:
a. a nucleic acid sequence encoding the combination of: SEQ ID NO: 1, SEQ ID NO:3, SEQ ID NO:5, SEQ ID NO:7, SEQ ID NO:9, SEQ ID NO: 11, SEQ ID NO:13, SEQ ID NO:15, and SEQ ID NO:17;
b. a nucleic acid sequence encoding the combination of: SEQ ID NO: 19, SEQ ID NO:21, SEQ ID NO:23, SEQ ID NO:25, and SEQ ID NO:27;
c. a nucleic acid sequence encoding the combination of: SEQ ID NO:29, SEQ ID NO:31, SEQ ID NO:33, SEQ ID NO:35, SEQ ID NO:37, and SEQ ID NO:39;
d. a nucleic acid sequence encoding the combination of: SEQ ID NO:41, SEQ ID NO: 15, SEQ ID NO:44, SEQ ID NO:46, and SEQ ID NO:23;
e. a nucleic acid sequence encoding the combination of: SEQ ID NO: 1, SEQ ID NO:7, SEQ ID NO:9, SEQ ID NO: 11, SEQ ID NO: 13, SEQ ID NO: 15, and SEQ ID NO:17; and
f. a nucleic acid sequence encoding the TB antigen of SEQ ID NO:5.

6. The nucleic acid molecule of claim 4, wherein the nucleic acid molecule comprises one or more of:
a. a nucleic acid sequence comprising the combination of: SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18;
b. a nucleic acid sequence comprising the combination of: SEQ ID NO:20, SEQ ID NO:22, SEQ ID NO:24, SEQ ID NO:26, and SEQ ID NO:28;
c. a nucleic acid sequence comprising the combination of: SEQ ID NO:30, SEQ ID NO:32, SEQ ID NO:34, SEQ ID NO:36, SEQ ID NO:38, and SEQ ID NO:40;
d. a nucleic acid sequence comprising the combination of: SEQ ID NO:42, SEQ ID NO:43, SEQ ID NO:45, SEQ ID NO:47, and SEQ ID NO:48;
e. a nucleic acid sequence comprising the combination of: SEQ ID NO:49, SEQ ID NO:50, SEQ ID NO:51, SEQ ID NO:52, SEQ ID NO:53, SEQ ID NO:54, and SEQ ID NO:55; and
f. a nucleic acid sequence comprising SEQ ID NO:56.

7. The nucleic acid molecule of claim 1, wherein the nucleic acid molecule comprises an expression vector.

8. The nucleic acid molecule of claim 1, wherein the encoded TB antigen is operably linked to at least one of: a start codon, an IgE leader sequence, a furin cleavage site, and a stop codon.

9. An immunogenic composition comprising the nucleic acid molecule of claim 1.

10. The immunogenic composition of claim 9 further comprising a pharmaceutically acceptable excipient, an adjuvant, or a combination thereof.

11. The immunogenic composition of claim 9 further comprising nucleic acid sequences that encode one or more proteins selected from the group consisting of: IL-12, IL-15, and IL28.

12. A method of inducing an immune response against tuberculosis in a subject in need thereof, the method comprising administering the immunogenic composition of claim 9.

13. The method of claim 12, further comprising an electroporation step.

14. The method of claim 12, wherein the immune response is protective or therapeutic.


SEQUENCE LISTING (69 sequences)

SEQ ID NO: 1 - protein, 120 aa, synthetic construct
VSQSMYSYPAMTANVGDMAGYTGTTQSLGADIASERTAPSRACQGDLGMSHQDWQAQWNQAMEALARAYRRCRRALRQIGVLERPVGDSSDCGTIRVGSFRGRWLDPRHAGPATAADAGD

SEQ ID NO: 2 - DNA, 360 bp, synthetic construct
  source 1..360: mol_type = other DNA
gtctcacagagcatgtattcttaccccgcaatgaccgccaatgtgggcgacatggccggctacacaggcaccacacagtccctgggagcagatatcgcatccgagaggaccgcaccctctcgcgcctgccagggcgacctgggcatgtctcaccaggattggcaggcccagtggaaccaggccatggaggccctggccagagcctataggagatgcaggcgcgccctgaggcagatcggcgtgctggagcgccctgtgggcgacagctccgattgtggcaccatcagagtgggctctttcaggggcagatggctggacccacggcacgcaggaccagcaacagcagcagacgcaggcgat

SEQ ID NO: 3 - protein, 96 aa, synthetic construct
SLLDAHIPQLIASHTAFAAKAGLMRHTIGQAEQQAMSAQAFHQGESAAAFQGAHARFVAAAAKVNTLLDIAQANLGEAAGTYVAADAAAASSYTGF

SEQ ID NO: 4 - DNA, 288 bp, synthetic construct
  source 1..288: mol_type = other DNA
agcctgctggatgcccacatcccacagctgatcgcatcccacaccgccttcgccgcaaaggcaggcctgatgcgccacacaatcggacaggcagagcagcaggcaatgtctgcccaggcatttcaccagggagagagcgccgcagcattccagggagcacacgcaaggtttgtggcagcagccgccaaagtgaataccctgctggacatcgcacaggcaaacctgggagaggcagcaggcacctacgtggccgccgatgccgccgccgcctcctcttatacaggcttt

SEQ ID NO: 5 - protein, 95 aa, synthetic construct
SQIMYNYPAMMAHAGDMAGYAGTLQSLGADIASEQAVLSSAWQGDTGITYQGWQTQWNQALEDLVRAYQSMSGTHESNTMAMLARDGAEAAKWGG

SEQ ID NO: 6 - DNA, 285 bp, synthetic construct
  source 1..285: mol_type = other DNA
tcccagatcatgtacaactatcccgccatgatggcacacgcaggcgacatggcaggatacgcaggcaccctgcagtctctgggcgccgatatcgcaagcgagcaggccgtgctgtctagcgcctggcagggcgacaccggcatcacataccagggctggcagacacagtggaatcaggccctggaggatctggtgcgcgcctatcagtctatgagcggcacccacgagagcaacacaatggcaatgctggccagggacggagcagaggcagcaaagtggggcggc

SEQ ID NO: 7 - protein, 107 aa, synthetic construct
VADTIQVTPQMLRSTANDIQANMEQAMGIAKGYLANQENVMNPATWSGTGVVASHMTATEITNELNKVLTGGTRLAEGLVQAAALMEGHEADSQTAFQALFGASHGS

SEQ ID NO: 8 - DNA, 321 bp, synthetic construct
  source 1..321: mol_type = other DNA
gtggccgacaccatccaggtgacacctcagatgctgagatctaccgccaatgatatccaggccaacatggagcaggccatgggcatcgccaagggctacctggccaaccaggagaatgtgatgaacccagcaacctggtccggaacaggagtggtggcatctcacatgaccgccacagagatcacaaatgagctgaacaaggtgctgaccggcggcacaaggctggcagagggcctggtgcaggccgccgccctgatggagggacacgaggcagactcccagaccgcattccaggccctgtttggcgcctctcacggcagc

SEQ ID NO: 9 - protein, 93 aa, synthetic construct
TINYQFGDVDAHGAMIRAQAGLLEAEHQAIVRDVLAAGDFWGGAGSVACQEFITQLGRNFQVIYEQANAHGQKVQAAGNNMAQTDSAVGSSWA

SEQ ID NO: 10 - DNA, 279 bp, synthetic construct
  source 1..279: mol_type = other DNA
acaatcaactaccagttcggcgacgtggatgcacacggagcaatgatcagagcacaggcaggcctgctggaggcagagcaccaggcaatcgtgcgggacgtgctggcagcaggcgatttttggggcggcgccggctccgtggcatgccaggagttcatcacccagctgggccgcaattttcaggtcatctacgagcaggccaacgcacacggacagaaggtgcaggcagcaggcaacaatatggcacagacagacagcgccgtgggctcctcttgggcc

SEQ ID NO: 11 - protein, 105 aa, synthetic construct
VSTPNTLNADFDLMRSVAGITDARNEEIRAMLQAFIGRMSGVPPSVWGGLAAARFQDVVDRWNAESTRLYHVLHAIADTIRHNEAALREAGQIHARHIAAAGGDL

SEQ ID NO: 12 - DNA, 315 bp, synthetic construct
  source 1..315: mol_type = other DNA
gtgtctacccccaatacactgaacgccgacttcgatctgatgagatccgtggccggcatcaccgatgccaggaatgaggagatcagagccatgctgcaggccttcatcggaaggatgtctggagtgccccctagcgtgtggggcggcctggcagccgccaggtttcaggacgtggtggatagatggaatgccgagagcacccggctgtaccacgtgctgcacgccatcgccgacacaatcaggcacaacgaggccgccctgagggaggccggccagatccacgccagacacatcgcagcagccggcggcgatctg

SEQ ID NO: 13 - protein, 93 aa, synthetic construct
TINYQFGDVDAHGAMIRAQAGSLEAEHQAIISDVLTASDFWGGAGSAACQGFITQLGRNFQVIYEQANAHGQKVQAAGNNMAQTDSAVGSSWA

SEQ ID NO: 14 - DNA, 279 bp, synthetic construct
  source 1..279: mol_type = other DNA
accatcaactatcagtttggcgacgtggacgcccatggagcaatgatcagggcacaggcaggcagcctggaggccgaacaccaggccatcatcagcgacgtgctgaccgcctctgacttctggggcggcgccggctccgccgcatgtcagggcttcatcacacagctgggccggaacttccaggtcatctacgaacaggctaatgcccatggccagaaagtccaggccgcaggcaacaatatggcccagaccgacagcgccgtgggcagctcctgggcc

SEQ ID NO: 15 - protein, 94 aa, synthetic construct
TEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 16 - DNA, 282 bp, synthetic construct
  source 1..282: mol_type = other DNA
acagagcagcagtggaatttcgcaggaatcgaggcagcagcatctgccatccagggcaacgtgacctccatccactctctgctggacgagggcaagcagagcctgacaaagctggcagcagcatggggcggcagcggctccgaggcatatcagggagtgcagcagaagtgggatgccaccgccacagagctgaacaatgccctgcagaatctggcaaggaccatcagcgaggcaggacaggccatggcctccaccgagggcaacgtgacaggcatgttcgcc

SEQ ID NO: 17 - protein, 99 aa, synthetic construct
AEMKTDAATLAQEAGNFERISGDLKTQIDQVESTAGSLQGQWRGAAGTAAQAAVVRFQEAANKQKQELDEISTNIRQAGVQYSRADEEQQQALSSQMGF

SEQ ID NO: 18 - DNA, 297 bp, synthetic construct
  source 1..297: mol_type = other DNA
gccgagatgaagaccgacgcagccacactggcacaggaggcaggcaactttgagaggatctccggcgacctgaagacccagatcgatcaggtggagagcacagcaggctccctgcagggccagtggaggggcgccgcaggaaccgcagcacaggcagcagtggtgaggtttcaggaggcagccaataagcagaagcaggagctggatgagatcagcacaaacatcaggcaggccggcgtccagtattccagggcagatgaagagcagcagcaggcactgtcaagccagatgggattt

SEQ ID NO: 19 - protein, 379 aa, synthetic construct
DFALLPPEVNSARMYTGPGAGSLLAAAGGWDSLAAELATTAEAYGSVLSGLAALHWRGPAAESMAVTAAPYIGWLYTTAEKTQQTAIQARAAALAFEQAYAMTLPPPVVAANRIQLLALIATNFFGQNTAAIAATEAQYAEMWAQDAAAMYGYATASAAAALLTPFSPPRQTTNPAGLTAQAAAVSQATDPLSLLIETVTQALQALTIPSFIPEDFTFLDAIFAGYATVGVTQDVESFVAGTIGAESNLGLLNVGDENPAEVTPGDFGIGELVSATSPGGGVSASGAGGAASVGNTVLASVGRANSIGQLSVPPSWAAPSTRPVSALSPAGLTTLPGTDVAEHGMPGVPGVPVAAGRASGVLPRYGVRLTVMAHPPAAG

SEQ ID NO: 20 - DNA, 1137 bp, synthetic construct
  source 1..1137: mol_type = other DNA
gatttcgctctgctgcctcctgaagtcaactcagcaagaatgtacaccggacctggagcaggctctctgctggcagcagccggcggatgggacagcctggccgccgagctggccaccacagccgaggcctatggctccgtgctgtctggcctggccgccctgcactggaggggacctgcagccgagagcatggcagtgaccgcagcaccatacatcggatggctgtataccacagccgagaagacacagcagaccgcaatccaggccagagccgccgccctggccttcgagcaggcctacgccatgacactgccccctccagtggtggccgccaatagaatccagctgctggccctgatcgccaccaacttctttggccagaatacagcagcaatcgcagcaaccgaggcacagtatgcagagatgtgggcacaggacgcagcagcaatgtacggctatgccacagcatccgccgcagccgccctgctgacccccttttctccccctagacagaccacaaaccctgcaggcctgacagcacaggcagcagccgtgagccaggccaccgatccactgtccctgctgatcgagacagtgacacaggccctgcaggccctgacaatcccatccttcatccccgaggacttcacctttctggatgccatctttgcaggatacgcaacagtgggagtgacccaggacgtggagagcttcgtggcaggaaccatcggagcagagtccaacctgggcctgctgaatgtgggcgacgagaacccagccgaggtgacacccggcgattttggcatcggcgagctggtgagcgccacctcccccggcggcggcgtgagcgcctccggagccggcggagccgcaagcgtgggaaataccgtgctggcaagcgtgggcagagccaactccatcggccagctgtctgtgccacccagctgggcagcaccttccacacggccagtgtctgccctgagcccagcaggcctgaccacactgcctggaaccgacgtggcagagcacggcatgccaggagtgcctggagtgccagtggcagccggcagagccagcggcgtgctgccacgctacggcgtgaggctgaccgtgatggcacaccctccagcagcagga

SEQ ID NO: 21 - protein, 102 aa, synthetic construct
TENLTVQPERLGVLASHHDNAAVDASSGVEAAAGLGESVAITHGPYCSQFNDTLNVYLTAHNALGSSLHTAGVDLAKSLRIAAKIYSEADEAWRKAIDGLFT

SEQ ID NO: 22 - DNA, 306 bp, synthetic construct
  source 1..306: mol_type = other DNA
acagagaatctgaccgtgcagccagagcggctgggcgtgctggcaagccaccacgacaacgccgccgtggatgccagctccggagtggaggcagcagcaggcctgggagagtctgtggccatcacacacggcccatactgcagccagttcaacgacaccctgaacgtgtacctgaccgcccacaatgcactgggctctagcctgcacaccgcaggagtggatctggcaaagtccctgagaatcgccgccaagatctactctgaggcagacgaggcatggaggaaggcaatcgatggcctgttcacc

SEQ ID NO: 23 - protein, 361 aa, synthetic construct
LRLVVGALLLVLAFAGGYAVAACKTVTLTVDGTAMRVTTMKSRVIDIVEENGFSVDDRDDLYPAAGVQVHDADTIVLRRSRPLQISLDGHDAKQVWTTASTVDEALAQLAMTDTAPAAASRASRVPLSGMALPVVSAKTVQLNDGGLVRTVHLPAPNVAGLLSAAGVPLLQSDHVVPAATAPIVEGMQIQVTRNRIKKVTERLPLPPNARRVEDPEMNMSREVVEDPGVPGTQDVTFAVAEVNGVETGRLPVANVVVTPAHEAVVRVGTKPGTEVPPVIDGSIWDAIAGCEAGGNWAINTGNGYYGGVQFDQGTWEANGGLRYAPRADLATREEQIAVAEVTRLRQGWGAWPVCAARAGAR

SEQ ID NO: 24 - DNA, 1083 bp, synthetic construct
  source 1..1083: mol_type = other DNA
ctgaggctggtggtgggcgccctgctgctggtgctggcctttgccggcggctatgcagtggcagcctgtaagaccgtgacactgaccgtggacggaaccgcaatgagggtgaccacaatgaagagcagagtgatcgatatcgtggaggagaacggcttttccgtggacgatagggacgatctgtaccctgcagcaggagtgcaggtgcacgacgccgatacaatcgtgctgaggagatctaggccactgcagatcagcctggacggccacgatgccaagcaagtgtggaccacagcatccaccgtggacgaggccctggcacagctggcaatgacagataccgcacctgcagcagcctcccgggcctctcgcgtgccactgtctggaatggcactgccagtggtgagcgccaagacagtgcagctgaatgacggcggcctggtgaggaccgtgcacctgcctgcaccaaacgtggcaggcctgctgagcgccgcaggagtgccactgctgcagtccgatcacgtggtgcctgcagcaaccgcaccaatcgtggagggaatgcagatccaggtgacacggaatcgcatcaagaaggtgaccgagcgcctgccactgccccctaatgcaaggcgcgtggaggaccctgagatgaacatgtctagggaggtggtggaggaccccggagtgcctggcacacaggatgtgaccttcgccgtggccgaagtgaatggagtggagacaggccgcctgcctgtggcaaacgtggtggtgaccccagcacacgaggcagtggtgagggtgggaacaaagccaggaaccgaggtgccacccgtgatcgacggctccatctgggatgcaatcgcaggatgcgaggccggcggaaattgggccatcaacacaggcaatggctactatggcggcgtgcagtttgaccagggaacctgggaggcaaacggcggcctgagatacgcccctcgggccgatctggcaacacgcgaggagcagatcgcagtggcagaggtgaccaggctgagacagggatggggcgcctggcccgtgtgcgcagccagagccggcgccagg

SEQ ID NO: 25 - protein, 107 aa, synthetic construct
VSTYRSPDRAWQALADGTRRAIVERLAHGPLAVGELARDLPVSRPAVSQHLKVLKTARLVCDRPAGTRRVYQLDPTGLAALRTDLDRFWTRALTGYAQLIDSEGDDT

SEQ ID NO: 26 - DNA, 321 bp, synthetic construct
  source 1..321: mol_type = other DNA
gtgagcacatatcgcagccccgacagggcatggcaggccctggcagatggaaccaggagagccatcgtggagagactggcacacggacctctggcagtgggagagctggccagagatctgcccgtgtcccggcctgccgtgtctcagcacctgaaggtgctgaagacagcaaggctggtgtgcgacaggccagcaggaaccaggcgcgtgtaccagctggaccccacaggcctggccgccctgcgcaccgacctggatagattctggacacgggccctgaccggatatgcacagctgatcgactccgagggcgacgataca

SEQ ID NO: 27 - protein, 119 aa, synthetic construct
STQRPRHSGIRAVGPYAWAGRCGRIGRWGVHQEAMMNLAIWHPRKVQSATIYQVTDRSHDGRTARVPGDEITSTVSGWLSELGTQSPLADELARAVRIGDWPAAYAIGEHLSVEIAVAV

SEQ ID NO: 28 - DNA, 357 bp, synthetic construct
  source 1..357: mol_type = other DNA
tctacccagaggccaagacactctggcatcagggcagtgggaccatacgcatgggcaggcaggtgtggaaggatcggccggtggggcgtgcaccaggaggcaatgatgaacctggccatctggcaccctcgcaaggtgcagtccgccacaatctatcaggtgaccgacaggagccacgatggaaggacagccagggtgccaggcgacgagatcacaagcaccgtgtccggatggctgtccgagctgggaacccagtctcctctggcagatgagctggccagagccgtgcggatcggcgattggcccgccgcctacgctattggggaacacctgtccgtggagattgctgtcgcagtg

SEQ ID NO: 29 - protein, 164 aa, synthetic construct
TPVRPPHTPDPLNLRGPLDGPRWRRAEPAQSRRPGRSRPGGAPLRYHRTGVGMSRTGHGSRPVPPATTVGLALLAAAITLWLGLVAQFGQMITGGSADGSADSTGRVPDRLAVVRVETGESLYDVAVRVAPNAPTRQVADRIRELNGLQTPALAVGQTLIAPVG

SEQ ID NO: 30 - DNA, 492 bp, synthetic construct
  source 1..492: mol_type = other DNA
actcctgtcaggcccccacatactcccgatcccctgaatctgagaggcccactggacggcccccggtggaggagagcagagcctgcacagtcccggcgcccaggcagatctcggcctggcggcgccccactgcgctaccacaggacaggagtgggaatgagcaggaccggacacggctccaggcctgtgccccctgccaccacagtgggcctggccctgctggcagcagccatcaccctgtggctgggcctggtggcacagttcggacagatgatcaccggcggctccgccgacggctccgccgattctacaggaagggtgccagacaggctggcagtggtgcgggtcgagacaggagagtctctgtatgatgtggccgtgcgcgtggcaccaaacgcaccaacaaggcaggtggcagaccgcatcagggagctgaatggcctgcagacccctgccctggcagtgggacagacactgatcgcaccagtggga

SEQ ID NO: 31 - protein, 140 aa, synthetic construct
QQTAWAPRTSGIAGCGAGGVVMAIASVTLVTDTPGRVLTGVAALGLILFASATWRARPRLAITPDGLAIRGWFRTQLLRHSNIKIIRIDEFRRYGRLVRLLEIETVSGGLLILSRWDLGTDPVEVLDALTAAGYAGRGQR

SEQ ID NO: 32 - DNA, 420 bp, synthetic construct
  source 1..420: mol_type = other DNA
cagcagacagcctgggccccacggaccagcggaatcgcaggatgcggagccggcggcgtggtcatggccatcgccagcgtgaccctggtgaccgatacaccaggacgcgtgctgacaggagtggccgccctgggcctgatcctgtttgcatccgccacctggagggccaggcccagactggcaatcacacctgacggcctggcaatcaggggatggttcaggacccagctgctgagacactccaatatcaagatcatccggatcgatgagtttcggcgctacggcagactggtgcggctgctggagatcgagacagtgtctggcggcctgctgatcctgagcagatgggacctgggaaccgatcccgtggaggtgctggacgcactgacagcagcaggatatgcaggaagaggacagagg

SEQ ID NO: 33 - protein, 413 aa, synthetic construct
AVNRRVPRVRDLAPLLQFNRPQFDTSKRRLGAALTIQDLRRIAKRRTPRAAFDYADGGAEDELSIARARQGFRDIEFHPTILRDVTTVCAGWNVLGQPTVLPFGIAPTGFTRLMHTEGEIAGARAAAAAGIPFSLSTLATCAIEDLVIAVPQGRKWFQLYMWRDRDRSMALVRRVAAAGFDTMLVTVDVPVAGARLRDVRNGMSIPPALTLRTVLDAMGHPRWWFDLLTTEPLAFASLDRWPGTVGEYLNTVFDPSLTFDDLAWIKSQWPGKLVVKGIQTLDDARAVVDRGVDGIVLSNHGGRQLDRAPVPFHLLPHVARELGKHTEILVDTGIMSGADIVAAIALGARCTLIGRAYLYGLMAGGEAGVNRAIEILQTGVIRTMRLLGVTCLEELSPRHVTQLRRLGPIGAPT

SEQ ID NO: 34 - DNA, 1239 bp, synthetic construct
  source 1..1239: mol_type = other DNA
gccgtgaacaggagagtgccccgggtgcgcgacctggcccctctgctgcagttcaataggccacagtttgataccagcaagcggcgcctgggagccgccctgacaatccaggacctgaggagaatcgcaaagaggaggacccctagagccgccttcgactacgcagatggcggagccgaggatgagctgtccatcgccagggccagacagggcttcagagacatcgagtttcaccctacaatcctgcgggatgtgaccacagtgtgcgcaggatggaacgtgctgggacagccaaccgtgctgcctttcggcatcgcaccaacaggctttaccagactgatgcacacagagggcgagatcgcaggagccagggccgcagcagcagcaggcatccccttttctctgagcacactggccacctgtgccatcgaggacctggtcatcgccgtgcctcagggcaggaagtggttccagctgtacatgtggcgggaccgcgataggagcatggcactggtgaggagagtggcagcagcaggctttgacacaatgctggtgaccgtggatgtgccagtggcaggagcaagactgagggatgtgcggaacggcatgtccatcccacccgccctgacactgaggaccgtgctggacgcaatgggacacccaaggtggtggttcgatctgctgaccacagagcccctggcctttgcctctctggacaggtggcctggaacagtgggagagtatctgaataccgtgttcgatcccagcctgacatttgacgatctggcctggatcaagtcccagtggcctggcaagctggtggtgaagggcatccagaccctggacgatgccagagccgtggtggaccggggcgtggatggaatcgtgctgtctaaccacggcggcagacagctggacagggcaccagtgcctttccacctgctgccacacgtggcccgggagctgggcaagcacacagagatcctggtggacaccggcatcatgagcggcgccgatatcgtggcagcaatcgccctgggagcaaggtgcaccctgatcggcagggcctacctgtatggcctgatggccggcggcgaggcaggagtgaacagggccatcgagatcctgcagacaggcgtgatccgcaccatgaggctgctgggcgtgacctgtctggaggagctgtctccccgccacgtgacacagctgcggcgcctgggaccaatcggagcacccacc

SEQ ID NO: 35 - protein, 261 aa, synthetic construct
RLTHPTPCPENGETMIDRRRSAWRFSVPLVCLLAGLLLAATHGVSGGTEIRRSDAPRLVDLVRRAQASVNRLATEREALTTRIDSVHGRSVDTALAAMQRRSAKLAGVAAMNPVHGPGLVVTLQDAQRDANGRFPRDASPDDLVVHQQDIEAVLNALWNAGAEAIQMQDQRIIAMSIARCVGNTLLLNGRTYSPPYTIAAIGDAAAMQAALAAAPLVTLYKQYVVRFGLGYCEEVHPDLQIVGYADPVRMHFAQPAGPLDY

SEQ ID NO: 36 - DNA, 783 bp, synthetic construct
  source 1..783: mol_type = other DNA
aggctgacacacccaaccccctgccctgagaatggcgagacaatgatcgaccggcgcaggtccgcctggagattctctgtgcccctggtgtgcctgctggcaggcctgctgctggcagcaacccacggcgtgagcggcggcacagagatcagacggtccgacgcccctagactggtggatctggtgcgcagggcacaggcaagcgtgaacaggctggcaaccgagagggaggccctgaccacacgcatcgacagcgtgcacggcaggtccgtggatacagccctggcagcaatgcagagacggtccgccaagctggcaggagtggcagcaatgaaccctgtgcacggaccaggcctggtggtgaccctgcaggacgcacagagagatgccaatggcagatttccccgggacgcatcccctgacgatctggtggtgcaccagcaggatatcgaggccgtgctgaacgcactgtggaatgcaggagcagaggcaatccagatgcaggaccagcgcatcatcgccatgtccatcgccagatgcgtgggcaacaccctgctgctgaatggccggacatactctcctccatataccatcgccgccatcggcgatgcagcagcaatgcaggccgccctggcagcagcccctctggtgaccctgtacaagcagtatgtggtgcggttcggcctgggctactgtgaggaggtgcacccagacctgcagatcgtgggctatgccgatcccgtgcgcatgcactttgcacagcctgcaggaccactggactac

SEQ ID NO: 37 - protein, 218 aa, synthetic construct
VAESSLNPSLVSRISAFLRPDWTRTVRARRFAAAGLVMLAGVAALRSNPEDDRSEVVVAAHDLRPGTALTPGDVRLEKRSATTLPDGSQADLDAVVGSTLASPTRRGEVLTDVRLLGSRLAESTAGPDARIVPLHLADSALVDLVRVGDVVDVLAAPVTDSPAALRLLATDAIVVLVSAQQKAQAADSDRVVLVALPARLANTVAGAALGQTVTLTLH

SEQ ID NO: 38 - DNA, 654 bp, synthetic construct
  source 1..654: mol_type = other DNA
gtggcagagagctccctgaacccaagcctggtgtccagaatctctgccttcctgcggcctgattggacccgcacagtgagggcaagacggtttgcagcagcaggcctggtcatgctggcaggagtggccgccctgagatccaatccagaggacgatcggtctgaggtggtggtggcagcacacgacctgaggccaggaacagccctgaccccaggcgatgtgcgcctggagaagaggtctgccaccacactgcctgacggcagccaggcagacctggatgcagtggtgggcagcacactggcatccccaaccaggaggggcgaggtgctgaccgacgtgagactgctgggctctcggctggcagagagcacagcaggacctgatgcaagaatcgtgccactgcacctggcagacagcgccctggtggatctggtgcgggtgggcgacgtggtggatgtgctggcagcaccagtgaccgactccccagccgccctgagactgctggccacagatgccatcgtggtgctggtgtccgcccagcagaaggcacaggcagccgactctgatagagtggtgctggtggccctgcccgcccggctggccaataccgtggcaggagccgccctgggacagaccgtgacactgaccctgcac

SEQ ID NO: 39 - protein, 202 aa, synthetic construct
AVGPLRVSAGVIRLRPVRMRDGVHWSRIRLADRAHLEPWEPSADGEWTVRHTVAAWPAVCSGLRSEARNGRMLPYVIELDGQFCGQLTIGNVTHGALRSAWIGYWVPSAATGGGVATGALALGLDHCFGPVMLHRVEATVRPENAASRAVLAKVGFREEGLLRRYLEVDRAWRDHLLMAITVEEVYGSVASTLVRAGHASWP

SEQ ID NO: 40 - DNA, 606 bp, synthetic construct
  source 1..606: mol_type = other DNA
gccgtgggaccactgagggtgtctgccggcgtgatcagactgcggcccgtgcgcatgagggacggagtgcactggtctagaatccggctggcagatagagcacacctggagccatgggagcctagcgccgacggagagtggacagtgcgccacaccgtggcagcatggccagccgtgtgctctggcctgaggagcgaggcaagaaacggaaggatgctgccctacgtgatcgagctggatggccagttctgtggccagctgacaatcggcaatgtgacccacggcgccctgaggagcgcctggatcggctattgggtgccttccgccgcaaccggcggcggcgtggcaaccggcgccctggccctgggcctggaccactgtttcggacctgtgatgctgcacagggtggaggcaaccgtgaggccagagaacgcagccagccgcgccgtgctggccaaagtgggctttagggaggagggcctgctgcgcaggtatctggaggtggaccgcgcctggagggatcacctgctgatggccatcaccgtggaggaggtctacgggagcgtcgcaagcacactggtcagagcaggacacgcaagctggcct

SEQ ID NO: 41 - protein, 324 aa, synthetic construct
TDVSRKIRAWGRRLMIGTAAAVVLPGLVGLAGGAATAGAFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 42 - DNA, 972 bp, synthetic construct
  source 1..972: mol_type = other DNA
accgatgtcagcaggaagattagggcttggggcaggagactgatgatcggcacagccgccgccgtggtgctgccaggcctggtgggcctggccggcggagccgcaaccgcaggcgccttcagcaggccaggcctgccagtggagtacctgcaggtgccttccccatctatgggcagagatatcaaggtgcagtttcagtctggcggcaacaatagccctgccgtgtacctgctggatggcctgagggcccaggacgattataatggctgggacatcaacaccccagccttcgagtggtactatcagagcggcctgtccatcgtgatgccagtgggcggccagagctccttttacagcgactggtattccccagcatgcggcaaggcaggatgtcagacatataagtgggagacattcctgacctctgagctgccacagtggctgagcgccaatagagccgtgaagccaaccggctccgccgcaatcggcctgtctatggcaggctctagcgccatgatcctggcagcctaccaccctcagcagtttatctatgcaggcagcctgtccgccctgctggaccccagccagggaatgggaccttccctgatcggcctggcaatgggcgacgccggcggatacaaggcagcagatatgtggggaccctcctctgaccctgcatgggagcggaacgatccaacccagcagatccccaagctggtggccaacaatacacgcctgtgggtgtattgcggaaacggaaccccaaatgagctgggcggcgccaatatccctgccgagttcctggagaattttgtgcggagctccaacctgaagttccaggatgcatacaacgcagccggcggacacaacgccgtgttcaattttccccctaacggcacacacagctgggagtattggggcgcccagctgaacgcaatgaagggcgacctgcagtctagcctgggagcagga

SEQ ID NO: 43 - DNA, 282 bp, synthetic construct
  source 1..282: mol_type = other DNA
accgagcagcagtggaattttgcaggaatcgaggcagcagcatccgccatccagggcaacgtgacatctatccacagcctgctggatgagggcaagcagtccctgaccaagctggcagcagcatggggcggctctggcagcgaggcataccagggagtgcagcagaagtgggacgcaaccgccacagagctgaacaatgccctgcagaatctggcaaggaccatcagcgaggcaggacaggccatggcctccacagagggcaacgtgaccggcatgttcgcc

SEQ ID NO: 44 - protein, 209 aa, synthetic construct
IATTRDREGATMITFRLRLPCRTILRVFSRNPLVRGTDRLEAVVMLLAVTVSLLTIPFAAAAGTAVQDSRSHVYAHQAQTRHPATATVIDHEGVIDSNTTATSAPPRTKITVPARWVVNGIERSGEVNAKPGTKSGDRVGIWVDSAGQLVDEPAPPARAIADAALAALGLWLSVAAVAGALLALTRAILIRVRNASWQHDIDSLFCTQR

SEQ ID NO: 45 - DNA, 627 bp, synthetic construct
  source 1..627: mol_type = other DNA
atcgcaaccacacgggaccgcgagggagcaacaatgatcaccttcaggctgagactgccctgtcggacaatcctgcgcgtgttttctaggaatcctctggtgagaggcaccgataggctggaggcagtggtcatgctgctggcagtgacagtgagcctgctgaccatcccctttgccgcagcagcaggaacagcagtgcaggacagcaggagccacgtgtacgcacaccaggcacagaccaggcaccctgcaaccgcaacagtgatcgatcacgagggcgtgatcgactctaacaccacagccacaagcgccccaccccgcacaaagatcaccgtgcccgccagatgggtggtgaatggcatcgagagatccggcgaggtgaacgccaagcctggaaccaagtctggcgaccgggtgggaatctgggtggatagcgccggacagctggtggacgagcccgcccctccagccagggccatcgcagacgccgccctggccgccctgggcctgtggctgtccgtggccgccgtggccggcgccctgctggccctgacccgggccatcctgatccgggtgcgcaatgcctcctggcagcacgatatcgactctctgttctgcacacagagg

SEQ ID NO: 46 - protein, 142 aa, synthetic construct
TTARDIMNAGVTCVGEHETLTAAAQYMREHDIGALPICGDDDRLHGMLTDRDIVIKGLAAGLDPNTATAGELARDSIYYVDANASIQEMLNVMEEHQVRRVPVISEHRLVGIVTEADIARHLPEHAIVQFVKAICSPMALAS

SEQ ID NO: 47 - DNA, 426 bp, synthetic construct
  source 1..426: mol_type = other DNA
accacagcccgcgatatcatgaacgcaggagtgacctgcgtgggagagcacgagacactgacagcagcagcacagtacatgagggagcacgacatcggcgccctgccaatctgcggcgacgatgacaggctgcacggcatgctgacagatagagacatcgtgatcaagggcctggcagcaggcctggaccccaataccgcaacagcaggagagctggcccgcgattccatctactatgtggacgccaatgcctctatccaggagatgctgaacgtgatggaggagcaccaggtgcggagggtgcccgtgatcagcgagcacaggctggtgggcatcgtgaccgaggccgacatcgccagacacctgcctgagcacgccatcgtgcagttcgtgaaggccatctgctccccaatggccctggcctct

SEQ ID NO: 48 - DNA, 1083 bp, synthetic construct
  source 1..1083: mol_type = other DNA
ctgaggctggtggtgggcgccctgctgctggtgctggcctttgccggcggctacgcagtggcagcctgtaagaccgtgacactgaccgtggatggcaccgccatgagagtgaccacaatgaagagcagagtgatcgacatcgtggaggagaacggcttctctgtggatgacagggatgacctgtatccagcagcaggagtgcaggtgcacgatgcagacacaatcgtgctgcggcgcagcagacccctgcagatctccctggatggccacgacgccaagcaagtgtggaccacagcaagcaccgtggatgaggccctggcacagctggcaatgacagacaccgcacctgcagcagcctccagggcctctagagtgccactgtctggaatggcactgccagtggtgagcgccaagacagtgcagctgaatgatggcggcctggtgagaaccgtgcacctgccagcccctaacgtggcaggcctgctgagcgccgcaggagtgccactgctgcagtccgaccacgtggtgcctgcagcaaccgcaccaatcgtggagggaatgcagatccaggtgacacggaaccgcatcaagaaggtgaccgagaggctgcctctgccccctaatgccaggagagtggaggacccagagatgaacatgtctagagaggtggtggaggaccccggagtgccaggaacccaggacgtgaccttcgccgtggccgaagtgaatggagtggagacaggccggctgcctgtggcaaacgtggtggtgaccccagcacacgaggcagtggtgcgcgtgggaacaaagccaggaaccgaggtgccacccgtgatcgatggctccatctgggacgcaatcgcaggatgtgaggccggcggaaattgggccatcaacacaggcaatggctactatggcggcgtgcagtttgatcagggaacctgggaggcaaacggcggcctgcgctacgcccctagggccgacctggcaaccagggaggagcagatcgcagtggcagaggtgacaaggctgagacaggggtggggagcatggcccgtgtgcgctgcaagagctggagcaaga

SEQ ID NO: 49 - DNA, 360 bp, synthetic construct
  source 1..360: mol_type = other DNA
gtgagccagtccatgtacagctatcctgccatgaccgccaatgtgggcgacatggccggctacacaggcaccacacagtccctgggagcagatatcgcatctgagaggaccgcaccaagccgcgcctgccagggcgacctgggaatgtcccaccaggattggcaggcccagtggaaccaggcaatggaggccctggccagggcctataggagatgcaggcgcgccctgaggcagatcggcgtgctggagcgcccagtgggcgacagctccgattgtggcaccatccgcgtgggcagctttaggggcagatggctggacccaaggcacgcaggaccagcaacagcagcagacgcaggcgat

SEQ ID NO: 50 - DNA, 321 bp, synthetic construct
  source 1..321: mol_type = other DNA
gtggcagacaccatccaggtgacaccccagatgctgcgctccaccgccaatgatatccaggccaacatggagcaggccatgggcatcgccaagggctacctggccaaccaggagaatgtgatgaaccctgcaacctggagcggaacaggagtggtggcatcccacatgaccgccacagagatcacaaatgagctgaacaaggtgctgaccggcggcacaaggctggcagagggactggtgcaggcagccgccctgatggagggccacgaggccgattctcagaccgcattccaggccctgtttggagcaagccacggctcc

SEQ ID NO: 51 - DNA, 279 bp, synthetic construct
  source 1..279: mol_type = other DNA
acaatcaactaccagttcggcgacgtggatgcacacggagccatgatcagggcacaggcaggactgctggaggcagagcaccaggcaatcgtgagggacgtgctggcagcaggcgatttttggggaggagcaggctccgtggcatgccaggagttcatcacccagctgggccgcaattttcaggtcatctacgagcaggccaacgcacacggacagaaggtgcaggcagcaggcaacaatatggcccagacagactctgccgtgggctctagctgggcc

SEQ ID NO: 52 - DNA, 315 bp, synthetic construct
  source 1..315: mol_type = other DNA
gtgagcacccccaatacactgaacgccgacttcgatctgatgagaagcgtggccggcatcaccgatgccaggaatgaggagatcagagccatgctgcaggccttcatcggaaggatgagcggagtgccaccttccgtgtggggaggactggccgccgccaggtttcaggacgtggtggatagatggaatgccgagtccacccggctgtaccacgtgctgcacgccatcgccgacacaatccgccacaacgaggccgccctgagggaggccggccagatccacgcaaggcacatcgcagcagcaggaggcgatctg

SEQ ID NO: 53 - DNA, 279 bp, synthetic construct
  source 1..279: mol_type = other DNA
accatcaactatcagtttggcgacgtggacgcacacggagcaatgattagagcacaggcaggctccctggaggccgaacaccaggccatcatctccgacgtgctgaccgcctctgacttctggggaggagcaggcagcgccgcatgtcagggcttcatcacacagctgggcaggaacttccaggtcatctacgaacaggctaatgcccatggccagaaagtccaggccgcaggcaacaatatggcacagaccgactctgccgtgggctcctcttgggcc

SEQ ID NO: 54 - DNA, 282 bp, synthetic construct
  source 1..282: mol_type = other DNA
acagagcagcagtggaatttcgcaggaatcgaggcagcagcatccgccatccagggcaacgtgacctctatccacagcctgctggacgagggcaagcagtctctgacaaagctggcagcagcatggggaggctccggctctgaggcatatcagggcgtgcagcagaagtgggatgccaccgccacagagctgaacaatgccctgcagaatctggccagaaccatctctgaggcaggacaggcaatggcaagcaccgagggcaacgtgacaggcatgttcgcc

SEQ ID NO: 55 - DNA, 297 bp, synthetic construct
  source 1..297: mol_type = other DNA
gccgagatgaagaccgacgcagccacactggcacaggaggcaggcaactttgagcggatctctggcgacctgaagacccagatcgatcaggtggagtccacagccggctctctgcagggccagtggagaggagcagcaggaaccgcagcacaggcagcagtggtgcggttccaggaggccgccaataagcagaagcaggagctggacgagatctccacaaacatcagacaggccggcgtgcagtattctcgggccgatgaggagcagcagcaggccctgagctcccagatgggcttt

SEQ ID NO: 56 - DNA, 285 bp, synthetic construct
  source 1..285: mol_type = other DNA
tcccagatcatgtacaactatcccgccatgatggcacacgcaggcgacatggcaggatacgcaggcaccctgcagtccctgggagcagatatcgcctctgagcaggccgtgctgtctagcgcctggcagggcgacaccggcatcacataccagggctggcagacacagtggaaccaggccctggaggatctggtgagggcctatcagtctatgagcggcacccacgagtctaatacaatggccatgctggccagagacggagcagaggcagcaaagtggggagga

SEQ ID NO: 57 - protein, 976 aa, synthetic construct
MDWTWILFLVAAATRVHSVSQSMYSYPAMTANVGDMAGYTGTTQSLGADIASERTAPSRACQGDLGMSHQDWQAQWNQAMEALARAYRRCRRALRQIGVLERPVGDSSDCGTIRVGSFRGRWLDPRHAGPATAADAGDRGRKRRSSLLDAHIPQLIASHTAFAAKAGLMRHTIGQAEQQAMSAQAFHQGESAAAFQGAHARFVAAAAKVNTLLDIAQANLGEAAGTYVAADAAAASSYTGFRGRKRRSSQIMYNYPAMMAHAGDMAGYAGTLQSLGADIASEQAVLSSAWQGDTGITYQGWQTQWNQALEDLVRAYQSMSGTHESNTMAMLARDGAEAAKWGGRGRKRRSVADTIQVTPQMLRSTANDIQANMEQAMGIAKGYLANQENVMNPATWSGTGVVASHMTATEITNELNKVLTGGTRLAEGLVQAAALMEGHEADSQTAFQALFGASHGSRGRKRRSTINYQFGDVDAHGAMIRAQAGLLEAEHQAIVRDVLAAGDFWGGAGSVACQEFITQLGRNFQVIYEQANAHGQKVQAAGNNMAQTDSAVGSSWARGRKRRSVSTPNTLNADFDLMRSVAGITDARNEEIRAMLQAFIGRMSGVPPSVWGGLAAARFQDVVDRWNAESTRLYHVLHAIADTIRHNEAALREAGQIHARHIAAAGGDLRGRKRRSTINYQFGDVDAHGAMIRAQAGSLEAEHQAIISDVLTASDFWGGAGSAACQGFITQLGRNFQVIYEQANAHGQKVQAAGNNMAQTDSAVGSSWARGRKRRSTEQQWNFAGIEAAASAIQGNVTSIHSLLDEGKQSLTKLAAAWGGSGSEAYQGVQQKWDATATELNNALQNLARTISEAGQAMASTEGNVTGMFARGRKRRSAEMKTDAATLAQEAGNFERISGDLKTQIDQVESTAGSLQGQWRGAAGTAAQAAVVRFQEAANKQKQELDEISTNIRQAGVQYSRADEEQQQALSSQMGF

SEQ ID NO: 58 - DNA, 2934 bp, synthetic construct
  source 1..2934: mol_type = other DNA
atggactggacttggattctgttcctggtcgccgccgcaactagggtgcatagcgtctcacagagcatgtattcttaccccgcaatgaccgccaatgtgggcgacatggccggctacacaggcaccacacagtccctgggagcagatatcgcatccgagaggaccgcaccctctcgcgcctgccagggcgacctgggcatgtctcaccaggattggcaggcccagtggaaccaggccatggaggccctggccagagcctataggagatgcaggcgcgccctgaggcagatcggcgtgctggagcgccctgtgggcgacagctccgattgtggcaccatcagagtgggctctttcaggggcagatggctggacccacggcacgcaggaccagcaacagcagcagacgcaggcgatcggggaaggaagaggagatctagcctgctggatgcccacatcccacagctgatcgcatcccacaccgccttcgccgcaaaggcaggcctgatgcgccacacaatcggacaggcagagcagcaggcaatgtctgcccaggcatttcaccagggagagagcgccgcagcattccagggagcacacgcaaggtttgtggcagcagccgccaaagtgaataccctgctggacatcgcacaggcaaacctgggagaggcagcaggcacctacgtggccgccgatgccgccgccgcctcctcttatacaggctttaggggcagaaagcggcgcagctcccagatcatgtacaactatcccgccatgatggcacacgcaggcgacatggcaggatacgcaggcaccctgcagtctctgggcgccgatatcgcaagcgagcaggccgtgctgtctagcgcctggcagggcgacaccggcatcacataccagggctggcagacacagtggaatcaggccctggaggatctggtgcgcgcctatcagtctatgagcggcacccacgagagcaacacaatggcaatgctggccagggacggagcagaggcagcaaagtggggcggccggggccgcaagaggagatccgtggccgacaccatccaggtgacacctcagatgctgagatctaccgccaatgatatccaggccaacatggagcaggccatgggcatcgccaagggctacctggccaaccaggagaatgtgatgaacccagcaacctggtccggaacaggagtggtggcatctcacatgaccgccacagagatcacaaatgagctgaacaaggtgctgaccggcggcacaaggctggcagagggcctggtgcaggccgccgccctgatggagggacacgaggcagactcccagaccgcattccaggccctgtttggcgcctctcacggcagcaggggcaggaaacggcgctctacaatcaactaccagttcggcgacgtggatgcacacggagcaatgatcagagcacaggcaggcctgctggaggcagagcaccaggcaatcgtgcgggacgtgctggcagcaggcgatttttggggcggcgccggctccgtggcatgccaggagttcatcacccagctgggccgcaattttcaggtcatctacgagcaggccaacgcacacggacagaaggtgcaggcagcaggcaacaatatggcacagacagacagcgccgtgggctcctcttgggccaggggcaggaagaggagatccgtgtctacccccaatacactgaacgccgacttcgatctgatgagatccgtggccggcatcaccgatgccaggaatgaggagatcagagccatgctgcaggccttcatcggaaggatgtctggagtgccccctagcgtgtggggcggcctggcagccgccaggtttcaggacgtggtggatagatggaatgccgagagcacccggctgtaccacgtgctgcacgccatcgccgacacaatcaggcacaacgaggccgccctgagggaggccggccagatccacgccagacacatcgcagcagccggcggcgatctgaggggaagaaagcggcgctctaccatcaactatcagtttggcgacgtggacgcccatggagcaatgatcagggcacaggcaggcagcctggaggccgaacaccaggccatcatcagcgacgtgctgaccgcctctgacttctggggcggcgccggctccgccgcatgtcagggcttcatcacacagctgggccggaacttccaggtcatctacgaacaggctaatgcccatggccagaaagtccaggccgcaggcaacaatatggcccagaccgacagcgccgtgggcagctcctgggccaggggcagaaaaaggagatccacagagcagcagtggaatttcgcaggaatcgaggcagcagcatctgccatccagggcaacgtgacctccatccactctctgctggacgagggcaagcagagcctgacaaagctggcagcagcatggggcggcagcggctccgaggcatatcagggagtgcagcagaagtgggatgccaccgccacagagctgaacaatgccctgcagaatctggcaaggaccatcagcgaggcaggacaggccatggcctccaccgagggcaacgtgacaggcatgttcgccaggggcaggaagcggcgcagcgccgagatgaagaccgacgcagccacactggcacaggaggcaggcaactttgagaggatctccggcgacctgaagacccagatcgatcaggtggagagcacagcaggctccctgcagggccagtggaggggcgccgcaggaaccgcagcacaggcagcagtggtgaggtttcaggaggcagccaataagcagaagcaggagctggatgagatcagcacaaacatcaggcaggccggcgtccagtattccagggcagatgaagagcagcagcaggcactgtcaagccagatgggattttgataa

SEQ ID NO: 59 - protein, 1114 aa, synthetic construct
MDWTWILFLVAAATRVHSDFALLPPEVNSARMYTGPGAGSLLAAAGGWDSLAAELATTAEAYGSVLSGLAALHWRGPAAESMAVTAAPYIGWLYTTAEKTQQTAIQARAAALAFEQAYAMTLPPPVVAANRIQLLALIATNFFGQNTAAIAATEAQYAEMWAQDAAAMYGYATASAAAALLTPFSPPRQTTNPAGLTAQAAAVSQATDPLSLLIETVTQALQALTIPSFIPEDFTFLDAIFAGYATVGVTQDVESFVAGTIGAESNLGLLNVGDENPAEVTPGDFGIGELVSATSPGGGVSASGAGGAASVGNTVLASVGRANSIGQLSVPPSWAAPSTRPVSALSPAGLTTLPGTDVAEHGMPGVPGVPVAAGRASGVLPRYGVRLTVMAHPPAAGRGRKRRSTENLTVQPERLGVLASHHDNAAVDASSGVEAAAGLGESVAITHGPYCSQFNDTLNVYLTAHNALGSSLHTAGVDLAKSLRIAAKIYSEADEAWRKAIDGLFTRGRKRRSLRLVVGALLLVLAFAGGYAVAACKTVTLTVDGTAMRVTTMKSRVIDIVEENGFSVDDRDDLYPAAGVQVHDADTIVLRRSRPLQISLDGHDAKQVWTTASTVDEALAQLAMTDTAPAAASRASRVPLSGMALPVVSAKTVQLNDGGLVRTVHLPAPNVAGLLSAAGVPLLQSDHVVPAATAPIVEGMQIQVTRNRIKKVTERLPLPPNARRVEDPEMNMSREVVEDPGVPGTQDVTFAVAEVNGVETGRLPVANVVVTPAHEAVVRVGTKPGTEVPPVIDGSIWDAIAGCEAGGNWAINTGNGYYGGVQFDQGTWEANGGLRYAPRADLATREEQIAVAEVTRLRQGWGAWPVCAARAGARRGRKRRSVSTYRSPDRAWQALADGTRRAIVERLAHGPLAVGELARDLPVSRPAVSQHLKVLKTARLVCDRPAGTRRVYQLDPTGLAALRTDLDRFWTRALTGYAQLIDSEGDDTRGRKRRSSTQRPRHSGIRAVGPYAWAGRCGRIGRWGVHQEAMMNLAIWHPRKVQSATIYQVTDRSHDGRTARVPGDEITSTVSGWLSELGTQSPLADELARAVRIGDWPAAYAIGEHLSVEIAVAV

SEQ ID NO: 60 - DNA, 3348 bp, synthetic construct
  source 1..3348: mol_type = other DNA
atggattggacatggattctgttcctggtcgccgccgccacacgggtgcattccgatttcgctctgctgcctcctgaagtcaactcagcaagaatgtacaccggacctggagcaggctctctgctggcagcagccggcggatgggacagcctggccgccgagctggccaccacagccgaggcctatggctccgtgctgtctggcctggccgccctgcactggaggggacctgcagccgagagcatggcagtgaccgcagcaccatacatcggatggctgtataccacagccgagaagacacagcagaccgcaatccaggccagagccgccgccctggccttcgagcaggcctacgccatgacactgccccctccagtggtggccgccaatagaatccagctgctggccctgatcgccaccaacttctttggccagaatacagcagcaatcgcagcaaccgaggcacagtatgcagagatgtgggcacaggacgcagcagcaatgtacggctatgccacagcatccgccgcagccgccctgctgacccccttttctccccctagacagaccacaaaccctgcaggcctgacagcacaggcagcagccgtgagccaggccaccgatccactgtccctgctgatcgagacagtgacacaggccctgcaggccctgacaatcccatccttcatccccgaggacttcacctttctggatgccatctttgcaggatacgcaacagtgggagtgacccaggacgtggagagcttcgtggcaggaaccatcggagcagagtccaacctgggcctgctgaatgtgggcgacgagaacccagccgaggtgacacccggcgattttggcatcggcgagctggtgagcgccacctcccccggcggcggcgtgagcgcctccggagccggcggagccgcaagcgtgggaaataccgtgctggcaagcgtgggcagagccaactccatcggccagctgtctgtgccacccagctgggcagcaccttccacacggccagtgtctgccctgagcccagcaggcctgaccacactgcctggaaccgacgtggcagagcacggcatgccaggagtgcctggagtgccagtggcagccggcagagccagcggcgtgctgccacgctacggcgtgaggctgaccgtgatggcacaccctccagcagcaggaaggggacgcaagaggagatctacagagaatctgaccgtgcagccagagcggctgggcgtgctggcaagccaccacgacaacgccgccgtggatgccagctccggagtggaggcagcagcaggcctgggagagtctgtggccatcacacacggcccatactgcagccagttcaacgacaccctgaacgtgtacctgaccgcccacaatgcactgggctctagcctgcacaccgcaggagtggatctggcaaagtccctgagaatcgccgccaagatctactctgaggcagacgaggcatggaggaaggcaatcgatggcctgttcaccaggggcagaaagcggcgcagcctgaggctggtggtgggcgccctgctgctggtgctggcctttgccggcggctatgcagtggcagcctgtaagaccgtgacactgaccgtggacggaaccgcaatgagggtgaccacaatgaagagcagagtgatcgatatcgtggaggagaacggcttttccgtggacgatagggacgatctgtaccctgcagcaggagtgcaggtgcacgacgccgatacaatcgtgctgaggagatctaggccactgcagatcagcctggacggccacgatgccaagcaagtgtggaccacagcatccaccgtggacgaggccctggcacagctggcaatgacagataccgcacctgcagcagcctcccgggcctctcgcgtgccactgtctggaatggcactgccagtggtgagcgccaagacagtgcagctgaatgacggcggcctggtgaggaccgtgcacctgcctgcaccaaacgtggcaggcctgctgagcgccgcaggagtgccactgctgcagtccgatcacgtggtgcctgcagcaaccgcaccaatcgtggagggaatgcagatccaggtgacacggaatcgcatcaagaaggtgaccgagcgcctgccactgccccctaatgcaaggcgcgtggaggaccctgagatgaacatgtctagggaggtggtggaggaccccggagtgcctggcacacaggatgtgaccttcgccgtggccgaagtgaatggagtggagacaggccgcctgcctgtggcaaacgtggtggtgaccccagcacacgaggcagtggtgagggtgggaacaaagccaggaaccgaggtgccacccgtgatcgacggctccatctgggatgcaatcgcaggatgcgaggccggcggaaattgggccatcaacacaggcaatggctactatggcggcgtgcagtttgaccagggaacctgggaggcaaacggcggcctgagatacgcccctcgggccgatctggcaacacgcgaggagcagatcgcagtggcagaggtgaccaggctgagacagggatggggcgcctggcccgtgtgcgcagccagagccggcgccaggagaggccggaagcggcgctctgtgagcacatatcgcagccccgacagggcatggcaggccctggcagatggaaccaggagagccatcgtggagagactggcacacggacctctggcagtgggagagctggccagagatctgcccgtgtcccggcctgccgtgtctcagcacctgaaggtgctgaagacagcaaggctggtgtgcgacaggccagcaggaaccaggcgcgtgtaccagctggaccccacaggcctggccgccctgcgcaccgacctggatagattctggacacgggccctgaccggatatgcacagctgatcgactccgagggcgacgatacacggggccgcaagaggagatcctctacccagaggccaagacactctggcatcagggcagtgggaccatacgcatgggcaggcaggtgtggaaggatcggccggtggggcgtgcaccaggaggcaatgatgaacctggccatctggcaccctcgcaaggtgcagtccgccacaatctatcaggtgaccgacaggagccacgatggaaggacagccagggtgccaggcgacgagatcacaagcaccgtgtccggatggctgtccgagctgggaacccagtctcctctggcagatgagctggccagagccgtgcggatcggcgattggcccgccgcctacgctattggggaacacctgtccgtggagattgctgtcgcagtgtgataa

SEQ ID NO: 61 - protein, 1451 aa, synthetic construct
MDWTWILFLVAAATRVHSTPVRPPHTPDPLNLRGPLDGPRWRRAEPAQSRRPGRSRPGGAPLRYHRTGVGMSRTGHGSRPVPPATTVGLALLAAAITLWLGLVAQFGQMITGGSADGSADSTGRVPDRLAVVRVETGESLYDVAVRVAPNAPTRQVADRIRELNGLQTPALAVGQTLIAPVGRGRKRRSQQTAWAPRTSGIAGCGAGGVVMAIASVTLVTDTPGRVLTGVAALGLILFASATWRARPRLAITPDGLAIRGWFRTQLLRHSNIKIIRIDEFRRYGRLVRLLEIETVSGGLLILSRWDLGTDPVEVLDALTAAGYAGRGQRRGRKRRSAVNRRVPRVRDLAPLLQFNRPQFDTSKRRLGAALTIQDLRRIAKRRTPRAAFDYADGGAEDELSIARARQGFRDIEFHPTILRDVTTVCAGWNVLGQPTVLPFGIAPTGFTRLMHTEGEIAGARAAAAAGIPFSLSTLATCAIEDLVIAVPQGRKWFQLYMWRDRDRSMALVRRVAAAGFDTMLVTVDVPVAGARLRDVRNGMSIPPALTLRTVLDAMGHPRWWFDLLTTEPLAFASLDRWPGTVGEYLNTVFDPSLTFDDLAWIKSQWPGKLVVKGIQTLDDARAVVDRGVDGIVLSNHGGRQLDRAPVPFHLLPHVARELGKHTEILVDTGIMSGADIVAAIALGARCTLIGRAYLYGLMAGGEAGVNRAIEILQTGVIRTMRLLGVTCLEELSPRHVTQLRRLGPIGAPTRGRKRRSRLTHPTPCPENGETMIDRRRSAWRFSVPLVCLLAGLLLAATHGVSGGTEIRRSDAPRLVDLVRRAQASVNRLATEREALTTRIDSVHGRSVDTALAAMQRRSAKLAGVAAMNPVHGPGLVVTLQDAQRDANGRFPRDASPDDLVVHQQDIEAVLNALWNAGAEAIQMQDQRIIAMSIARCVGNTLLLNGRTYSPPYTIAAIGDAAAMQAALAAAPLVTLYKQYVVRFGLGYCEEVHPDLQIVGYADPVRMHFAQPAGPLDYRGRKRRSVAESSLNPSLVSRISAFLRPDWTRTVRARRFAAAGLVMLAGVAALRSNPEDDRSEVVVAAHDLRPGTALTPGDVRLEKRSATTLPDGSQADLDAVVGSTLASPTRRGEVLTDVRLLGSRLAESTAGPDARIVPLHLADSALVDLVRVGDVVDVLAAPVTDSPAALRLLATDAIVVLVSAQQKAQAADSDRVVLVALPARLANTVAGAALGQTVTLTLHRGRKRRSAVGPLRVSAGVIRLRPVRMRDGVHWSRIRLADRAHLEPWEPSADGEWTVRHTVAAWPAVCSGLRSEARNGRMLPYVIELDGQFCGQLTIGNVTHGALRSAWIGYWVPSAATGGGVATGALALGLDHCFGPVMLHRVEATVRPENAASRAVLAKVGFREEGLLRRYLEVDRAWRDHLLMAITVEEVYGSVASTLVRAGHASWP

SEQ ID NO: 62 - DNA, 4359 bp, synthetic construct
  source 1..4359: mol_type = other DNA
atggactggacttggattctgtttctggtcgccgccgctactagggtgcattcaactcctgtcaggcccccacatactcccgatcccctgaatctgagaggcccactggacggcccccggtggaggagagcagagcctgcacagtcccggcgcccaggcagatctcggcctggcggcgccccactgcgctaccacaggacaggagtgggaatgagcaggaccggacacggctccaggcctgtgccccctgccaccacagtgggcctggccctgctggcagcagccatcaccctgtggctgggcctggtggcacagttcggacagatgatcaccggcggctccgccgacggctccgccgattctacaggaagggtgccagacaggctggcagtggtgcgggtcgagacaggagagtctctgtatgatgtggccgtgcgcgtggcaccaaacgcaccaacaaggcaggtggcagaccgcatcagggagctgaatggcctgcagacccctgccctggcagtgggacagacactgatcgcaccagtgggaagaggaaggaagaggagatctcagcagacagcctgggccccacggaccagcggaatcgcaggatgcggagccggcggcgtggtcatggccatcgccagcgtgaccctggtgaccgatacaccaggacgcgtgctgacaggagtggccgccctgggcctgatcctgtttgcatccgccacctggagggccaggcccagactggcaatcacacctgacggcctggcaatcaggggatggttcaggacccagctgctgagacactccaatatcaagatcatccggatcgatgagtttcggcgctacggcagactggtgcggctgctggagatcgagacagtgtctggcggcctgctgatcctgagcagatgggacctgggaaccgatcccgtggaggtgctggacgcactgacagcagcaggatatgcaggaagaggacagaggagaggaaggaagcggcgctctgccgtgaacaggagagtgccccgggtgcgcgacctggcccctctgctgcagttcaataggccacagtttgataccagcaagcggcgcctgggagccgccctgacaatccaggacctgaggagaatcgcaaagaggaggacccctagagccgccttcgactacgcagatggcggagccgaggatgagctgtccatcgccagggccagacagggcttcagagacatcgagtttcaccctacaatcctgcgggatgtgaccacagtgtgcgcaggatggaacgtgctgggacagccaaccgtgctgcctttcggcatcgcaccaacaggctttaccagactgatgcacacagagggcgagatcgcaggagccagggccgcagcagcagcaggcatccccttttctctgagcacactggccacctgtgccatcgaggacctggtcatcgccgtgcctcagggcaggaagtggttccagctgtacatgtggcgggaccgcgataggagcatggcactggtgaggagagtggcagcagcaggctttgacacaatgctggtgaccgtggatgtgccagtggcaggagcaagactgagggatgtgcggaacggcatgtccatcccacccgccctgacactgaggaccgtgctggacgcaatgggacacccaaggtggtggttcgatctgctgaccacagagcccctggcctttgcctctctggacaggtggcctggaacagtgggagagtatctgaataccgtgttcgatcccagcctgacatttgacgatctggcctggatcaagtcccagtggcctggcaagctggtggtgaagggcatccagaccctggacgatgccagagccgtggtggaccggggcgtggatggaatcgtgctgtctaaccacggcggcagacagctggacagggcaccagtgcctttccacctgctgccacacgtggcccgggagctgggcaagcacacagagatcctggtggacaccggcatcatgagcggcgccgatatcgtggcagcaatcgccctgggagcaaggtgcaccctgatcggcagggcctacctgtatggcctgatggccggcggcgaggcaggagtgaacagggccatcgagatcctgcagacaggcgtgatccgcaccatgaggctgctgggcgtgacctgtctggaggagctgtctccccgccacgtgacacagctgcggcgcctgggaccaatcggagcacccacccgcggcaggaagaggagaagcaggctgacacacccaaccccctgccctgagaatggcgagacaatgatcgaccggcgcaggtccgcctggagattctctgtgcccctggtgtgcctgctggcaggcctgctgctggcagcaacccacggcgtgagcggcggcacagagatcagacggtccgacgcccctagactggtggatctggtgcgcagggcacaggcaagcgtgaacaggctggcaaccgagagggaggccctgaccacacgcatcgacagcgtgcacggcaggtccgtggatacagccctggcagcaatgcagagacggtccgccaagctggcaggagtggcagcaatgaaccctgtgcacggaccaggcctggtggtgaccctgcaggacgcacagagagatgccaatggcagatttccccgggacgcatcccctgacgatctggtggtgcaccagcaggatatcgaggccgtgctgaacgcactgtggaatgcaggagcagaggcaatccagatgcaggaccagcgcatcatcgccatgtccatcgccagatgcgtgggcaacaccctgctgctgaatggccggacatactctcctccatataccatcgccgccatcggcgatgcagcagcaatgcaggccgccctggcagcagcccctctggtgaccctgtacaagcagtatgtggtgcggttcggcctgggctactgtgaggaggtgcacccagacctgcagatcgtgggctatgccgatcccgtgcgcatgcactttgcacagcctgcaggaccactggactacagaggaaggaagcgcaggagcgtggcagagagctccctgaacccaagcctggtgtccagaatctctgccttcctgcggcctgattggacccgcacagtgagggcaagacggtttgcagcagcaggcctggtcatgctggcaggagtggccgccctgagatccaatccagaggacgatcggtctgaggtggtggtggcagcacacgacctgaggccaggaacagccctgaccccaggcgatgtgcgcctggagaagaggtctgccaccacactgcctgacggcagccaggcagacctggatgcagtggtgggcagcacactggcatccccaaccaggaggggcgaggtgctgaccgacgtgagactgctgggctctcggctggcagagagcacagcaggacctgatgcaagaatcgtgccactgcacctggcagacagcgccctggtggatctggtgcgggtgggcgacgtggtggatgtgctggcagcaccagtgaccgactccccagccgccctgagactgctggccacagatgccatcgtggtgctggtgtccgcccagcagaaggcacaggcagccgactctgatagagtggtgctggtggccctgcccgcccggctggccaataccgtggcaggagccgccctgggacagaccgtgacactgaccctgcacaggggaaggaagagacggtccgccgtgggaccactgagggtgtctgccggcgtgatcagactgcggcccgtgcgcatgagggacggagtgcactggtctagaatccggctggcagatagagcacacctggagccatgggagcctagcgccgacggagagtggacagtgcgccacaccgtggcagcatggccagccgtgtgctctggcctgaggagcgaggcaagaaacggaaggatgctgccctacgtgatcgagctggatggccagttctgtggccagctgacaatcggcaatgtgacccacggcgccctgaggagcgcctggatcggctattgggtgccttccgccgcaaccggcggcggcgtggcaaccggcgccctggccctgggcctggaccactgtttcggacctgtgatgctgcacagggtggaggcaaccgtgaggccagagaacgcagccagccgcgccgtgctggccaaagtgggctttagggaggagggcctgctgcgcaggtatctggaggtggaccgcgcctggagggatcacctgctgatggccatcaccgtggaggaggtctacgggagcgtcgcaagcacactggtcagagcaggacacgcaagctggccttgataa

SEQ ID NO: 63 - protein, 1176 aa, synthetic construct
MDWTWILFLVAAATRVHSTDVSRKIRAWGRRLMIGTAAAVVLPGLVGLAGGAATAGAFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAGRGRKRRSTEQQWNFAGIEAAASAIQGNVTSIHSLLDEGKQSLTKLAAAWGGSGSEAYQGVQQKWDATATELNNALQNLARTISEAGQAMASTEGNVTGMFARGRKRRSIATTRDREGATMITFRLRLPCRTILRVFSRNPLVRGTDRLEAVVMLLAVTVSLLTIPFAAAAGTAVQDSRSHVYAHQAQTRHPATATVIDHEGVIDSNTTATSAPPRTKITVPARWVVNGIERSGEVNAKPGTKSGDRVGIWVDSAGQLVDEPAPPARAIADAALAALGLWLSVAAVAGALLALTRAILIRVRNASWQHDIDSLFCTQRRGRKRRSTTARDIMNAGVTCVGEHETLTAAAQYMREHDIGALPICGDDDRLHGMLTDRDIVIKGLAAGLDPNTATAGELARDSIYYVDANASIQEMLNVMEEHQVRRVPVISEHRLVGIVTEADIARHLPEHAIVQFVKAICSPMALASRGRKRRSLRLVVGALLLVLAFAGGYAVAACKTVTLTVDGTAMRVTTMKSRVIDIVEENGFSVDDRDDLYPAAGVQVHDADTIVLRRSRPLQISLDGHDAKQVWTTASTVDEALAQLAMTDTAPAAASRASRVPLSGMALPVVSAKTVQLNDGGLVRTVHLPAPNVAGLLSAAGVPLLQSDHVVPAATAPIVEGMQIQVTRNRIKKVTERLPLPPNARRVEDPEMNMSREVVEDPGVPGTQDVTFAVAEVNGVETGRLPVANVVVTPAHEAVVRVGTKPGTEVPPVIDGSIWDAIAGCEAGGNWAINTGNGYYGGVQFDQGTWEANGGLRYAPRADLATREEQIAVAEVTRLRQGWGAWPVCAARAGAR

SEQ ID NO: 64 - DNA, 3534 bp, synthetic construct
  source 1..3534: mol_type = other DNA
atggactggacttggattctgtttctggtcgccgccgctacacgggtgcactcaaccgatgtcagcaggaagattagggcttggggcaggagactgatgatcggcacagccgccgccgtggtgctgccaggcctggtgggcctggccggcggagccgcaaccgcaggcgccttcagcaggccaggcctgccagtggagtacctgcaggtgccttccccatctatgggcagagatatcaaggtgcagtttcagtctggcggcaacaatagccctgccgtgtacctgctggatggcctgagggcccaggacgattataatggctgggacatcaacaccccagccttcgagtggtactatcagagcggcctgtccatcgtgatgccagtgggcggccagagctccttttacagcgactggtattccccagcatgcggcaaggcaggatgtcagacatataagtgggagacattcctgacctctgagctgccacagtggctgagcgccaatagagccgtgaagccaaccggctccgccgcaatcggcctgtctatggcaggctctagcgccatgatcctggcagcctaccaccctcagcagtttatctatgcaggcagcctgtccgccctgctggaccccagccagggaatgggaccttccctgatcggcctggcaatgggcgacgccggcggatacaaggcagcagatatgtggggaccctcctctgaccctgcatgggagcggaacgatccaacccagcagatccccaagctggtggccaacaatacacgcctgtgggtgtattgcggaaacggaaccccaaatgagctgggcggcgccaatatccctgccgagttcctggagaattttgtgcggagctccaacctgaagttccaggatgcatacaacgcagccggcggacacaacgccgtgttcaattttccccctaacggcacacacagctgggagtattggggcgcccagctgaacgcaatgaagggcgacctgcagtctagcctgggagcaggaaggggaaggaagcggcgcagcaccgagcagcagtggaattttgcaggaatcgaggcagcagcatccgccatccagggcaacgtgacatctatccacagcctgctggatgagggcaagcagtccctgaccaagctggcagcagcatggggcggctctggcagcgaggcataccagggagtgcagcagaagtgggacgcaaccgccacagagctgaacaatgccctgcagaatctggcaaggaccatcagcgaggcaggacaggccatggcctccacagagggcaacgtgaccggcatgttcgccaggggcagaaagaggagaagcatcgcaaccacacgggaccgcgagggagcaacaatgatcaccttcaggctgagactgccctgtcggacaatcctgcgcgtgttttctaggaatcctctggtgagaggcaccgataggctggaggcagtggtcatgctgctggcagtgacagtgagcctgctgaccatcccctttgccgcagcagcaggaacagcagtgcaggacagcaggagccacgtgtacgcacaccaggcacagaccaggcaccctgcaaccgcaacagtgatcgatcacgagggcgtgatcgactctaacaccacagccacaagcgccccaccccgcacaaagatcaccgtgcccgccagatgggtggtgaatggcatcgagagatccggcgaggtgaacgccaagcctggaaccaagtctggcgaccgggtgggaatctgggtggatagcgccggacagctggtggacgagcccgcccctccagccagggccatcgcagacgccgccctggccgccctgggcctgtggctgtccgtggccgccgtggccggcgccctgctggccctgacccgggccatcctgatccgggtgcgcaatgcctcctggcagcacgatatcgactctctgttctgcacacagaggaggggaaggaagaggagatctaccacagcccgcgatatcatgaacgcaggagtgacctgcgtgggagagcacgagacactgacagcagcagcacagtacatgagggagcacgacatcggcgccctgccaatctgcggcgacgatgacaggctgcacggcatgctgacagatagagacatcgtgatcaagggcctggcagcaggcctggaccccaataccgcaacagcaggagagctggcccgcgattccatctactatgtggacgccaatgcctctatccaggagatgctgaacgtgatggaggagcaccaggtgcggagggtgcccgtgatcagcgagcacaggctggtgggcatcgtgaccgaggccgacatcgccagacacctgcctgagcacgccatcgtgcagttcgtgaaggccatctgctccccaatggccctggcctctaggggcaggaaaaggagatccctgaggctggtggtgggcgccctgctgctggtgctggcctttgccggcggctacgcagtggcagcctgtaagaccgtgacactgaccgtggatggcaccgccatgagagtgaccacaatgaagagcagagtgatcgacatcgtggaggagaacggcttctctgtggatgacagggatgacctgtatccagcagcaggagtgcaggtgcacgatgcagacacaatcgtgctgcggcgcagcagacccctgcagatctccctggatggccacgacgccaagcaagtgtggaccacagcaagcaccgtggatgaggccctggcacagctggcaatgacagacaccgcacctgcagcagcctccagggcctctagagtgccactgtctggaatggcactgccagtggtgagcgccaagacagtgcagctgaatgatggcggcctggtgagaaccgtgcacctgccagcccctaacgtggcaggcctgctgagcgccgcaggagtgccactgctgcagtccgaccacgtggtgcctgcagcaaccgcaccaatcgtggagggaatgcagatccaggtgacacggaaccgcatcaagaaggtgaccgagaggctgcctctgccccctaatgccaggagagtggaggacccagagatgaacatgtctagagaggtggtggaggaccccggagtgccaggaacccaggacgtgaccttcgccgtggccgaagtgaatggagtggagacaggccggctgcctgtggcaaacgtggtggtgaccccagcacacgaggcagtggtgcgcgtgggaacaaagccaggaaccgaggtgccacccgtgatcgatggctccatctgggacgcaatcgcaggatgtgaggccggcggaaattgggccatcaacacaggcaatggctactatggcggcgtgcagtttgatcagggaacctgggaggcaaacggcggcctgcgctacgcccctagggccgacctggcaaccagggaggagcagatcgcagtggcagaggtgacaaggctgagacaggggtggggagcatggcccgtgtgcgctgcaagagctggagcaagatgataa

SEQ ID NO: 65 - protein, 771 aa, synthetic construct
MDWTWILFLVAAATRVHSVSQSMYSYPAMTANVGDMAGYTGTTQSLGADIASERTAPSRACQGDLGMSHQDWQAQWNQAMEALARAYRRCRRALRQIGVLERPVGDSSDCGTIRVGSFRGRWLDPRHAGPATAADAGDRGRKRRSVADTIQVTPQMLRSTANDIQANMEQAMGIAKGYLANQENVMNPATWSGTGVVASHMTATEITNELNKVLTGGTRLAEGLVQAAALMEGHEADSQTAFQALFGASHGSRGRKRRSTINYQFGDVDAHGAMIRAQAGLLEAEHQAIVRDVLAAGDFWGGAGSVACQEFITQLGRNFQVIYEQANAHGQKVQAAGNNMAQTDSAVGSSWARGRKRRSVSTPNTLNADFDLMRSVAGITDARNEEIRAMLQAFIGRMSGVPPSVWGGLAAARFQDVVDRWNAESTRLYHVLHAIADTIRHNEAALREAGQIHARHIAAAGGDLRGRKRRSTINYQFGDVDAHGAMIRAQAGSLEAEHQAIISDVLTASDFWGGAGSAACQGFITQLGRNFQVIYEQANAHGQKVQAAGNNMAQTDSAVGSSWARGRKRRSTEQQWNFAGIEAAASAIQGNVTSIHSLLDEGKQSLTKLAAAWGGSGSEAYQGVQQKWDATATELNNALQNLARTISEAGQAMASTEGNVTGMFARGRKRRSAEMKTDAATLAQEAGNFERISGDLKTQIDQVESTAGSLQGQWRGAAGTAAQAAVVRFQEAANKQKQELDEISTNIRQAGVQYSRADEEQQQALSSQMGF

SEQ ID NO: 66 - DNA, 2319 bp, synthetic construct
  source 1..2319: mol_type = other DNA
atggactggacctggattctgttcctggtggccgccgccacaagagtgcactctgtgagccagtccatgtacagctatcctgccatgaccgccaatgtgggcgacatggccggctacacaggcaccacacagtccctgggagcagatatcgcatctgagaggaccgcaccaagccgcgcctgccagggcgacctgggaatgtcccaccaggattggcaggcccagtggaaccaggcaatggaggccctggccagggcctataggagatgcaggcgcgccctgaggcagatcggcgtgctggagcgcccagtgggcgacagctccgattgtggcaccatccgcgtgggcagctttaggggcagatggctggacccaaggcacgcaggaccagcaacagcagcagacgcaggcgataggggaaggaagaggagaagcgtggcagacaccatccaggtgacaccccagatgctgcgctccaccgccaatgatatccaggccaacatggagcaggccatgggcatcgccaagggctacctggccaaccaggagaatgtgatgaaccctgcaacctggagcggaacaggagtggtggcatcccacatgaccgccacagagatcacaaatgagctgaacaaggtgctgaccggcggcacaaggctggcagagggactggtgcaggcagccgccctgatggagggccacgaggccgattctcagaccgcattccaggccctgtttggagcaagccacggctccaggggcagaaagcggcgcagcacaatcaactaccagttcggcgacgtggatgcacacggagccatgatcagggcacaggcaggactgctggaggcagagcaccaggcaatcgtgagggacgtgctggcagcaggcgatttttggggaggagcaggctccgtggcatgccaggagttcatcacccagctgggccgcaattttcaggtcatctacgagcaggccaacgcacacggacagaaggtgcaggcagcaggcaacaatatggcccagacagactctgccgtgggctctagctgggccaggggcaggaagaggagatctgtgagcacccccaatacactgaacgccgacttcgatctgatgagaagcgtggccggcatcaccgatgccaggaatgaggagatcagagccatgctgcaggccttcatcggaaggatgagcggagtgccaccttccgtgtggggaggactggccgccgccaggtttcaggacgtggtggatagatggaatgccgagtccacccggctgtaccacgtgctgcacgccatcgccgacacaatccgccacaacgaggccgccctgagggaggccggccagatccacgcaaggcacatcgcagcagcaggaggcgatctgagaggaagaaagcggcgcagcaccatcaactatcagtttggcgacgtggacgcacacggagcaatgattagagcacaggcaggctccctggaggccgaacaccaggccatcatctccgacgtgctgaccgcctctgacttctggggaggagcaggcagcgccgcatgtcagggcttcatcacacagctgggcaggaacttccaggtcatctacgaacaggctaatgcccatggccagaaagtccaggccgcaggcaacaatatggcacagaccgactctgccgtgggctcctcttgggccaggggcagaaaaaggagaagcacagagcagcagtggaatttcgcaggaatcgaggcagcagcatccgccatccagggcaacgtgacctctatccacagcctgctggacgagggcaagcagtctctgacaaagctggcagcagcatggggaggctccggctctgaggcatatcagggcgtgcagcagaagtgggatgccaccgccacagagctgaacaatgccctgcagaatctggccagaaccatctctgaggcaggacaggcaatggcaagcaccgagggcaacgtgacaggcatgttcgccaggggcaggaaacggcgctccgccgagatgaagaccgacgcagccacactggcacaggaggcaggcaactttgagcggatctctggcgacctgaagacccagatcgatcaggtggagtccacagccggctctctgcagggccagtggagaggagcagcaggaaccgcagcacaggcagcagtggtgcggttccaggaggccgccaataagcagaagcaggagctggacgagatctccacaaacatcagacaggccggcgtgcagtattctcgggccgatgaggagcagcagcaggccctgagctcccagatgggcttttgatga

SEQ ID NO: 67 - protein, 113 aa, synthetic construct
MDWTWILFLVAAATRVHSSQIMYNYPAMMAHAGDMAGYAGTLQSLGADIASEQAVLSSAWQGDTGITYQGWQTQWNQALEDLVRAYQSMSGTHESNTMAMLARDGAEAAKWGG

SEQ ID NO: 68 - DNA, 345 bp, synthetic construct
  source 1..345: mol_type = other DNA
atggactggacctggattctgttcctggtggcagcagcaacaagggtgcacagctcccagatcatgtacaactatcccgccatgatggcacacgcaggcgacatggcaggatacgcaggcaccctgcagtccctgggagcagatatcgcctctgagcaggccgtgctgtctagcgcctggcagggcgacaccggcatcacataccagggctggcagacacagtggaaccaggccctggaggatctggtgagggcctatcagtctatgagcggcacccacgagtctaatacaatggccatgctggccagagacggagcagaggcagcaaagtggggaggatgatga

SEQ ID NO: 69 - protein, 18 aa, synthetic construct
MDWTWILFLVAAATRVHS